SEQUENCE LISTING (2 sequences)

SEQ ID NO: 1 - protein, 417 aa, synthetic construct
  REGION 1..417: note = amino acid fragment of 62-478 of human vitronectin
VTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRAMWLSLFSSEESNLGANNYDDYRMDWLVPATCEPIQSVFFFSGDKYYRVNLRTRRVDTVDPPYPRSIAQYWLGCPAPGHL

SEQ ID NO: 2 - protein, 379 aa, synthetic construct
  REGION 1..379: note = amino acid fragment of 20 to 398 of human vitronectin
DQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSR

(19) United States
(12) Patent Application Publication
(10) Pub. No.: US 2024/0417691 A1
(43) Pub. Date: Dec. 19, 2024

(54) METHOD FOR SUSPENSION CULTURE OF ADHERENT CELLS WITH STIRRING

(71) Applicant: NISSAN CHEMICAL CORPORATION, Tokyo (JP)

(72) Inventors: Masashi IWAKAMI, Shiraoka (JP); Daisuke HATANAKA, Funabashi (JP); Katsuhiko KIDA, Shiraoka (JP)

(73) Assignee: NISSAN CHEMICAL CORPORATION, Tokyo (JP)

(21) Appl. No.: 18/701,379

(22) PCT Filed: Oct. 14, 2022
(86) PCT No.: PCT/JP2022/038382

§ 371 Date: Apr. 15, 2024

(30) Foreign Application Priority Data
Oct. 15, 2021 (JP) 2021-169860
Feb. 18, 2022 (JP) 2022-023397

(51) Int. Cl. C12N 5/0775; A61K 35/28; A61P 29/00; C12N 5/00

(52) U.S. Cl. CPC C12N 5/0665; A61K 35/28; A61P 29/00; C12N 5/0006; C12N 2527/00; C12N 2533/72; C12N 2533/78; C12N 2533/90

(57) ABSTRACT

The present invention provides a method for culturing an adherent cell, including a step of suspension culturing the adherent cell in a medium containing a nanofiber composed of a water-insoluble polysaccharide, wherein the culture is performed along with stirring.

### TECHNICAL FIELD

The present invention relates to a method for suspension culturing adherent cells along with stirring and the like.

### BACKGROUND ART

In recent years, methods for transplanting or injecting cells into living bodies have been developed mainly in the fields of medicine and beauty. Among the cells, somatic stem cells and progenitor cells are attracting attention because they have a lower tumorigenicity risk, a shorter differentiation period, and the like compared to pluripotent stem cells.

When these cells are utilized, it is necessary to provide them in good condition and in a large number. As a method therefor, a method including culturing and proliferating stem cells and the like in a state of being adhered to a microcarrier and the like is known.

However, currently available microcarriers settle in the culture medium under static conditions and need to be stirred during culture. Due to the stirring, a problem has been pointed out that cell death occurs by collision between microcarriers and the like. In addition, the efficiency of cell proliferation is not sufficient, and further improvement is expected.

The present inventors have developed a medium composition for culturing animal and plant cells and/or tissues in a suspended state by using nanofibers of polysaccharides and the like having enhanced dispersibility in water (Patent Literature 1).

Furthermore, the present inventors have found that nanofibers composed of water-insoluble polysaccharides can be used as common carriers in various operations of adherent cells such as i) suspension culture, ii) differentiation induction, iii) transportation and preservation under non-frozen conditions, iv) transplantation, v) recovery of bioactive substance from culture supernatant and the like (Patent Literature 2, Patent Literature 3).

### CITATION LIST

### Patent Literature

Patent Literature 1: WO 2015/111686
Patent Literature 2: WO 2017/175751
Patent Literature 3: WO 2018/182016

### SUMMARY OF INVENTION

### Technical Problem

The present invention aims to provide a technique leading to a large-scale production of adherent cells such as somatic stem cells, progenitor cells, and the like. In addition, the present invention aims to provide a technique for efficiently producing adherent cells with good quality.

### Solution to Problem

The present inventors have conducted intensive studies in an attempt to solve the above-mentioned problems and found that suspension culture of adherent cells under conditions including stirring in a medium containing chitin nanofibers carrying vitronectin and chitosan nanofibers not only enhances proliferation of the adherent cells but also affords the adherent cells with good quality.

The present inventors have also found that adherent cells cultured under such conditions form spheres with a uniform size and show enhanced undifferentiated state and enhanced migration capability.

In addition, the present inventors have also found that spheres formed under such conditions can be easily collected with a cell strainer and can be dispersed to single cells highly efficiently using a cell dispersing agent.

In addition, the present inventors have found that mesenchymal stem cells cultured by the method of the present invention show promoted expression of specific genes and also, enhanced producing capability of extracellular vesicles.

In addition, the present inventors have studied the mechanism by which extracellular vesicle producing capability is enhanced in mesenchymal stem cells cultured by the method of the present invention.

In addition, the present inventors have found that proliferation of adherent cells can be enhanced even under conditions combining chitin nanofibers carrying vitronectin and stirring (i.e., conditions without using chitosan nanofibers), and have also found an efficient passage method under such conditions.

In addition, the present inventors have also confirmed that adherent cells do not proliferate sufficiently by stirring culture without using nanofibers and stirring culture using chitosan nanofibers alone, and that the method of the present invention can also be practiced in a large scale.

In addition, the present inventors have confirmed the physical structures of nanofibers and adherent cells in the spheres formed by the method of the present invention.

In addition, the present inventors have also found that the mesenchymal stem cells prepared by the method of the present invention have very high anti-inflammatory effects and also, high treatment effects on arthropathy.

Based on these findings, the present inventors have conducted further studies and completed the present invention.

Accordingly, the present invention provides the following.

[1] A method for culturing an adherent cell, comprising a step of suspension culturing the adherent cell in a medium comprising a nanofiber composed of a water-insoluble polysaccharide, wherein the culture is performed along with stirring.

[2] The method of [1], wherein the aforementioned stirring is performed under conditions where the nanofiber and the cell are suspended in the medium and the nanofiber and the cell are continuously moved in the system by an external force.

[3] The method of [1] or [2], wherein the aforementioned stirring is performed by a means accompanying a blade, and a rotation speed thereof is a tip speed of 0.01 to 50.0 m/min.

[4] The method of any of [1] to [3], wherein the aforementioned stirring is performed constantly during the cell culture.

[5] The method of any of [1] to [4], wherein a content of the nanofiber composed of water-insoluble polysaccharides and added to the medium is 0.0001-0.2% (w/v).

[6] The method of any of [1] to [5], wherein the nanofiber composed of water-insoluble polysaccharides carries an extracellular matrix.

[7] The method of any of [1] to [6], wherein the water-insoluble polysaccharide is at least one selected from the group consisting of chitin, cellulose, and hemicellulose.

[8] The method of [6] or [7], wherein the extracellular matrix is at least one selected from the group consisting of collagen, fibronectin, vitronectin, laminin, RGD sequence, and cadherin.

[9] The method of any of [1] to [8], wherein the adherent cell is selected from the group consisting of a stem cell, a progenitor cell, a somatic non-stem cell, a primary cultured cell, a cell line, and a cancer cell.

[10] The method of any of [1] to [9], wherein the medium further comprises a chitosan nanofiber.

[11] A method for producing a sphere of adherent cells with a uniform sphere size, comprising a step of suspension culturing adherent cells in a medium comprising a nanofiber composed of water-insoluble polysaccharides, wherein the culture is performed along with stirring.

[12] The method of [11], wherein the aforementioned stirring is performed under conditions where the nanofibers and the cells are suspended in the medium and the nanofibers and the cells are continuously moved in the system by an external force.

[13] The method of [11] or [12], wherein the aforementioned stirring is performed by a means accompanying a blade, and a rotation speed thereof is a tip speed of 0.01 to 50.0 m/min.

[14] The method of any of [11] to [13], wherein the aforementioned stirring is performed constantly during the cell culture.

[15] The method of any of [11] to [14], wherein a content of the nanofiber composed of water-insoluble polysaccharides and added to the medium is 0.0001-0.2% (w/v).

[16] A method for isolating spheres, comprising a step of subjecting a suspension of spheres produced by the method of any of [11] to [15] to a cell strainer.

[17] A method for dispersing adherent cells in the form of spheres into single cells, comprising
a first step of suspension culturing adherent cells in a medium comprising nanofibers composed of water-insoluble polysaccharides, and
a second step of treating the spheres of the adherent cells obtained in the first step with a cell dispersing agent.

[18] A mesenchymal stem cell in which expression of at least one gene selected from the group consisting of CD55, HMOX1, TSPAN7, RAB27B, IL33, GPX3, and MFAP4 is promoted as compared with that in a mesenchymal stem cell cultured by adhesion culture.

[19] The mesenchymal stem cell of [18], wherein production of an extracellular vesicle is enhanced as compared with that in a mesenchymal stem cell cultured by adhesion culture.

[20] The mesenchymal stem cell of [19], wherein the extracellular vesicle is an exosome.

[21] A method for enhancing production of an extracellular vesicle of a mesenchymal stem cell, comprising a step of suspension culturing the mesenchymal stem cell in a medium comprising a nanofiber composed of water-insoluble polysaccharides, wherein the culture is performed along with stirring.

[22] A method for producing a mesenchymal stem cell in which production of an extracellular vesicle is enhanced, comprising a step of suspension culturing the mesenchymal stem cell in a medium comprising a nanofiber composed of water-insoluble polysaccharides, wherein the culture is performed along with stirring.

[23] The method of [21] or [22], wherein the extracellular vesicle is an exosome.

[24] A therapeutic agent for an inflammatory disease, comprising the mesenchymal stem cell of any of [18] to [20].

[25] A method for treating an inflammatory disease in a subject, comprising administering the mesenchymal stem cell of any of [18] to [20] to the subject having the inflammatory disease.

[26] Use of the mesenchymal stem cell of any of [18] to [20] in the manufacture of a medicament for treating an inflammatory disease.

[27] The mesenchymal stem cell of any of [18] to [20] for use in treating an inflammatory disease.

### Advantageous Effects of Invention

According to the present invention, adherent cells can be produced efficiently.

According to the present invention, moreover, adherent cell spheres with a uniform size can be produced. According to the present invention, moreover, an adherent cell with promoted undifferentiated state and promoted migration capability can be produced. According to the present invention, moreover, adherent cell spheres with a uniform size can be isolated. Furthermore, the spheres obtained by the present invention can be dispersed into single cells extremely efficiently. According to the present invention, moreover, a mesenchymal stem cell favorable for regenerative medicine and having enhanced extracellular vesicle producing capability can be produced.

### BRIEF DESCRIPTION OF DRAWINGS

FIG. 1 shows photographs observing the spheres obtained by suspension culturing human umbilical cord-derived mesenchymal stem cells in a medium containing chitin nanofibers carrying vitronectin and chitosan nanofibers under respective conditions (conditions 4 and 5) of Experimental Example 3.

FIG. 2 shows diagrams presenting the results of image analysis (sphere extraction image) of the fluorescent stained images of FIG. 1.

FIG. 3 shows diagrams presenting the distribution of sphere sizes prepared under respective conditions (conditions 4 and 5) of Experimental Example 3.

FIG. 4 shows photographs observing the spheres obtained by suspension culturing human umbilical cord-derived mesenchymal stem cells in a medium containing chitin nanofibers carrying vitronectin and chitosan nanofibers under respective conditions of Experimental Example 4.

FIG. 5 shows photographs taken using Cell3iMagerduos (manufactured by SCREEN Holdings Co., Ltd.) of cells suspension cultured under respective conditions of Experimental Example 4, and thereafter seeded in well plates.

FIG. 6 shows diagrams presenting sphere extraction images obtained as a result of image analysis in Experimental Example 4.

FIG. 7 shows diagrams presenting the number of spheres and the average diameter of the spheres under respective conditions of Experimental Example 4.

FIG. 8 shows photographs presenting the state of spheres under respective conditions of Experimental Example 5.

FIG. 9 shows diagrams presenting the substrate and cells in the filtrates in Experimental Example 6.

FIG. 10 shows photographs presenting the state of cells under respective conditions of Experimental Example 6.

FIG. 11 is a diagram presenting the connection style of the Rotea Single Use Kit used for dispersing spheres and collecting single cells in Experimental Example 7.

FIG. 12 shows photographs presenting the state of the spheres or single cells at respective stages of Experimental Example 7.

FIG. 13 shows photographs presenting the appearance of cells being cultured (day 0 and day 3 of culture) in Experimental Example 10.

FIG. 14 shows diagrams presenting microscopic images of cells being cultured (day 0 and day 3 of culture) in Experimental Example 10.

FIG. 15 shows diagrams presenting bright field images and fluorescent staining images of spheres or cells after respective treatments in Experimental Example 11.

FIG. 16 shows diagrams presenting images of spheres or cells used for analysis in Experimental Example 11.

FIG. 17 shows photographs of spheres observed using an inverted microscope at respective time points after enzyme treatment in Experimental Example 12.

FIG. 18 shows photographs of the state of spheres or cells observed using an inverted microscope after respective treatment in Experimental Example 13.

FIG. 19 shows a diagram confirming using Western blotting the promoted protein expression of RAB27B in mesenchymal stem cells cultured using the method of the present invention.

FIG. 20 shows a diagram confirming using Western blotting the protein expression of NFE2 L2, P65, and phosphorylated P65 (p-P65) in mesenchymal stem cells cultured using the method of the present invention.

FIG. 21 shows diagrams confirming using Western blotting the protein expression level of RAB27B when various siRNA treatments were performed in mesenchymal stem cells cultured using the method of the present invention.

FIG. 22 shows diagrams presenting the results when mesenchymal stem cells cultured using substrate 2 were passaged under condition 1 (simple addition of fresh medium containing substrate 2), condition 2 (partial single cell dispersion by applying physical shearing force to spheres, followed by addition of fresh medium containing substrate 2), or condition 3 (addition of fresh medium not containing substrate).

FIG. 23 shows diagrams presenting the results when mesenchymal stem cells cultured using substrate 1 or substrate 2 were passaged by a specific operation (operation 1 to operation 3).

FIG. 24 shows diagrams presenting the results when mesenchymal stem cells were suspension cultured under stirring conditions using various substrates (substrate 1 to substrate 3).

FIG. 25 shows diagrams presenting the shapes of spheres formed when the method of the present invention was performed on a large scale (1 L).

FIG. 26 shows diagrams presenting the images of sections of spheres prepared using substrate 1 or substrate 2.

### DESCRIPTION OF EMBODIMENTS

The present invention is described in detail in the following.

### 1. Culture Method of Adherent Cells

The present invention provides a method for culturing an adherent cell, including a step of suspension culturing the adherent cell in a medium containing a nanofiber composed of a water-insoluble polysaccharide, wherein the culture is performed along with stirring (hereinafter sometimes referred to as “the method of the present invention”, etc.).

In the method of the present invention, adherent cell is a cell that requires a scaffold such as a container wall and the like for survival and proliferation.

In the method of the present invention, the adherent cell is not particularly limited and, for example, stem cell, progenitor cell, somatic non-stem cell, primary cultured cell, cell line, cancer cell and the like can be mentioned. Stem cell is a cell concurrently having an ability to replicate itself, and an ability to differentiate into other plural lineages. Examples of the adherent stem cell include, but are not limited to, somatic stem cell and the like such as mesenchymal stem cell, neural stem cell, hematopoietic stem cell, liver stem cell, pancreas stem cell, muscle stem cell, germ stem cell, intestinal stem cell, cancer stem cell, hair follicle stem cell and the like. Mesenchymal stem cell is a stem cell having differentiation potency into all or some of osteocyte, chondrocyte and adipocyte. Mesenchymal stem cell is present in a tissue such as bone marrow, peripheral blood, cord blood, adipose tissue and the like at a low frequency and can be isolated from these tissues by a known method. Progenitor cell is a cell on the way to differentiate from the aforementioned stem cell into a particular somatic cell or reproductive cell. Examples of the adherent progenitor cell include, but are not limited to, pre-adipocyte, cardiac muscle progenitor cell, endothelial progenitor cell, neural progenitor cell, liver progenitor cell, pancreas progenitor cell, kidney progenitor cell and the like. Examples of the adherent somatic non-stem cell include, but are not limited to, fibroblast, osteocyte, bone pericyte, keratinocyte, adipocyte, mesenchymal cell, epithelial cell, epidermal cell, endothelial cell, vascular endothelial cell, hepatocyte, chondrocyte, cumulus cell, neural cell, glial cell, neuron, oligodendrocyte, microglia, astrocyte, heart cell, esophagus cell, muscle cell (e.g., smooth muscle cell or skeletal muscle cell), pancreas beta cell, melanin cell, and the like. Primary cultured cell is a cell after separation of cells and tissues from a living body and in a state of culture before performing the first passage. The primary cultured cell may be a cell collected from any tissue, for example, skin, kidney, spleen, adrenal gland, liver, lung, ovary, pancreas, uterus, stomach, colon, small intestine, large intestine, bladder, prostate, testis, thymus, muscle, bond tissue, bone, cartilage, blood vessel tissue, blood, heart, eye, brain, nerve tissue and the like. Cell lines are cells that have acquired infinite proliferative capacity by an artificial operation in vitro. The adherent cell in the method of the present invention is preferably a stem cell or progenitor cell, more preferably a mesenchymal stem cell.

The derivation of the adherent cell in the method of the present invention is not particularly limited, and the cell may be derived from any animal or plant. Examples of the animal include fish, amphibian, reptiles, birds, pancrustacea, hexapoda, mammals and the like, with preference given to mammal. Examples of the mammal include, but are not limited to, rat, mouse, rabbit, guinea pig, squirrel, hamster, vole, platypus, dolphin, whale, dog, cat, goat, bovine, horse, sheep, swine, elephant, common marmoset, squirrel monkey, Macaca mulatta, chimpanzee, human and the like. The plant is not particularly limited as long as the collected cells can be applied to liquid culture. Examples thereof include, but are not limited to, plants (e.g., ginseng, periwinkle, henbane, coptis, belladonna etc.) producing crude drugs (e.g., saponin, alkaloids, berberine, scopolin, phytosterol etc.), plants (e.g., blueberry, safflower, madder, saffron etc.) producing dye or polysaccharide (e.g., anthocyanin, safflower dye, madder dye, saffron dye, flavones etc.) to be a starting material for cosmetic or food, or plants producing a pharmaceutical active pharmaceutical ingredient, and the like.

In the present specification, nanofiber refers to a fiber having an average fiber diameter (D) of 0.001 to 1.00 μm. The average fiber diameter of the nanofiber to be used in the present invention is preferably 0.005 to 0.50 μm, more preferably 0.01 to 0.05 μm, further preferably 0.01 to 0.02 μm.

In the method of the present invention, the aspect ratio (L/D) of the nanofiber to be used is not particularly limited and is obtained from average fiber length/average fiber diameter, and is generally 2-500, preferably 5-300, more preferably 10-250.

In the present specification, the average fiber diameter (D) of the nanofiber is determined as follows. First, a hydrophilizing treatment of a collodion carry film manufactured by Okenshoji Co., Ltd. is performed for 3 min by an ion cleaner (JIC-410) manufactured by JEOL Ltd., several drops of a nanofiber dispersion (diluted with ultrapure water) to be the evaluation target is added dropwise, and dried at room temperature. This is observed under a transmission electron microscope (TEM, H-8000) (10,000-fold) manufactured by Hitachi, Ltd. at an accelerating voltage 200 kV. Using the obtained image, the fiber diameter of each one of the nanofibers (specimen number: 200-250) is measured, and the mean thereof is taken as the average fiber diameter (D).

In addition, the average fiber length (L) is determined as follows. A nanofiber dispersion to be the evaluation target is diluted to 100 ppm with pure water, and nanofibers are uniformly dispersed using an ultrasonic cleaner. The nanofiber dispersion is cast on a silicon wafer subjected in advance to a hydrophilizing treatment of the surface with conc. sulfuric acid, dried at 110° C. for 1 hr and used as a sample. Using an image obtained by observing the obtained sample under a scanning electron microscope (SEM, JSM-7400F) (2,000-fold), the fiber length of each one of the nanofibers (specimen number: 150-250) is measured, and the mean thereof is taken as the average fiber length (L).

In a preferable embodiment, the nanofiber is, upon mixing with a liquid medium, uniformly dispersed in the liquid while maintaining the primary fiber diameter, substantially retains the cells attached to the nanofiber without substantially increasing the viscosity of the liquid, and shows an effect of preventing sediment thereof.

The nanofiber to be used in the method of the present invention is constituted of water-insoluble polysaccharides. Polysaccharides mean glycopolymers wherein not less than 10 single saccharides (e.g., triose, tetrose, pentose, hexsauce, heptose etc.) are polymerized.

Examples of the water-insoluble polysaccharides include, but are not limited to, celluloses such as cellulose, hemicellulose and the like; chitinous substances such as chitin, chitosan and the like, and the like. The water-insoluble polysaccharides are preferably chitin or chitosan, more preferably chitin. In the present specification, the “nanofiber composed of chitin” is sometimes referred to as “chitin nanofiber”. The same applies to other water-insoluble polysaccharides.

The chitinous substance refers to one or more carbohydrates selected from the group consisting of chitin and chitosan. Major sugar units constituting chitin and chitosan are N-acetylglucosamine and glucosamine, respectively. Generally, chitin has a high N-acetylglucosamine content and is poorly soluble in acidic aqueous solution, and chitosan has a high glucosamine content and is soluble in acidic aqueous solution. For convenience, chitin contains not less than 50% of N-acetylglucosamine in the constituent sugar, and chitosan contains less than 50% of N-acetylglucosamine in the present specification.

As the starting material of chitin, many biological resources such as shrimps, crabs, insect, shells, mushrooms and the like can be used. The chitin to be used in the present invention may be one having α-form crystal structure such as chitin derived from crab shell, shrimp shell and the like, or one having β-form crystal structure such as chitin derived from cuttlebones and the like. The test of crabs and shrimps is often regarded as industrial waste and preferable as a starting material since it is easily available and effectively used. On the other hand, it requires a protein removing step and a decalcification step to remove protein, minerals and the like contained as impurities. In the present invention, therefore, purified chitin that underwent a matrix removal treatment is preferably used. Purified chitin is commercially available. The starting material for the chitin nanofiber to be used in the present invention may be a chitin having any of the a type and β type crystal structures, but an a type chitin is preferable.

By pulverizing the aforementioned polysaccharides, a nanofiber constituted of the polysaccharides can be obtained. While the pulverization method is not limited, a method affording a strong shear force such as a medium stirring mill, for example, a high-pressure homogenizer, a grinder (stone mill), a bead mill and the like is preferable for subdivision to the below-mentioned fiber diameter and fiber length meeting the object of the present invention.

Of these, subdivision by a high-pressure homogenizer is preferable, and, for example, subdivision (pulverization) by the wet grinding method disclosed in JP-A-2005-270891 or JP-B-5232976 is desirable. Specifically, the starting material is pulverized by spraying a dispersion of a starting material from a pair of nozzles at a high-pressure and bombarding each other, and, for example, Star Burst system (high-pressure pulverization device manufactured by Sugino Machine Limited) or NanoVater (high-pressure pulverization device manufactured by yoshida kikai co., ltd.) is used therefor.

In the subdivision (pulverization) of a starting material by the aforementioned high-pressure homogenizer, the degree of subdivision and homogenization depends on the pressure in pumping into an ultrahigh-pressure chamber in a high-pressure homogenizer, and the number (treatment number) of passage through the ultrahigh-pressure chamber, and the concentration of the starting material in the aqueous dispersion. The pumping pressure (treatment pressure) is not particularly limited and it is generally 50-250 MPa, preferably 100-200 MPa.

While the concentration of the starting material in a aqueous dispersion during the subdividing treatment is not particularly limited, it is generally 0.1 mass %-30 mass %, preferably 1 mass %-10 mass %. While the treatment number of the subdivision (pulverization) is not particularly limited, it varies depending on the concentration of the starting material in the aforementioned aqueous dispersion. When the concentration of the starting material is 0.1-1 mass %, the treatment number of 10-100 is sufficient for pulverization, but 1-10 mass % sometimes requires about 10-1000 times of treatment.

The viscosity of the aqueous dispersion during the aforementioned subdivision treatment is not particularly limited. For example, in the case of a chitin, the viscosity of the aqueous dispersion is within the range of 1-100 mPa·S, preferably 1-85 mPa·S (by tuning fork vibration type viscometer (SV-1A, A&D Company Ltd.) under 25° C. conditions). In the case of chitosan, the viscosity of the aqueous dispersion is within the range of 0.7-30 mPa·S, preferably 0.7-10 mPa·S (by tuning fork vibration type viscometer (SV-1A, A&D Company Ltd.) under 25° C. conditions).

The preparation method of a nanofiber is described in WO 2015/111686 A1 and the like.

In one embodiment, in the method of the present invention, the nanofiber composed of water-insoluble polysaccharides can carry an extracellular matrix. In the present specification, that the nanofiber carries an extracellular matrix means a state in which the nanofiber and the extracellular matrix are attached or adsorbed without a chemical covalent bond. An extracellular matrix can be carried by nanofibers by intermolecular force, electrostatic interaction, hydrogen bond, hydrophobic interaction, or the like, though it is not limited to these. In other words, the state in which nanofibers carry an extracellular matrix is a state in which the nanofibers and the extracellular matrix remain in contact with each other without a chemical covalent bond, or the nanofibers and the extracellular matrix form a complex without a chemical covalent bond.

In the method of the present invention, the extracellular matrix carried on the nanofibers is not particularly limited as long as the desired effect can be obtained, and collagen (collagen I to XIX), fibronectin, vitronectin, laminin (laminin-1 to 12), RGD sequence, cadherin and the like can be mentioned. The selection of the extracellular matrix depends on the type of cells to be proliferated, and can be appropriately selected by those of ordinary skill in the art. For example, in the case of mesenchymal stem cells, vitronectin is preferable as the extracellular matrix. When the vitronectin is human-derived vitronectin, the amino acid sequence thereof preferably consists of 20-398 (SEQ ID NO: 2) or 62-478 (SEQ ID NO: 1). When using non-human-derived vitronectin, a region corresponding to a fragment of human-derived vitronectin can be used.

In the method of the present invention, the amount of extracellular matrix carried by the nanofiber is generally 0.001-50 mg, preferably 0.01-10 mg, more preferably 0.1-10 mg, further preferably 0.3-10 mg, further more preferably, 1-10 mg, particularly preferably 2-10 mg, per 1 g of nanofibers, though not limited to these.

In the method of the present invention, the nanofibers carrying the extracellular matrix are prepared by mixing a dispersion in which the nanofibers are dispersed in an aqueous solvent and an aqueous solution of the extracellular matrix, and allowing the mixture to stand for a given period of time as necessary. Examples of the aqueous solvent for dispersing the nanofibers include, but are not limited to, water, dimethyl sulfoxide (DMSO), and the like. As the aqueous solvent, water is preferred. The aqueous solvent may contain appropriate buffering agents and salts. In order to uniformly contact the extracellular matrix with the nanofibers, it is preferable to sufficiently mix them by a pipetting operation or the like. As the time of standing, a mixture of the nanofiber dispersion and the extracellular matrix aqueous solution may be left standing generally for 30 min or more, preferably 1 hr or more, more preferably 3 hr or more, further preferably 6 hr or more, further more preferably 9 hr or more, particularly preferably 12 hr or more. While there is no particular upper limit on the standing time, for example, an upper limit of 48 hr or less (e.g., 36 hr or less, 24 hr or less, 16 hr or less, etc.) may be set. The temperature during standing is not particularly limited, and may be generally 1-30° C., preferably 1-28° C., 1-26° C., 1-25° C., 1-24° C., 1-23° C., 1-22° C., 1-21° C., 1-20° C., 1-19° C., 1-18° C., 1-17° C., 1-16° C., or 1-15° C., more preferably 2-10° C., 5-25° C., or 15-25° C., particularly preferably 2-5° C. (e.g., 4° C.) or 15-25° C. (e.g., 20° C.).

The mixing ratio of the nanofiber composed of water-insoluble polysaccharides and the extracellular matrix varies depending on the kind of these substances to be used, and is, but not limited to, for example, 100:0.1-1, preferably, 100:0.4-0.6, in terms of the solid content weight.

The amount of the extracellular matrix carried on the nanofibers composed of water-insoluble polysaccharides can be measured by, for example, Micro BCA method, enzyme immunoassay (ELISA method) and the like, but is not limited thereto.

In a preferred embodiment, the nanofibers composed of water-insoluble polysaccharides are uniformly dispersed in a liquid medium, and the adherent cells attached to the nanofibers are suspended in the liquid medium.

In the method of the present invention, the medium containing the nanofibers carrying the extracellular matrix can be appropriately selected according to the kind and the like of the adherent cells to be used. For example, when used for the purpose of culture of mammalian adherent cells, a medium generally used for culturing mammalian cells can be used as a medium. Examples of the medium for mammalian cells include Dulbecco's Modified Eagle's medium (DMEM), hamF12 medium (Ham's Nutrient Mixture F12), DMEM/F12 medium, McCoy's 5A Medium, Eagle MEM medium (Eagle's Minimum Essential medium; EMEM), αMEM medium (alpha Modified Eagle's Minimum Essential medium; αMEM), MEM medium (Minimum Essential medium), RPMI1640 medium, Iscove's Modified Dulbecco's medium (IMDM), MCDB131 medium, William medium E, IPL41 medium, Fischer's medium, StemPro34 (manufactured by Invitrogen), X-VIVO 10 (manufactured by Cambrex Corporation), X-VIVO 15 (manufactured by Cambrex Corporation), HPGM (manufactured by Cambrex Corporation), StemSpan H3000 (manufactured by STEMCELL Technologies), StemSpan SFEM (manufactured by STEMCELL Technologies), StemlineII (manufactured by Sigma Aldrich), QBSF-60 (manufactured by Quality Biological), StemPro hESCSFM (manufactured by Invitrogen), Essential6 (registered trademark) medium (manufactured by Gibco), Essential8 (registered trademark) medium (manufactured by Gibco), Essential8 (registered trademark) Flex medium (manufactured by Thermo Fisher), StemFlex medium (manufactured by Thermo Fisher), StemScale (registered trademark) PSC Suspension Medium (manufactured by Thermo Fisher), mTeSR1 or 2 or Plus medium (manufactured by STEMCELL Technologies), REPRO FF or REPRO FF2 (manufactured by REPROCELL), PSGro hESC/iPSC medium (manufactured by System Biosciences), NutriStem (registered trademark) medium (manufactured by Biological Industries), MSC NutriStem (registered trademark) XF Medium (manufactured by Biological Industries), CSTI-7 medium (manufactured by Cell Science & Technology Institute), MesenPRO RS medium (manufactured by Gibco), MF-Medium (registered trademark) mesenchymal stem cell proliferation medium (manufactured by TOYOBO Co., Ltd.), mesenchymal stem cell serum-free medium (manufactured by FUKOKU), Mesenchymal Stem Cell Growth Medium 2 (manufactured by PromoCell), Sf-900II (manufactured by Invitrogen), Opti-Pro (manufactured by Invitrogen), StemFit (registered trademark) AK02N or Basic02 or AK03N or Basic03 or Basic04 medium (manufactured by Ajinomoto Healthy Supply Co., Inc.), STEMUP medium (manufactured by Nissan Chemical Corporation), and the like.

Those of ordinary skill in the art can freely add, according to the object, sodium, potassium, calcium, magnesium, phosphorus, chlorine, various amino acids, various vitamins, antibiotic, serum, fatty acid, sugar and the like to the above-mentioned medium. For culture of mammalian cells, those of ordinary skill in the art can also add, according to the object, one or more kinds of other chemical components and biogenic substances in combination. Examples of the components to be added to a medium for mammalian cells include fetal bovine serum, human serum, horse serum, insulin, transferrin, lactoferrin, cholesterol, ethanolamine, sodium selenite, monothioglycerol, 2-mercaptoethanol, bovine serum albumin, sodium pyruvate, polyethylene glycol, various vitamins, various amino acids, agar, agarose, collagen, methylcellulose, various cytokines, various hormones, various proliferation factors, various extracellular matrices, various cell adhesion molecules and the like. Examples of the cytokine to be added to a medium include, but are not limited to, interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), interleukin-14 (IL-14), interleukin-15 (IL-15), interleukin-18 (IL-18), interleukin-21 (IL-21), interferon-α (IFN-α), interferon-β (IFN-β), interferon-γ (IFN-γ), granulocyte colony stimulating factor (G-CSF), monocyte colony stimulating factor (M-CSF), granulocyte-macrophage colony stimulating factor (GM-CSF), stem cell factor (SCF), flk2/flt3 ligand (FL), leukemia cell inhibitory factor (LIF), oncostatin M (OM), erythropoietin (EPO), thrombopoietin (TPO) and the like.

Examples of the hormone to be added to a medium include, but are not limited to, melatonin, serotonin, thyroxine, triiodothyronine, epinephrine, norepinephrine, dopamine, anti-Mullerian hormone, adiponectin, adrenocorticotropic hormone, angiotensinogen and angiotensin, antidiuretic hormone, atrial natriuretic peptide, calcitonin, cholecystokinin, corticotropin release hormone, erythropoietin, follicle stimulating hormone, gastrin, ghrelin, glucagon, gonadotropin release hormone, growth hormone release hormone, human chorionic gonadotropin, human placental lactogen, growth hormone, inhibin, insulin, insulin-like growth factor, leptin, luteinizing hormone, melanocyte stimulating hormone, oxytocin, parathyroid hormone, prolactin, secretin, somatostatin, thrombopoietin, thyroid-stimulating hormone, thyrotropin releasing hormone, cortisol, aldosterone, testosterone, dehydroepiandrosterone, androstenedione, dihydrotestosterone, estradiol, estrone, estriol, progesterone, calcitriol, calcidiol, prostaglandin, leukotriene, prostacyclin, thromboxane, prolactin releasing hormone, lipotropin, brain natriuretic peptide, neuropeptide Y, histamine, endothelin, pancreas polypeptide, rennin and enkephalin.

Examples of the growth factor to be added to a medium include, but are not limited to, transforming growth factor-α (TGF-α), transforming growth factor-β (TGF-β), macrophage inflammatory protein-la (MIP-la), epithelial cell growth factor (EGF), fibroblast growth factor-1, 2, 3, 4, 5, 6, 7, 8 or 9 (FGF-1, 2, 3, 4, 5, 6, 7, 8, 9), nerve cell growth factor (NGF) hepatocyte growth factor (HGF), leukemia inhibitory factor (LIF), protease nexin I, protease nexin II, platelet-derived growth factor (PDGF), choline vasoactive differentiation factor (CDF), chemokine, Notch ligand (Delta1 and the like), Wnt protein, angiopoietin-like protein 2, 3, 5 or 7 (Angpt2, 3, 5, 7), insulin like growth factor (IGF), insulin-like growth factor binding protein (IGFBP), Pleiotrophin and the like.

In addition, these cytokines and growth factors having amino acid sequences artificially altered by gene recombinant techniques can also be added. Examples thereof include IL-6/soluble IL-6 receptor complex, Hyper IL-6 (fusion protein of IL-6 and soluble IL-6 receptor) and the like.

Examples of the antibiotic to be added to a medium include Sulfonamides and preparations, penicillin, phenethicillin, methicillin, oxacillin, cloxacillin, dicloxacillin, flucloxacillin, nafcillin, ampicillin, penicillin, amoxicillin, ciclacillin, carbenicillin, ticarcillin, piperacillin, azlocillin, mezlocillin, mecillinam, andinocillin, cephalosporin and a derivative thereof, oxolinic acid, amifloxacin, temafloxacin, nalidixic acid, Piromidic acid, ciprofloxacin, cinoxacin, norfloxacin, perfloxacin, Rosaxacin, ofloxacin, enoxacin, pipemidic acid, sulbactam, clavulanic acid, β-bromopenisillanic acid, β-chloropenisillanic acid, 6-acetylmethylene-penisillanic acid, cephoxazole, sultampicillin, adinoshirin and sulbactam formaldehyde hudrate ester, tazobactam, aztreonam, sulfazethin, isosulfazethin, nocardicin, phenylacetamidophosphonic acid methyl, Chlortetracycline, oxytetracycline, tetracycline, demeclocycline, doxycycline, methacycline, and minocycline.

In one embodiment, supplements and serum replacements may be added to the medium. Examples thereof include, but are not limited to, StemPro (registered trademark) Neural Supplement (manufactured by Thermo Fisher), B-27 (registered trademark) Supplement (manufactured by Thermo Fisher), KnockOut (registered trademark) Serum Replacement (manufactured by Thermo Fisher), CTS (registered trademark) KnockOut (registered trademark) SR XenoFree Medium (manufactured by Thermo Fisher), ELAREM (registered trademark) Prime I Research Grade (manufactured by PL Bioscience), ELAREM (registered trademark) Perform I Research Grade or GMP Grade (manufactured by PL Bioscience), ELAREM (registered trademark) Perform-FD I Research Grade or GMP Grade (manufactured by PL Bioscience), ELAREM (registered trademark) Ultimate-FDi I GMP Grade (manufactured by PL Bioscience), Human Platelet Lysate Stemulate (registered trademark) (manufactured by Sexton Biotechnologies), Pathogen-Reduced Human Platelet Lysate nLiven PR (registered trademark) or T-Liven PR (registered trademark) (manufactured by Sexton Biotechnologies) UltraGRO (registered trademark) or -PURE GI or -Advanced GI or -PURE or -Advanced (manufactured by AventaCell BioMedical), Bio-Pure Human Serum Albumin (HSA) 10% solution (manufactured by Biological Industries), PLTMax (registered trademark) Human Platelet Lysate (manufactured by Biological Industries), PLTGold (registered trademark) Human Platelet Lysate (manufactured by Biological Industries), and the like.

Cell adhesion molecules that can be added to the medium include, but are not limited to, Vitronectin (VTN-N) Recombinant Human Protein, Truncated (manufactured by Thermo Fisher), CTS (registered trademark) Vitronectin (VTN-N) Recombinant Human Protein, Truncated (manufactured by Thermo Fisher), rhLaminin-521 (manufactured by Thermo Fisher), iMatrix-511 MG (manufactured by Matrixome), iMatrix-511 silk or -411 or -221 (manufactured by Matrixome), NutriCoat (registered trademark) Attachment Solution (manufactured by Biological Industries), and the like.

While the mixing ratio is not particularly limited, nanofiber dispersion:liquid medium (aqueous solution as medium) (volume ratio) is generally 1:99-99:1, preferably 10:90-90:10, more preferably, 20:80-80:20.

Suspending of cells in the present specification refers to a state where cells do not adhere to a culture container (non-adhesive), and whether the cell forms sediment is not questioned.

Suspension culture of adherent cells can be performed by culturing adherent cells while being attached to nanofibers optionally carrying extracellular matrix under stirring. A substrate composed of nanofibers optionally carrying extracellular matrix is dispersed without dissolving or attaching to a culture container in the liquid medium, and therefore, when adherent cells are stirring cultured in the liquid medium, the adherent cells attach to the substrate and are uniformly suspended in the medium.

When adherent cells are suspension cultured using a substrate composed of nanofibers optionally carrying extracellular matrix, adherent cells prepared separately are added to the medium composition containing the substrate and mixed uniformly. In this case, the mixing method is not particularly limited and, for example, manual mixing using pipetting and the like, mixing using instrument such as stirrer, vortex mixer, microplate mixer, shaking machine and the like can be mentioned.

After mixing the medium and the adherent cells, the obtained cell suspension is cultivated while stirring.

In the present invention, “stirring” refers to a state in which a substrate such as fibers and the like and cells are suspended in a medium, and the substrate and cells are continuously moved by an external force in the system. When the substrate and cells are brought into proper contact with each other in the medium by external force, the formation of a cell aggregate that embraces the substrate is promoted, and the cells can be efficiently proliferated. The external force applied to the system may be adjusted as appropriate depending on the substrate concentration, culture scale, and the like, and gentle mixing is preferred so as not to damage the cells. As a method of applying an external force,
(1) mixing by blades,
(2) shaking mixing such as reciprocal type, orbital type (e.g., embodiment where the culture container rotates horizontally), seesaw type, wave shaking type, and the like
(3) mixing by reflux or gas aeration,
(4) mixing by rotating in a roller bottle, or
(5) mixing by vibration using a vortex mixer and the like can be mentioned. The manner in which an external force is applied is not particularly limited as long as it is a mixed state in which application of an appropriate external force promotes uniform contact between the substrate and the cells, and as a result, the formation of cell aggregate that embraces the substrate is promoted.

As used herein, “uniform” refers to a suspended state from a macro perspective, where the substrate and cells are unevenly distributed on the bottom surface of the container and do not remain staying there. This does not mean that the substrate and cells are evenly distributed from a microscopic perspective on the entire culture medium.

A known method may be used to stir the liquid medium. Examples include, but are not limited to, magnetic stirrers, blades, and the like. In addition, the shape and number of blades for stirring, the rotating speed, and frequency can be appropriately set according to the purpose of those of ordinary skill in the art. The lower limit of the stirring speed (i.e., tip speed) used in the present invention is not particularly limited as long as the cells and substrate do not become still. For example, it is generally not less than 0.01 m/min, preferably not less than 0.10 m/min (e.g., not less than 0.15 m/min), more preferably not less than 0.90 m/min (e.g., 0.97 m/min). The upper limit thereof may be, for example, generally not more than 50.0 m/min, preferably not more than 30.0 m/min (e.g., 22.6 m/min), more preferably not more than 20.00 m/min (e.g., 15.08 m/min). In one embodiment of the present invention, the tip speed may be generally 0.01-50.0/min, preferably 0.10-30.0 m/min (e.g., 0.15-22.6 m/min), more preferably 0.90-16.00 m/min.

In one embodiment, the lower limit of the stirring speed (i.e., rotating speed) used in the present invention is not particularly limited as long as the cells and substrate do not become still. For example, it is generally not less than 1 rpm, preferably not less than 5 rpm, more preferably not less than 10 rpm. The upper limit thereof may be, for example, generally not more than 150 rpm, preferably not more than 140 rpm, more preferably not more than 120 rpm. In one embodiment of the present invention, the rotating speed of stirring may be generally 1-150 rpm, preferably 5-140 rpm, more preferably 10-120 rpm.

Moreover, the frequency of stirring may be any frequency as long as the desired effect of the present invention can be obtained. For example, one cycle may include stirring for 1 minute at a specific rotation speed selected from the aforementioned rotation speeds and no stirring for 59 minutes, and this cycle may be repeated during cell culture. Alternatively, stirring may be constantly performed during cell culture.

The temperature when cells are cultivated is generally 25 to 39° C., preferably 33 to 39° C. (e.g., 37° C.), for animal cells. The CO₂ concentration is generally 4 to 10% by volume in the culture atmosphere, and 4 to 6% volume is preferred. The level of oxygen dissolved in the medium can be appropriately set according to the cell type and the purpose of culture. Further, the pH when culturing cells may be appropriately set according to the cell type and the purpose of culture. In the case of animal cells, it is generally pH 7 to 8, preferably pH 7.2 to 7.8. In order to maintain the pH, it is also possible to adjust the amount and concentration of CO₂ to be added to the culture system or to add an acid or alkaline solution. Furthermore, it is also possible to culture by appropriately adding a cell nutrient source (e.g., glucose) or removing only waste products (e.g., lactic acid) using a membrane or the like. The culture period may be set as appropriately according to the object of the culture.

In the method of the present invention, adherent cells can be cultivated using culture vessels generally used for cell culture such as schale, flask, plastic bag, Teflon (registered trademark) bag, dish, schale, dish for tissue culture, multidish, microplate, microwell plate, multiplate, multiwell plate, chamber slide, tube, tray, culture bag, roller bottle and the like can be used for cultivation. These culture containers are desirably low cell-adhesive so that the adherent cells attached to the substrate used in the present invention will not adhere to the culture container. As a low cell—adhesive culture vessel, a culture vessel having a surface not artificially treated to improve adhesiveness to cells (e.g., coating treatment with extracellular matrix and the like), or a culture vessel having a surface artificially treated to reduce adhesiveness to cells can be used.

When the medium needs to be exchanged, the cells and substrate may be allowed to settle naturally by stopping stirring and only the supernatant is replaced. Alternatively, the cells are separated by centrifugation or filtration treatment, and a fresh medium can be added to the cells. Alternatively, the cells are appropriately concentrated by centrifugation or filtration treatment, and a fresh medium can be added to the concentrated liquid. For example, unlimitatively, the gravitational acceleration (G) of centrifugation is 100G to 400G, and the size of the pore of the filter used for the filtration treatment is 10 μm to 100 μm.

The adherent cells can also be cultured by automatically conducting cell seeding, medium exchange, cell image obtainment, and recovery of cultured cells, under a mechanical control and under a closed environment while controlling pH, temperature, oxygen concentration and the like and using a bioreactor and an automatic incubator capable of high-density culture.

When adherent cells are attached to a substrate composed of nanofibers optionally carrying extracellular matrix and cultured in suspension under conditions with stirring, the adherent cells efficiently proliferate in the form of spheres. Furthermore, when the adherent cells are stem cells such as mesenchymal stem cell and the like, the cells obtained by this method exhibit promoted gene expression of undifferentiation markers (OCT4, NANOG, etc.) and homing/migration markers (CXCR4, etc.). That is, the adherent cells (e.g., mesenchymal stem cells) obtained by the present invention may be suitable, for example, as cells for transplantation into a living body. In addition, the spheres obtained by the present invention tend to have a uniform size distribution.

When adherent cells are subjected to suspension culture while being attached to a substrate composed of nanofibers optionally carrying extracellular matrix to proliferate the adherent cells, a medium permitting proliferation of the adherent cells while maintaining the character thereof is used as the medium to be used for the suspension culture. Those of ordinary skill in the art can appropriately select the medium according to the type of the adherent cells.

In one embodiment, the medium used in the method of the present invention may contain chitosan nanofibers in addition to nanofibers optionally carrying extracellular matrix.

The chitosan nanofibers used in the method of the present invention can be those prepared according to the preparation method of nanofibers described above. Alternatively, commercially available chitosan nanofibers may also be used.

In one embodiment of the present invention, when only a nanofiber composed of water-insoluble polysaccharides is added to the liquid medium, the amount of the nanofiber composed of water-insoluble polysaccharides (e.g., chitin nanofiber) to be added to the medium is not particularly limited as long as the desired effect is obtained. It can be added to the liquid medium generally at 0.0001-0.2% (w/v), preferably 0.0005-0.1% (w/v), further preferably 0.001-0.07% (w/v), particularly preferably 0.003-0.05% (w/v).

In one embodiment of the method of the present invention, when a nanofiber composed of water-insoluble polysaccharides not carrying extracellular matrix and chitosan nanofiber are added to a liquid medium, to prepare a medium containing a nanofiber composed of water-insoluble polysaccharides (e.g., chitin nanofiber) and a chitosan nanofiber at a desired ratio (weight), they are blended at nanofiber composed of water-insoluble polysaccharides:chitosan nanofiber=1:0.01-10 (preferably nanofiber composed of water-insoluble polysaccharides:chitosan nanofiber=1:0.02-9, more preferably nanofiber composed of water-insoluble polysaccharides:chitosan nanofiber=1:0.05-8, further preferably nanofiber composed of water-insoluble polysaccharides:chitosan nanofiber=1:0.1-7, further more preferably nanofiber composed of water-insoluble polysaccharides:chitosan nanofiber=1:0.5-6, particularly preferably nanofiber composed of water-insoluble polysaccharides:chitosan nanofiber=1:1-5). The obtained mixture of nanofibers composed of water-insoluble polysaccharides/chitosan nanofiber can be blended in a liquid medium such that the concentration of the total nanofibers (nanofiber composed of water-insoluble polysaccharides and chitosan nanofiber) contained in the medium composition is generally 0.0001-0.2% (w/v), preferably 0.0005-0.1% (w/v), further preferably 0.001-0.07% (w/v), particularly preferably 0.003-0.05% (w/v). Alternatively, a desired medium may be prepared by separately adding the necessary amounts of nanofiber composed of water-insoluble polysaccharides (e.g., chitin nanofiber) and chitosan nanofiber to a liquid medium and stirring well.

In one embodiment, the concentrations of the nanofiber composed of water-insoluble polysaccharides (e.g., chitin nanofiber) and the chitosan nanofiber in the method of the present invention satisfy the following conditions:
(1) the concentration of the total nanofibers (nanofiber composed of water-insoluble polysaccharides and chitosan nanofiber) contained in the medium composition is 0.0001-0.2% (w/v), and the weight ratio of nanofiber composed of water-insoluble polysaccharides:chitosan nanofiber contained in the medium composition is 1:0.01-10 (preferably, 1:0.02-9, 1:0.05-8, 1:0.1-7, 1:0.5-7, or 1:1-6);
(2) the concentration of the total nanofibers (nanofiber composed of water-insoluble polysaccharides and chitosan nanofiber) contained in the medium composition is 0.0005-0.1% (w/v), and the weight ratio of nanofiber composed of water-insoluble polysaccharides:chitosan nanofiber contained in the medium composition is 1:0.01-10 (preferably, 1:0.02-9, 1:0.05-8, 1:0.1-7, 1:0.5-7, or 1:1-6);
(3) the concentration of the total nanofibers (nanofiber composed of water-insoluble polysaccharides and chitosan nanofiber) contained in the medium composition is 0.001-0.05% (w/v), and the weight ratio of nanofiber composed of water-insoluble polysaccharides:chitosan nanofiber contained in the medium composition is 1:0.01-10 (preferably, 1:0.02-9, 1:0.05-8, 1:0.1-7, 1:0.5-7, or 1:1-6); or
(4) the concentration of the total nanofibers (nanofiber composed of water-insoluble polysaccharides and chitosan nanofiber) contained in the medium composition is 0.003-0.05% (w/v), and the weight ratio of nanofiber composed of water-insoluble polysaccharides:chitosan nanofiber contained in the medium composition is 1:0.01-10 (preferably, 1:0.02-9, 1:0.05-8, 1:0.1-7, 1:0.5-7, or 1:1-6).

In another embodiment, the obtained mixture of the nanofiber composed of water-insoluble polysaccharides/chitosan nanofiber can be blended in a liquid medium such that the concentration of the total nanofibers (nanofiber composed of water-insoluble polysaccharides and chitosan nanofiber) contained in the medium is generally 0.0001-1.0% (w/v), preferably 0.001-0.5% (w/v), further preferably 0.002-0.3% (w/v), particularly preferably 0.003-0.1% (w/v).

In another embodiment, the concentrations of the nanofiber composed of water-insoluble polysaccharides (e.g., chitin nanofiber) and chitosan nanofiber in the method of the present invention satisfy the following conditions:
(5) the concentration of the total nanofibers (nanofiber composed of water-insoluble polysaccharides and chitosan nanofiber) contained in the medium composition is 0.0001-1.0% (w/v), and the weight ratio of nanofiber composed of water-insoluble polysaccharides:chitosan nanofiber contained in the medium composition is 1:0.01-10 (preferably, 1:0.02-9, 1:0.05-8, 1:0.1-7, 1:0.5-7, or 1:1-6);
(6) the concentration of the total nanofibers (nanofiber composed of water-insoluble polysaccharides and chitosan nanofiber) contained in the medium composition is 0.001-0.5% (w/v), and the weight ratio of nanofiber composed of water-insoluble polysaccharides:chitosan nanofiber contained in the medium composition is 1:0.01-10 (preferably, 1:0.02-9, 1:0.05-8, 1:0.1-7, 1:0.5-7, or 1:1-6);
(7) the concentration of the total nanofibers (nanofiber composed of water-insoluble polysaccharides and chitosan nanofiber) contained in the medium composition is 0.005-0.3% (w/v), and the weight ratio of nanofiber composed of water-insoluble polysaccharides:chitosan nanofiber contained in the medium composition is 1:0.01-10 (preferably, 1:0.02-9, 1:0.05-8, 1:0.1-7, 1:0.5-7, or 1:1-6); or
(8) the concentration of the total nanofibers (nanofiber composed of water-insoluble polysaccharides and chitosan nanofiber) contained in the medium composition is 0.01-0.1% (w/v), and the weight ratio of nanofiber composed of water-insoluble polysaccharides:chitosan nanofiber contained in the medium composition is 1:0.01-10 (preferably, 1:0.02-9, 1:0.05-8, 1:0.1-7, 1:0.5-7, or 1:1-6).

In the method of the present invention, when a nanofiber composed of water-insoluble polysaccharides carrying extracellular matrix and chitosan nanofiber are added to a liquid medium, to prepare a medium containing a nanofiber (e.g., chitin nanofiber) carrying extracellular matrix (e.g., vitronectin) and a chitosan nanofiber at a desired ratio (weight), they are blended at nanofiber carrying extracellular matrix:chitosan nanofiber=1:0.01-10 (preferably nanofiber carrying extracellular matrix:chitosan nanofiber=1:0.02-9, more preferably nanofiber carrying extracellular matrix:chitosan nanofiber=1:0.05-8, further preferably nanofiber carrying extracellular matrix:chitosan nanofiber=1:0.1-7, further more preferably nanofiber carrying extracellular matrix:chitosan nanofiber=1:0.5-6, particularly preferably nanofiber carrying extracellular matrix:chitosan nanofiber=1:1-5). The obtained mixture of nanofibers carrying extracellular matrix/chitosan nanofibers can be blended in a liquid medium such that the concentration of the total nanofibers (nanofiber carrying extracellular matrix and chitosan nanofiber) contained in the medium composition is generally 0.0001-0.2% (w/v), preferably 0.0005-0.1% (w/v), further preferably 0.001-0.07% (w/v), particularly preferably 0.003-0.05% (w/v). Alternatively, a desired medium may be prepared by separately adding the necessary amounts of nanofiber (e.g., chitin nanofiber) carrying extracellular matrix (e.g., vitronectin), and chitosan nanofiber to a liquid medium and stirring well.

In one embodiment, the concentrations of the nanofiber (e.g., chitin nanofiber) carrying extracellular matrix (e.g., vitronectin) and the chitosan nanofiber in the method of the present invention satisfy the following conditions:
(1) the concentration of the total nanofibers (nanofiber carrying extracellular matrix and chitosan nanofiber) contained in the medium composition is 0.0001-0.2% (w/v), and the weight ratio of nanofiber carrying extracellular matrix:chitosan nanofiber contained in the medium composition is 1:0.01-10 (preferably, 1:0.02-9, 1:0.05-8, 1:0.1-7, 1:0.5-7, or 1:1-6);
(2) the concentration of the total nanofibers (nanofiber carrying extracellular matrix and chitosan nanofiber) contained in the medium composition is 0.0005-0.1% (w/v), and the weight ratio of nanofiber carrying extracellular matrix:chitosan nanofiber contained in the medium composition is 1:0.01-10 (preferably, 1:0.02-9, 1:0.05-8, 1:0.1-7, 1:0.5-7, or 1:1-6);
(3) the concentration of the total nanofibers (nanofiber carrying extracellular matrix and chitosan nanofiber) contained in the medium composition is 0.001-0.05% (w/v), and the weight ratio of nanofiber carrying extracellular matrix:chitosan nanofiber contained in the medium composition is 1:0.01-10 (preferably, 1:0.02-9, 1:0.05-8, 1:0.1-7, 1:0.5-7, or 1:1-6); or
(4) the concentration of the total nanofibers (nanofiber carrying extracellular matrix and chitosan nanofiber) contained in the medium composition is 0.003-0.05 (w/v), and the weight ratio of nanofiber carrying extracellular matrix:chitosan nanofiber contained in the medium composition is 1:0.01-10 (preferably, 1:0.02-9, 1:0.05-8, 1:0.1-7, 1:0.5-7, or 1:1-6).

In another embodiment, the obtained mixture of nanofiber carrying extracellular matrix/chitosan nanofiber can be blended in a liquid medium such that the concentration of the total nanofibers (nanofiber carrying extracellular matrix and chitosan nanofiber) contained in the medium composition is generally 0.0001-1.0% (w/v), preferably 0.001-0.5% (w/v), further preferably 0.002-0.3% (w/v), particularly preferably 0.003-0.1% (w/v).

In another embodiment, the concentrations of the nanofiber (e.g., chitin nanofiber) carrying extracellular matrix (e.g., vitronectin) and the chitosan nanofiber in the method of the present invention satisfy the following conditions:
(5) the concentration of the total nanofibers (nanofiber carrying extracellular matrix and chitosan nanofiber) contained in the medium composition is 0.0001-1.0% (w/v), and the weight ratio of nanofiber carrying extracellular matrix:chitosan nanofiber contained in the medium composition is 1:0.01-10 (preferably, 1:0.02-9, 1:0.05-8, 1:0.1-7, 1:0.5-7, or 1:1-6);
(6) the concentration of the total nanofibers (nanofiber carrying extracellular matrix and chitosan nanofiber) contained in the medium composition is 0.001-0.5% (w/v), and the weight ratio of nanofiber carrying extracellular matrix:chitosan nanofiber contained in the medium composition is 1:0.01-10 (preferably, 1:0.02-9, 1:0.05-8, 1:0.1-7, 1:0.5-7, or 1:1-6);
(7) the concentration of the total nanofibers (nanofiber carrying extracellular matrix and chitosan nanofiber) contained in the medium composition is 0.005-0.3% (w/v), and the weight ratio of nanofiber carrying extracellular matrix:chitosan nanofiber contained in the medium composition is 1:0.01-10 (preferably, 1:0.02-9, 1:0.05-8, 1:0.1-7, 1:0.5-7, or 1:1-6); or
(8) the concentration of the total nanofibers (nanofiber carrying extracellular matrix and chitosan nanofiber) contained in the medium composition is 0.01-0.1 (w/v), and the weight ratio of nanofiber carrying extracellular matrix:chitosan nanofiber contained in the medium composition is 1:0.01-10 (preferably, 1:0.02-9, 1:0.05-8, 1:0.1-7, 1:0.5-7, or 1:1-6).

In one embodiment, polysaccharides that have the effect of suspending cells and tissues can also be used in combination. Examples of the polysaccharides include, but are not limited to, hyaluronic acid, gellan gum, deacylated gellan gum, rhamsan gum, diutan gum, xanthan gum, carrageenan, zanthan gum, hexuronic acid, fucoidan, pectin, pectic acid, pectinic acid, heparan sulfate, heparin, heparitin sulfate, keratosulfate, chondroitin sulfuric acid, dermatan sulfuric acid, rhamnan sulfate and salts thereof. These polysaccharides may be used alone or in combination of two or more.

2. Method for Producing Adherent Cell Spheres with Uniform Sphere Size

The present invention also provides a method for producing spheres of adherent cells having a uniform sphere size, the method including a step of culturing adherent cells in suspension in a medium containing nanofibers composed of water-insoluble polysaccharides, in which the culture is performed along with stirring (hereinafter sometimes referred to as “the production method of the present invention”). Uniformity of sphere size can be important, for example, from the viewpoint of affording a uniform quality of spheroid preparations.

The production method of the present invention is characterized in that it includes a nanofiber composed of water-insoluble polysaccharides. The production method of the present invention took note of the uniformity of the spheres prepared by the method of the present invention. Accordingly, the production method of the present invention and the method of the present invention are identical in the constitution. Therefore, in each correspondence of the production method of the present invention, the constitution explained in the method of the present invention can be used. For example, a nanofiber composed of water-insoluble polysaccharides, a chitosan nanofiber, an extracellular matrix, and the like in the production method of the present invention are the same as those described in the method of the present invention.

### 3. Isolation Method of Spheres

The present invention also provides a method for isolating spheres, including a step of applying a suspension of spheres prepared by the production method of the present invention to a cell strainer (hereinafter sometimes to be referred to as “the isolation method of the present invention”).

The pore size of the mesh of a cell strainer used in the isolation method of the present invention is not particularly limited as long as it is smaller than the size of the spheres to be recovered, and may be generally 20-600 μm, preferably, 20-550 μm, 20-500 μm, 20-450 μm, 20-400 μm, 20-350 μm, more preferably, 30-350 μm, 30-300 μm, 30-280 μm, 30-250 μm, 30-230 μm, particularly preferably, 50-250 μm, 60-250 μm, 60-230 μm, 60-220 μm.

The cell strainer used in the isolation method of the present invention may be a commercially available product. In one embodiment, the cell strainer manufactured by PluriSelect and used in the following Examples may be favorably used, but is not particularly limited. For scale-up, Harvestainer (manufactured by Thermo Fisher Scientific), which is a large bag-shaped cell strainer, or a device with similar functions, or CTS Rotea Counterflow Centrifugation System (manufactured by Thermo Fisher Scientific) or Ksep (registered trademark) Systems (manufactured by Sartorius), which are continuous elutriation systems that can separate spheres with the desired size based on the size and specific gravity, can be used.

The conditions for passing a sphere suspension through the cell strainer are not particularly limited, and may be performed using a method known per se or following instructions provided by cell strainer manufacturers.

The spheres prepared by the production method of the present invention are mixtures with a nanofiber composed of water-insoluble polysaccharides and the like. By using the isolation method of the present invention, spheres can be efficiently isolated from the mixture.

4. Method for Dispersing Spheres into Single Cells

The present invention also provides a method for dispersing adherent cells in the form of spheres into single cells, including
a first step of suspension culturing adherent cells in a medium containing nanofibers composed of water-insoluble polysaccharides, and
a second step of treating the spheres of the adherent cells obtained in the first step with a cell dispersing agent (hereinafter sometimes to be referred to as “the single cell dispersion method of the present invention”).

A nanofiber composed of water-insoluble polysaccharides to be used in the first step of the single cell dispersion method of the present invention is the same as that described in the method of the present invention.

In the first step of the single cell dispersion method of the present invention, the suspension culture of the adherent cells may be performed under static conditions or stirring conditions. When performing under stirring conditions, the various parameters described in the method of the present invention may be adopted as appropriate.

In one embodiment, in the first step of the single cell dispersion method of the present invention, the nanofiber composed of water-insoluble polysaccharides can carry an extracellular matrix. Extracellular matrix, the amount thereof to be carried, and the like are the same as those described in the method of the present invention.

In one embodiment, in the first step of the single cell dispersion method of the present invention, chitosan nanofibers can be further added to the medium. The amount of chitosan nanofiber to be used is the same as that described in the method of the present invention.

In addition, various conditions regarding the suspension culture of adherent cells in the single cell dispersion method of the present invention (species of adhesive cells, culture containers, components that may be added, etc.) are the same as those described in the method of the present invention.

The cell dispersing agent that can be used in the single cell dispersion method of the present invention is not particularly limited as long as it can disperse adherent cell spheres. As one embodiment of such a cell dispersing agent, an enzyme having an action of dispersing cells or an enzyme decomposing an extracellular matrix, such as trypsin, collagenase, dispase, thermolysin, papain, hyaluronidase, elastase, pronase, and the like can be used. Furthermore, a chelating agent such as EDTA may be used as a cell dispersing agent. In addition, as the cell dispersing agent, a plurality of enzymes may be used in combination as a cocktail, or an enzyme and a chelating agent may be used in combination. Furthermore, enzymes that decompose nanofibers, such as chitinase and lysozyme, and fine particles such as silica, which are additives that promote the decomposition reaction, can also be used—in combination. The amount and concentration of the cell dispersing agent and chelating agent may be adjusted as appropriate. When the spheres are large and difficult to disperse, the amount of enzyme to be added or the concentration may be increased. The cell dispersing agent can be prepared by a method known per se, or a commercially available one can also be used. Commercially available cell dispersing agents include, but are not limited to, Liberase (registered trademark) TM, TL, DL, DH, TH (manufactured by Merck), Liberase MNP-S, Liberase MTF C/T, Liberase T-Flex (manufactured by Roche Diagnostics), TrypLE Select Enzyme (manufactured by Thermo Fisher Scientific), HyQTase enzymatic cell detachment solution (manufactured by Cytiva), Accutase (registered trademark), Accumax (registered trademark), AccutaseLZ (registered trademark) (manufactured by Innovative Cell Technologies), ReLeSR (registered trademark), Gentle Cell Dissociation Reagent (manufactured by STEMCELL Technologies), ZymeFree (registered trademark) Enzyme Free Cell Dissociation Reagent (manufactured by HiMedia Laboratories), Collagenase, Collagenase/Elastase, Collagenase, Type1-7, STEMxyme (registered trademark) 1, STEMxyme (registered trademark) 2, Collagenase, Type A-C, Neutral Protease (Dispase), Elastase (manufactured by Worthington biochemical corporation), Dispase (manufactured by Thermo Fisher Scientific), BD Horizon (registered trademark) Dri Tumor & Tissue Dissociation Reagent (TTDR) (manufactured by BD), Chitinase 18a (manufactured by nzytech), Chitinase 18a from Bacillus licheniformis, Recombinant (manufactured by Creative Enzymes), Chitinase (Clostridium thermocellum) (manufactured by Megazyme), Lysozyme, from Egg White (manufactured by FUJIFILM Wako Pure Chemical), Japanese Pharmacopoeia Lysozyme RS (manufactured by FUJIFILM Wako Pure Chemical), and the like.

When treating spheres of adherent cells with a cell dispersion agent, those skilled in the art can appropriately determine various conditions (treatment temperature, treatment time, etc.) according to the kind of cell dispersing agent to be used. The treatment time may be generally 5 sec-60 min, preferably 10 sec-50 min, 30 sec-40 min, further preferably 1 min-30 min. The treatment temperature may be generally 0-70° C., preferably 5-50° C., further preferably 10-40° C.

For example, a ROCK inhibitor such as Y-27632 or the like and DNaseI and the like can be added as appropriate in combination with a cell dispersing agent during single cell dispersion, or after single cell dispersion, in order to suppress adverse effects on cells (e.g., cell death or viscosity increase) caused by dispersion of cells.

In one embodiment, the single cell dispersion method of the present invention may be a method for dispersing adherent cells in the form of spheres into single cells, including a step of treating the spheres of the adherent cells obtained by the aforementioned method of the present invention, the production method of the present invention, or the isolation method of the present invention with a cell dispersing agent.

The single cell dispersion step is not particularly limited as long as the cells are in a viable and single cell state, and may be performed in a stationary state or in a stirred state, or may be performed in a culture bag or a bioreactor. In addition, cells can be dispersed into single cells using the “cell dispersion tool” (manufactured by ABLE Corporation), which is a device for dispersing cells into single cells. Also, cells can be dispersed into single cells by circulating in the flow path of CTS Rotea Counterflow Centrifugation System (manufactured by Thermo Fisher Scientific) or Ksep (registered trademark) Systems (manufactured by Sartorius).

While not bound by theory, adherent cell spheres prepared by a suspension culture method that does not use a substrate such as nanofiber and the like are cell aggregates consisting only of adherent cells. In this cell aggregate, since the cells are strongly adhered to each other, it is considered necessary to perform a highly strong dispersion treatment in order to disperse the cell aggregate into single cells. However, the highly strong dispersion treatment damages the cells, and the number of viable cells resulting from single cell dispersion decreases. On the other hand, a cell aggregate composed of adhesive cells and a substrate such as nanofiber and the like may permit a comparatively mild dispersion treatment. As a result, it is considered that adherent cells efficiently dispersed into single cells can be prepared.

5. Mesenchymal Stem Cells with Promoted Expression of Specific Genes

The present invention also provides a mesenchymal stem cell in which expression of a specific gene is promoted as compared with that in a mesenchymal stem cell cultured by adhesion culture (hereinafter sometimes referred to as “the mesenchymal stem cell of the present invention”). The mesenchymal stem cell of the present invention can be prepared by culturing mesenchymal stem cells using the aforementioned method of the present invention. When cultured in suspension using nanofibers composed of water-insoluble polysaccharides and the like under stirring conditions, the mesenchymal stem cell of the present invention becomes a mesenchymal stem cell with gene expression profiles different from those of mesenchymal stem cells subjected to adhesion culture.

Genes whose expression is promoted in the mesenchymal stem cell of the present invention include CD55 (NCBI Gene ID:1604), HMOX1 (NCBI Gene ID:3162), TSPAN7 (NCBI Gene ID:7102), RAB27B (NCBI Gene ID:5874), IL33 (NCBI Gene ID:90865), GPX3 (NCBI Gene ID:2878), and MFAP4 (NCBI Gene ID:4239).

The gene whose expression is enhanced is at least one gene selected from the group consisting of CD55, HMOX1, TSPAN7, RAB27B, IL33, GPX3, and MFAP4, preferably at least 2, at least 3, or at least 4 of these genes, further preferably at least 5, at least 6, or at least 7 of these genes, and particularly preferably all of these genes are promoted.

As to the expression level of the specific gene in the mesenchymal stem cell of the present invention, the expression may be promoted, but not limited to, generally 1.1 times or more, preferably 1.2 times or more, 1.3 times or more, 1.4 times or more, 1.5 times or more, 1.6 times or more, 1.7 times or more, 1.8 times or more, 1.9 times or more, 2.0 times or more, 2.5 times or more, 3.0 times or more, 3.5 times or more, 4.0 times or more, 4.5 times or more, 5.0 times or more, 5.5 times or more, 6.0 times or more, 6.5 times or more, 7.0 times or more, 7.5 times or more, 8.0 times or more, 8.5 times or more, 9.0 times or more, 9.5 times or more, or 10.0 times or more, as compared with the expression level of the specific gene in mesenchymal stem cells cultured by adherent culture as a control.

The culture conditions for mesenchymal stem cells to be used as a control are not particularly limited as long as the mesenchymal stem cells can be maintained and/or proliferated under adhesion conditions. As one embodiment, the conditions used in the Examples of the present application (medium: mesenchymal stem cell growth medium 2 (manufactured by PromoCell, #C-28009), container: 10 cm dish (manufactured by Corning, #430167), temperature: 37° C., CO₂ concentration: 5%) can be mentioned, but are not limited to these.

In one embodiment, the condition for the adhesion culture of mesenchymal stem cells is two-dimensional culture using a culture dish.

Whether the expression of these genes is promoted can be determined by a method known per se. For example, as shown in Examples below, a method using real-time PCR is exemplified, but the method is not limited thereto.

In a preferred embodiment of the mesenchymal stem cell of the present invention, the mesenchymal stem cell of the present invention characteristically shows a promoted expression level of at least one protein selected from the group consisting of PGE2, RAB27B, NFE2 L2 (also referred to as “NRF2”), P65, and p-P65 (phosphorylated form of P65). In one embodiment, the mesenchymal stem cell of the present invention shows promoted expression levels of any two of PGE2, RAB27B, NFE2 L2, P65, and p-P65. In one embodiment, the mesenchymal stem cell of the present invention shows promoted expression levels of any three of PGE2, RAB27B, NFE2 L2, P65, and p-P65. In one embodiment, the mesenchymal stem cell of the present invention shows promoted expression levels of any four of PGE2, RAB27B, NFE2 L2, P65, and p-P65. In one embodiment, the mesenchymal stem cell of the present invention shows promoted expression levels of all of PGE2, RAB27B, NFE2 L2, P65, and p-P65.

As to the protein expression level of RAB27B, NFE2 L2, P65, and/or p-P65 in the mesenchymal stem cell of the present invention, the expression may be promoted, but not limited to, generally 1.1 times or more, preferably 1.2 times or more, 1.3 times or more, 1.4 times or more, 1.5 times or more, 1.6 times or more, 1.7 times or more, 1.8 times or more, 1.9 times or more, 2.0 times or more, 2.5 times or more, 3.0 times or more, 3.5 times or more, 4.0 times or more, 4.5 times or more, 5.0 times or more, 5.5 times or more, 6.0 times or more, 6.5 times or more, 7.0 times or more, 7.5 times or more, 8.0 times or more, 8.5 times or more, 9.0 times or more, 9.5 times or more, or 10.0 times or more, as compared with the expression level of the protein in mesenchymal stem cells cultured by adherent culture as a control.

Whether the expression level of these proteins is promoted can be determined by a method known per se. For example, as shown in Examples below, a method using Western blotting method and a method using ELISA method are exemplified, but the method is not limited thereto.

The tissue from which the mesenchymal stem cell of the present invention is derived is not particularly limited, and the mesenchymal stem cell may be derived from any tissue. For example, the mesenchymal stem cell of the invention can be derived from umbilical cord, bone marrow, adipose tissue, or peripheral blood. Preferably, the mesenchymal stem cell of the present invention is derived from the umbilical cord, bone marrow, or adipose tissue, more preferably from the umbilical cord or adipose tissue, particularly preferably from adipose tissue.

The mesenchymal stem cell of the present invention shows promoted production of extracellular vesicles as compared with the mesenchymal stem cells cultured by adherent culture.

Extracellular vesicles (EVs) are vesicles formed of a lipid bilayer membrane. Extracellular vesicles are classified into exosomes, microvesicles, and apoptotic bodies mainly based on differences in the formation mechanisms. In one embodiment of the present invention, the extracellular vesicle is an exosome.

Exosomes encapsulate a “load” (e.g., mRNA, miRNA, protein, lipid, etc.). It is known that the amount and kind of these loads vary depending on the state of the cell that secretes the exosome. Therefore, the development of the techniques for detecting diseases based on exosome analysis and the development of the methods for treating diseases that use exosome as a therapeutic target are in progress.

It has been reported that exosomes are secreted from various types of cells. In particular, it has been reported that exosomes secreted from mesenchymal stem cells have interesting properties. Application of mesenchymal stem cells to regenerative medicine is progressing because they have the ability to differentiate into various cells and have a low risk of tumor formation. Here, it is suggested that the therapeutic effect of the transplantation of mesenchymal stem cells depends on humoral factors such as mRNAs, miRNAs, proteins, and lipids contained in exosomes derived from transplanted mesenchymal stem cells (Spees J L et al., Stem Cell Res Ther. 2016 Aug. 31; 7(1):125.). Therefore, studies are ongoing as to the use of exosomes derived from mesenchymal stem cells as therapeutic agents. For example, it has been reported that exosomes derived from mesenchymal stem cells suppress tissue fibrosis in liver diseases and kidney diseases (Kan Yin et al., Biomark Res. 2019 Apr. 4; 7:8), and also have therapeutic effects on cardiac disease, Alzheimer's disease, and the like (Matthew H Forsberg et al. Front Cell Dev Biol. 2020 Jul. 17; 8:665.). Therefore, the mesenchymal stem cell of the present invention with an enhanced ability to produce exosomes may be used as therapeutic or prophylactic medicaments for various diseases.

In one embodiment, the production amount of extracellular vesicle in the mesenchymal stem cell of the present invention is, but not limited to, generally 1.1 times or more, preferably 1.2 times or more, 1.3 times or more, 1.4 times or more, 1.5 times or more, 1.6 times or more, 1.7 times or more, 1.8 times or more, 1.9 times or more, 2.0 times or more, 2.5 times or more, 3.0 times or more, 3.5 times or more, 4.0 times or more, 4.5 times or more, 5.0 times or more, 5.5 times or more, 6.0 times or more, 6.5 times or more, 7.0 times or more, 7.5 times or more, 8.0 times or more, 8.5 times or more, 9.0 times or more, 9.5 times or more, or 10.0 times or more, as compared with the production amount of the extracellular vesicle in mesenchymal stem cells cultured by adherent culture as a control.

In another embodiment, the production amount of exosome in the mesenchymal stem cell of the present invention is, but not limited to, generally 1.1 times or more, preferably 1.2 times or more, 1.3 times or more, 1.4 times or more, 1.5 times or more, 1.6 times or more, 1.7 times or more, 1.8 times or more, 1.9 times or more, 2.0 times or more, 2.5 times or more, 3.0 times or more, 3.5 times or more, 4.0 times or more, 4.5 times or more, 5.0 times or more, 5.5 times or more, 6.0 times or more, 6.5 times or more, 7.0 times or more, 7.5 times or more, 8.0 times or more, 8.5 times or more, 9.0 times or more, 9.5 times or more, or 10.0 times or more, as compared with the production amount of the exosome in mesenchymal stem cells cultured by adherent culture as a control.

As shown in Examples below, the mesenchymal stem cell of the present invention characteristically shows promoted production of “PGE2” as compared with mesenchymal stem cells prepared by adhesion culture. It is known that PGE2 is one of the secreted proteins that have anti-inflammatory action. Therefore, the mesenchymal stem cell of the present invention can be favorably used as an anti-inflammatory agent.

### 6. Method for Enhancing Production of Extracellular Vesicle of Mesenchymal Stem Cell

The present invention also provides a method for enhancing production of an extracellular vesicle of a mesenchymal stem cell, including a step of suspension culturing the mesenchymal stem cell in a medium containing a nanofiber composed of water-insoluble polysaccharides, wherein the culture is performed along with stirring (hereinafter sometimes referred to as “the enhancement method of the present invention”).

The practice of the enhancement method of the present invention is synonymous with culturing mesenchymal stem cells by the method of the present invention. Therefore, various conditions in the enhancement method of the present invention are the same as those described in the method of the present invention. Moreover, the mesenchymal stem cell of the present invention can be obtained by culturing mesenchymal stem cell using the enhancement method of the present invention. The enhancement method of the present invention can also be described as a method for producing a mesenchymal stem cell in which production of an extracellular vesicle is enhanced.

### 7. Agent for Treating Inflammatory Diseases, Containing Mesenchymal Stem Cells of the Present Invention

The present invention also provides an agent for treating inflammatory diseases, containing the mesenchymal stem cell of the present invention (hereinafter sometimes referred to as “the therapeutic agent for inflammatory diseases of the present invention”).

As described above, the mesenchymal stem cell of the present invention shows promoted secretion of PGE2 having an anti-inflammatory action. Therefore, the mesenchymal stem cell of the present invention can be extremely useful as a therapeutic agent for inflammatory diseases.

The amount of the mesenchymal stem cell of the present invention contained in the therapeutic agent for inflammatory diseases of the present invention is not particularly limited, and may be generally 0.001% by weight or more, preferably 0.01% by weight or more, 0.05% by weight or more, 0.1% by weight or more, or 0.5% by weight or more, more preferably 1% by weight or more, based on the total weight of the agent. In addition, the upper limit is not particularly limited, and may be generally 100% by weight or less, preferably 90% by weight or less, 70% by weight or less, 50% by weight or less, or 30% by weight or less, more preferably 10% by weight or less. In one embodiment, the amount of the mesenchymal stem cell of the present invention contained in the anti-inflammatory agent of the present invention is generally 0.001-100% by weight, preferably 0.01-90% by weight, 0.05-70% by weight, 0.1-50% by weight, or 0.5-30% by weight, more preferably 1-10% by weight, but is not limited thereto.

The therapeutic agent for inflammatory diseases of the present invention may contain components other than the mesenchymal stem cell of the present invention. The other components may include, for example, pharmaceutically acceptable additives for pharmaceutical products. The additives for pharmaceutical products include, but are not limited to, isotonizing agent, buffering agent, pH adjuster, stabilizer, chelating agent, antiseptic, and the like.

Examples of the isotonizing agent include sodium chloride, potassium chloride, saccharides, glycerol, and the like. Examples of the buffering agent include boric acid, phosphoric acid, acetic acid, citric acid, and salts corresponding thereto (e.g., alkali metal salts and alkaline earth metal salts thereof such as sodium salt, potassium salt, calcium salt, magnesium salt, and the like), and the like. Examples of the pH adjuster include inorganic acids such as hydrochloric acid, sulfuric acid, phosphoric acid, polyphosphoric acid, boric acid, borax, and the like; organic acids such as acetic acid, propionic acid, oxalic acid, gluconic acid, fumaric acid, lactic acid, citric acid, succinic acid, tartaric acid, malic acid, and the like; inorganic bases such as potassium hydroxide, sodium hydroxide, and the like; organic bases such as monoethanolamine, triethanolamine, diisopropanol amine, triisopropanol amine, and the like; ammonium acetate, sodium lactate, sodium citrate, potassium carbonate, sodium hydrogen carbonate, sodium carbonate, ammonium hydrogencarbonate, dipotassium phosphate, potassium dihydrogenphosphate, sodium hydrogenphosphate, sodium dihydrogen phosphate, calcium lactate, and the like. Examples of the stabilizer include human serum albumin, general L-amino acid, saccharides, cellulose derivative, and the like. These can be used alone or in combination with surfactants and the like. The above-mentioned L-amino acid may be any of glycine, cysteine, glutamic acid, and the like and is not limited to these. Saccharides may be any of monosaccharides such as glucose, mannose, galactose, fructose, and the like, sugar alcohols such as mannitol, inositol, xylitol, and the like, disaccharides such as sucrose, maltose, lactose, and the like, polysaccharides such as dextran, hydroxypropylstarch, chondroitin sulfuric acid, hyaluronic acid, and the like, derivative thereof, and the like, and are not limited to these. Cellulose derivative may be any of methyl cellulose, ethyl cellulose, hydroxyethyl cellulose, hydroxypropyl cellulose, hydroxypropylmethyl cellulose, sodium carboxymethyl cellulose, and the like, and are not limited to these. Examples of the chelating agent include sodium edetate, citric acid, and the like.

The form of the therapeutic agent for inflammatory diseases of the present invention is not particularly limited as long as it permits parenteral administration to a subject. For example, it can be a liquid form composed of cells and a suitable dispersion medium. In addition, when the therapeutic agent for inflammatory diseases of the present invention is directly applied to a seriously ill part, the therapeutic agent for inflammatory diseases of the present invention may also be formed into a sheet in which mesenchymal stem cells are adhered to a biocompatible material.

The amount of the therapeutic agent for inflammatory diseases of the present invention to be administered to a subject is not particularly limited, and may be any amount that can reduce the inflammatory response. Such an amount can be appropriately determined in consideration of the degree of inflammation, the age and body weight of the subject, administration method, number of administrations, form of the therapeutic agent for inflammatory diseases of the present invention, and the like.

In one embodiment, the therapeutic agent for inflammatory diseases of the present invention is applied to subjects suffering from inflammatory diseases. Examples of the inflammatory diseases include, but are not limited to, inflammatory bowel disease, ulcerative colitis, Crohn's disease, nephritis, acute nephritis, chronic nephritis, glomerulonephritis, IgA nephropathy, diabetic nephropathy, membranous nephropathy, hydronephrosis, contrast nephropathy, pyelonephritis, renal failure, interstitial nephritis, renal disorder, nephrotic syndrome, hypertensive nephrosclerosis, diabetic glomerulosclerosis, renal calculi, amyloid kidney, renal vein thrombosis, Alport syndrome, hepatitis, cirrhosis, pancreatitis, pneumonia, sinusitis, rhinitis, arthritis (arthropathy), knee osteoarthritis, hand osteoarthritis, ankle osteoarthritis, hip osteoarthritis, rheumatoid arthritis, periodic fever, aphthous stomatitis, pharyngitis and cervical adenitis syndrome (PFAPA), Adult-onset Still's disease, Behcet's disease, gout, pseudogout, Schnitzler syndrome, chronic recurrent multifocal osteomyelitis (CRMO), Cryopyrin associated periodic syndrome (CAPS), familial cold urticaria, Muckle-Wells syndrome, Chronic infantile neurologic cutaneous, and articular syndrome (CINCA syndrome)/Neonatal onset multisystem inflammatory disease (NOMID), TNF (Tumor Necrosis Factor) receptor-associated periodic syndrome (TRAPS), hyper IgD syndrome (Mevalonate Kinase Deficiency), Blau syndrome/early-onset sarcoidosis, familial Mediterranean fever, PAPA (pyogenic arthritis, pyoderma gangrenosum and Acne) syndrome, Nakajo-Nishimura syndrome, Majeed syndrome, NLRP12-associated periodic syndrome (NAPS12), Interleukin 1 receptor antagonist deficiency (DIRA), Interleukin 36 receptor antagonist deficiency (DITRA), PLCG2-associated antibody deficiency and immune dysregulation (PLAID), HOIL-1 deficiency, SLC29A3 deficiency, CARD14 abnormality, ADA2 (adenosine deaminase 2) deficiency, STING-Associated Vasculopathy with Onset in Infancy (SAVI), and NLRC4 abnormality, and the like. In one embodiment, the disease to which the anti-inflammatory agent of the present invention is favorably applied may be arthropathy, particularly, knee osteoarthritis, hand osteoarthritis, ankle osteoarthritis, or hip osteoarthritis.

Furthermore, the subject to which the therapeutic agent for inflammatory diseases of the present invention is applied is not particularly limited as long as it is an organism that can suffer from inflammatory diseases. It is generally a mammal such as rat, mouse, rabbit, guinea pig, squirrel, hamster, vole, platypus, dolphin, whale, dog, cat, goat, bovine, horse, sheep, swine, elephant, common marmoset, squirrel monkey, Macaca mulatta, chimpanzee, human, or the like, preferably human.

The mesenchymal stem cells of the present invention contained in the anti-inflammatory agent of the present invention may be spheres, single cells, or a mixture thereof. In one embodiment, the mesenchymal stem cells of the present invention contained in the anti-inflammatory agent of the present invention may be single cells.

By administering the anti-inflammatory agent of the present invention to a subject, the inflammatory disease of the subject can be treated. Therefore, the present invention provides a method for treating inflammatory diseases in a subject.

The present invention is explained more specifically in the following Examples. However, the present invention is not limited in any way by the Examples.

### EXAMPLE

[Preparation Example 1] Preparation of substrate An aqueous dispersion containing chitin nanofibers carrying vitronectin and chitosan nanofibers was prepared according to the description in WO2015/111686 and WO2021/002448. Specifically, it was prepared as follows. A 2% by mass chitin nanofiber aqueous dispersion prepared according to the description in WO2015/111686 was sterilized in an autoclave at 121° C. for 20 min. Thereafter, this aqueous dispersion was mixed and suspended in sterile distilled water (Otsuka Distilled Water, manufactured by Otsuka Pharmaceutical Factory Co., Ltd.) to a concentration of 1% (w/v) to produce a sterile aqueous dispersion containing chitin nanofibers. To a 1% (w/v) chitin nanofiber aqueous dispersion (5 mL) was added with a vitronectin aqueous solution containing 500 μg/mL (Gibco Vitronectin (VTN-N) Recombinant Human Protein, Truncated, manufactured by Thermo Fisher Scientific) (0.5 mL), mixed by pipetting, and then stored by standing at 4° C. overnight to produce an aqueous dispersion containing chitin nanofibers carrying vitronectin. When analyzed according to the description in WO2021/002448, the amount of the vitronectin carried was 20 μg/mL (2.2 mg of vitronectin per 1 g of chitin nanofibers). Then, a 2% by mass chitosan nanofiber aqueous dispersion prepared according to the description in WO2015/111686 was sterilized in an autoclave at 121° C. for 20 min. Thereafter, this aqueous dispersion was mixed and suspended in sterile distilled water (Otsuka Distilled Water, manufactured by Otsuka Pharmaceutical Factory Co., Ltd.) to 1% (w/v) to produce an aqueous dispersion containing sterile chitosan nanofibers. The produced chitosan nanofiber aqueous dispersion (8 mL) was added to the above-mentioned aqueous dispersion (2 mL) containing chitin nanofibers carrying vitronectin, and mixed by pipetting to obtain an aqueous dispersion (10 mL) containing 0.2% (w/v) chitin nanofibers carrying vitronectin and 0.8% (w/v) chitosan nanofibers. (In the present specification, the mixture of chitin nanofibers carrying vitronectin and chitosan nanofibers prepared here is sometimes simply referred to as “substrate of Preparation Example 1”, “Preparation Example 1”, or “substrate 1”).

### [Experimental Example 1] Stirring Culture 1 Using Substrate 1 (Comparison with Microcarrier)

Human umbilical cord-derived mesenchymal stem cells (manufactured by PromoCell, #C-12971) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 15 mL of a medium at a seeding concentration of 3×10⁴ cells/mL, and cultured for 10 days under various conditions in a CO₂ incubator (37° C., 5% CO₂). A 30 mL single-use reactor (manufactured by ABLE Corporation, #BWV-S03A) was used as a culture container, and an exclusive magnetic stirrer (manufactured by ABLE Corporation, #BWS-S03N0S-6) was used under stirring conditions. On days 4 and 7 of culture, the culture container was allowed to stand for 10 min, and half of the culture supernatant was replaced with the medium.

### (Culture Conditions)

In Comparative Example 1, Corning (registered trade mark) Low Concentration Synthemax (registered trade mark) II Microcarriers (manufactured by Corning, #3781) was weighed, immersed in disinfection ethanol (manufactured by Kaneichi Pharmaceutical. Co., Ltd., #4987556241025), replaced with mesenchymal stem cell growth medium 2, and culture was performed under stirring conditions using microcarrier equivalent to 300 mg. One cycle of stirring included 55 rpm for 1 min and 0 rpm for 59 min, 10 cycles of culture was performed under this condition, and then constant stirring was performed at 55 rpm. In Examples 1 and 2, a medium composition in which the substrate of Preparation Example 1 was added to mesenchymal stem cell growth medium 2 to a final concentration of 0.05% (w/v) was used. In Example 1, culture was performed under static conditions and culture was performed in Example 2 under stirring conditions similar to those in Comparative Example 1.

### (Calculation of Proliferation Rate)

On days 0, 1, 4, 7, and 10 of culture, 0.5 mL of the uniformly suspended culture solution was collected, and 0.5 mL of ATP reagent (CellTiter-Glo (registered trade mark) Luminescent Cell Viability Assay, manufactured by Promega) was added to each. The mixture was stirred with a vortex, allowed to stand at room temperature for 10 min, and dispensed into a white 96-well plate by 150 μL. The luminescence intensity (RLU value) was measured using Enspire (manufactured by Perkin Elmer), and the number of viable cells was determined by subtracting the luminescence value of the medium alone. The relative value when the RLU value on day 0 of culture was set to 1 was defined as the cell proliferation rate. The results are shown in Table 1.

TABLE 1

| | day 0 | day 1 | day 4 | day 7 | day 10 |
|---|---|---|---|---|---|
| Comparative | 1 | 1.4 | 12.7 | 14.9 | 12.3 |
| Example 1 | | | | | |
| Example 1 | 1 | 0.7 | 3.6 | 15.3 | 10.1 |
| Example 2 | 1 | 0.7 | 2.8 | 8.7 | 14.4 |

### (Medium Environment Measurement)

On days 0, 1, 4, 7, and 10 of culture, 0.5 mL of the uniformly suspended culture medium was collected into a 1.5 mL tube, and the culture supernatant was obtained by centrifugation (600×g, 3 min). Using FLEX2 (manufactured by nova biomedical), concentrations of glucose, lactic acid, and ammonia in the culture supernatant were measured. As the measurement value on day 0, the medium without cells before the start of culture was used for the measurement. The results are shown in Table 2.

TABLE 2

| | day 0 | day 1 | day 4 | day 7 | day 10 |
|---|---|---|---|---|---|
| glucose (mM) | | | | | |
| Comparative | 4.6 | 4.2 | 0.9 | 0.0 | 0.0 |
| Example 1 | | | | | |
| Example 1 | 4.6 | 4.3 | 3.2 | 0.0 | 0.0 |
| Example 2 | 4.6 | 4.2 | 3.2 | 0.0 | 0.0 |
| lactic acid (mM) | | | | | |
| Comparative | 1.1 | 3.7 | 8.1 | 8.1 | 7.5 |
| Example 1 | | | | | |
| Example 1 | 1.1 | 3.9 | 3.4 | 9.5 | 9.7 |
| Example 2 | 1.1 | 3.4 | 3.1 | 9.0 | 8.4 |
| ammonia (mM) | | | | | |
| Comparative | 0.11 | 0.15 | 1.00 | 2.46 | 2.43 |
| Example 1 | | | | | |
| Example 1 | 0.11 | 0.16 | 0.63 | 1.28 | 1.54 |
| Example 2 | 0.11 | 0.16 | 0.68 | 1.39 | 1.93 |

Regarding the proliferation rate, as shown in Table 1, the proliferation rate in Comparative Example 1 rapidly increased on day 4 and reached maximum on day 7. On the other hand, the proliferation rates in Examples 1 and 2 rapidly increased after day 4, reaching the maximum on day 7 in Example 1 and on day 10 in Example 2. The respective maximum values were approximately of the same level. From the above results, it was suggested that the maximum value of the proliferation rate was not different between the conditions in which the medium composition used in the Examples was left standing or constantly stirred and the conditions in Comparative Example 1.

Regarding the environment after culture, as shown in Table 2, the ammonia concentration in the culture medium in Examples 1 and 2 was always lower than that in Comparative Example 1. Furthermore, even though the glucose concentration in the medium became almost 0 on day 7 under any conditions, the proliferation rate in Example 2 increased over time until day 10. The above results suggest that culture with reduced cytotoxic ammonia is possible by using the medium composition used in the Examples, and that cells may proliferate even at low glucose concentrations.

### [Experimental Example 2] Stirring Culture 2 Using Substrate 1 (Stirring Conditions and CO₂ Concentration)

Human umbilical cord-derived mesenchymal stem cells (manufactured by PromoCell, #C-12971) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 30 mL of a medium composition in which the substrate of Preparation Example 1 was added to mesenchymal stem cell growth medium 2 to a final concentration 0.05% (w/v), such that the seeding concentration was 3×10⁴ cells/mL, and cultured for 7 days under various conditions in a CO₂ incubator (37° C., 5% or 10% CO₂). A 30 mL single-use reactor (manufactured by ABLE Corporation, #BWV-S03A) was used as a culture container, and an exclusive magnetic stirrer (manufactured by ABLE Corporation, #BWS-S03N0S-6) was used under stirring conditions. On day 4 of culture, the culture container was allowed to stand for 10 min, and half of the culture supernatant was replaced with the medium.

### (Culture Conditions)

Stirring and CO₂ conditions are shown below.
condition 1: standing still, 5% CO₂
condition 2: standing still, 10% CO₂
condition 3: constant stirring at 25 rpm, 5% CO₂

### (Calculation of Proliferation Rate)

On days 0, 1, 4, and 7 of culture, 0.5 mL of the uniformly suspended culture solution was collected, and 0.5 mL of ATP reagent (CellTiter-Glo (registered trade mark) Luminescent Cell Viability Assay, manufactured by Promega) was added to each. The mixture was stirred with a vortex, allowed to stand at room temperature for 10 min, and dispensed into a white 96-well plate by 150 μL. The luminescence intensity (RLU value) was measured using Enspire (manufactured by Perkin Elmer), and the number of viable cells was determined by subtracting the luminescence value of the medium alone. The relative value when the RLU value on day 0 of culture was set to 1 was defined as the cell proliferation rate. The results are shown in Table 3.

TABLE 3

| | day 0 | day 1 | day 4 | day 7 |
|---|---|---|---|---|
| condition 1 | 1.0 | 1.4 | 5.6 | 7.7 |
| condition 2 | 1.0 | 1.3 | 3.8 | 4.5 |
| condition 3 | 1.0 | 1.5 | 10.5 | 14.3 |

As shown in Table 3, the proliferation rate decreased under condition 2 compared to condition 1. Furthermore, the proliferation rate was improved under condition 3 compared to condition 1. The above results suggest that the stirring frequency during culture may affect the proliferation efficiency. Furthermore, it was suggested that the proliferation efficiency may be improved in constant stirring culture compared to static culture.

### [Experimental Example 3] Stirring Culture 3 Using Substrate 1 (Comparison with Static Culture)

Human umbilical cord-derived mesenchymal stem cells (manufactured by PromoCell, #C-12971) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 30 mL of a medium composition in which the substrate of Preparation Example 1 was added to mesenchymal stem cell growth medium 2 to a final concentration 0.05% (w/v), such that the seeding concentration was 3×10⁴ cells/mL, and cultured for 9 days in a CO₂ incubator (37° C., 5% CO₂). A 30 mL single-use reactor (manufactured by ABLE Corporation, #BWV-S03A) was used as a culture container, an exclusive magnetic stirrer (manufactured by ABLE Corporation, #BWS-S03N0S-6) was used under stirring conditions, and constant stirring was performed at 25 rpm. On days 4 and 7 of culture, the culture container was allowed to stand for 10 min, and half of the culture supernatant was replaced with the medium. As a comparison target, cells were seeded in a 24-well flat bottom adhesive surface microplate (#3526, manufactured by Corning Incorporated) at 5×10⁴ cells/well/1 mL and adhesion cultured.

### (Culture Conditions)

condition 4: standing still
condition 5: constant stirring

### (Calculation of Proliferation Rate)

On days 0, 1, 2, 3, 4, 7, 8, and 9 of culture, 0.5 mL of the uniformly suspended culture solution was collected, and 0.5 mL of ATP reagent (CellTiter-Glo (registered trade mark) Luminescent Cell Viability Assay, manufactured by Promega) was added to each. The mixture was stirred with a vortex, allowed to stand at room temperature for 10 min, and dispensed into a white 96-well plate by 150 μL. The luminescence intensity (RLU value) was measured using Enspire (manufactured by Perkin Elmer), and the number of viable cells was determined by subtracting the luminescence value of the medium alone. The relative value when the RLU value (ATP measurement, luminescence intensity) on day 0 of culture was set to 1 was defined as the cell proliferation rate. The results are shown in Table 4.

### (Gene Expression Analysis)

On days 0, 4, and 7, the cells were recovered, and RLT solution (700 μL, RNeasy mini kit (manufactured by QIAGEN, #74106)) was added to give an RNA extraction solution. To the RNA extraction solution was added 70% ethanol (700 μL), and the mixture was added to RNeasy spin column and centrifuged at 8000×g for 15 sec. Successively, 700 μL of RW1 solution was added to RNeasy spin column, and centrifuged at 8000×g for 15 sec. Successively, 500 μL of RPE solution was added, and centrifuged at 8000×g for 15 sec. Furthermore, 500 μL of RPE solution was added, and centrifuged at 8000×g for 2 min. RNase-free solution was added to RNAs remaining in the RNeasy spin column to elute them. Then, cDNAs were synthesized from the obtained RNAs by using PrimeScript RT reagent Kit (Perfect Real Time) (manufactured by Takara Bio Inc., #RR037A). Using the synthesized cDNAs, Premix EX Taq (Perfect Real Time) (manufactured by Takara Bio Inc., #RR039A), and Taq man Probe (manufactured by Applied Bio Systems), real-time PCR was performed. As the Taq man Probes (manufactured by Applied Bio Systems), Hs04260367_gH for OCT4, Hs01053049_s1 for SOX2, Hs04399610_g1 for NANOG, Hs00607978_s1 for CXCR4, and Hs99999905_m1 for GAPDH were used. As the instrument, real-time PCR7500 was used. In the analysis, relative values obtained by amending the values of each target gene with the values of GAPDH were calculated and compared using the cell on day 0 as 1. The results are shown in Table 5.

### (Cell Staining)

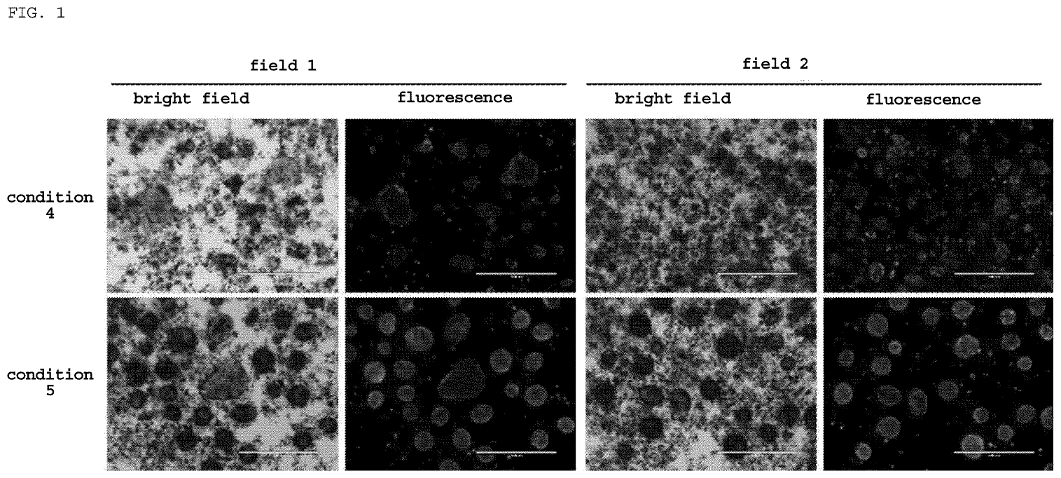

On day 7 of culture, 1 mL of the uniformly suspended culture medium was collected into a 1.5 mL tube, and after centrifugation (600×g, 3 min), the culture supernatant was removed. The cells were suspended in 1 mL of D-PBS(−) (manufactured by FUJIFILM Wako Pure Chemical Corporation, #045-29795), centrifuged (600×g, 3 min), and the culture supernatant was removed. A Calcein-AM (manufactured by DOJINDO LABORATORIES, #C326) solution (10 μL) dissolved in DMSO at a final concentration of 0.5 mg/mL was dissolved in 5 mL of D-PBS(−) (manufactured by FUJIFILM Wako Pure Chemical Corporation, #045-29795) and used as a staining solution. The cells were suspended in 1 mL of the staining solution, transferred to a 12-well plate (manufactured by Corning, #351143), and incubated in a CO₂ incubator (37° C., 5% CO₂) for 30 min. Thereafter, bright field images and viable cell-specific fluorescent staining images were obtained using EVOS (registered trade mark) FL Auto (manufactured by ThermoFisher). The results are shown in FIG. 1. The scale bar indicates 1000 μm.

### (Image Analysis)

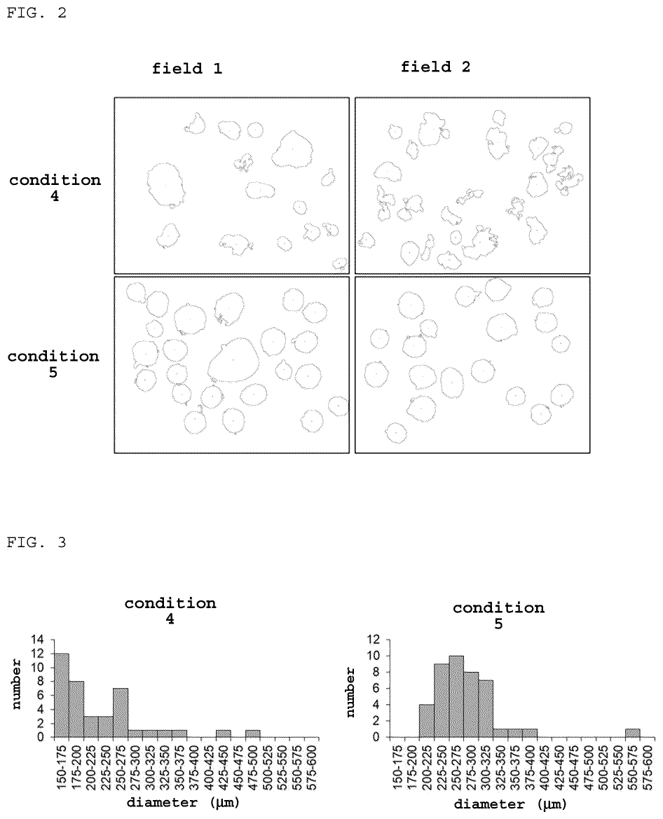

Using the fluorescent stained image obtained by cell staining, image analysis was performed using ImageJ (National Institutes of Health, 64-bit Java 1.8.0_172). As a pre-treatment for image analysis, the scale was standardized using a scale bar in the image, each image was converted to 32-bit, the brightness between each image was unified, Gaussian filter (Sigma value 2.00) was adapted, contour was extracted using Find Edges, Binary treatment and adaption of Close were performed, and images in which scale bars overlapped and images overlapped with image sides were removed. Using the image obtained by the pre-treatment, spheres with an area value of not less than 17671.46 (μm²) (average diameter of 150 μm or more) were extracted, and the number of spheres, area value (μm²), and circularity were obtained. Using the obtained area value, the sphere average diameter was calculated assuming that the sphere was a perfect circle, and the sphere average diameter, standard deviation, and size distribution data were obtained. The following formula was used to calculate the average sphere diameter. The sphere extraction image from which data was finally obtained is shown in FIG. 2, the number of spheres, average sphere diameter and standard deviation, and circularity and standard deviation are shown in Table 6, and the sphere size distribution is shown in FIG. 3. The X axis of FIG. 3 is, for example, the number of sphere population of not less than 150 μm and less than 175 μm in the case of 150-175.

$$sphere average diameter = 2 \times \sqrt{\frac{area value}{number π}}$$

TABLE 4

| | day 0 | day 1 | day 2 | day 3 | day 4 | day 7 | day 8 | day 9 |
|---|---|---|---|---|---|---|---|---|
| condition 4 | 1 | 1.3 | 2.1 | 2.9 | 4 | 9 | 9.5 | 7.9 |
| condition 5 | 1 | 1.3 | 2.2 | 3.7 | 6.9 | 12.2 | 16.6 | 15.5 |

As shown in Table 4, high proliferation rate was obtained in condition 5 as compared with condition 4.

TABLE 5

| | OCT4 | | Nanog | | CXCR4 | |
|---|---|---|---|---|---|---|
| | day 4 | day 7 | day 4 | day 7 | day 4 | day 7 |
| condition 4 | 9.0 | 14.7 | 14.7 | 20.6 | 14.1 | 21.3 |
| condition 5 | 12.0 | 27.0 | 15.8 | 34.3 | 16.1 | 32.1 |

As shown in Table 5, in conditions 4 and 5, relative gene expression levels of OCT4, Nanog, and CXCR4 increased over time as compared with the cells at the time of seeding. Furthermore, condition 5 showed higher values as compared with condition 4.

TABLE 6

| | number of | average | standard | | |
|---|---|---|---|---|---|
| | spheres | diameter | deviation | | standard |
| | (spheres) | (μm) | (μm) | circularity | deviation |
| condition 4 | 39 | 226 | 77.8 | 0.502 | 0.211 |
| condition 5 | 42 | 279.8 | 59.7 | 0.718 | 0.11 |

As shown in FIG. 3, under condition 4, the number of populations of not less than 150 μm and less than 175 μm is the largest, and a clear peak top cannot be seen. Under condition 5, the number of populations of not less than 250 μm and less than 275 μm is the largest, resulting in a bell-shaped distribution. As shown in Table 6, the standard deviation under condition 5 was smaller than that under condition 4, and the circularity was high. The above results suggest that more uniform spherical spheres can be obtained by stirring.

### [Experimental Example 4] Stirring Culture 4 Using Substrate 1 (Concentration of Substrate)

Human umbilical cord-derived mesenchymal stem cells (manufactured by PromoCell, #C-12971) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 30 mL of a medium composition in which the substrate of Preparation Example 1 was added to mesenchymal stem cell growth medium 2 to a final concentration 0.05% (w/v), 0.02% (w/v), or 0.01% (w/v), such that the seeding concentration was 3×10⁴ cells/mL, and stirring-cultured for 7 days in a CO₂ incubator (37° C., 5% CO₂). A 30 mL single-use reactor (manufactured by ABLE Corporation, #BWV-S03A) was used as a culture container, an exclusive magnetic stirrer (manufactured by ABLE Corporation, #BWS-S03N0S-6) was used, and constant stirring was performed at 25 rpm. On day 4 of culture, the culture container was allowed to stand for 10 min, and half of the culture supernatant was replaced with the medium.

### (Calculation of Proliferation Rate)

On days 0, 1, 2, 4, and 7 of culture, 0.5 mL of the uniformly suspended culture solution was collected, and 0.5 mL of ATP reagent (CellTiter-Glo (registered trade mark) Luminescent Cell Viability Assay, manufactured by Promega) was added to each. The mixture was stirred with a vortex, allowed to stand at room temperature for 10 min, and dispensed into a white 96-well plate by 150 μL. The luminescence intensity (RLU value) was measured using Enspire (manufactured by Perkin Elmer), and the number of viable cells was determined by subtracting the luminescence value of the medium alone. The relative value when the RLU value (ATP measurement, luminescence intensity) on day 0 of culture was set to 1 was defined as the cell proliferation rate. The results are shown in Table 7.

### (Cell Staining)

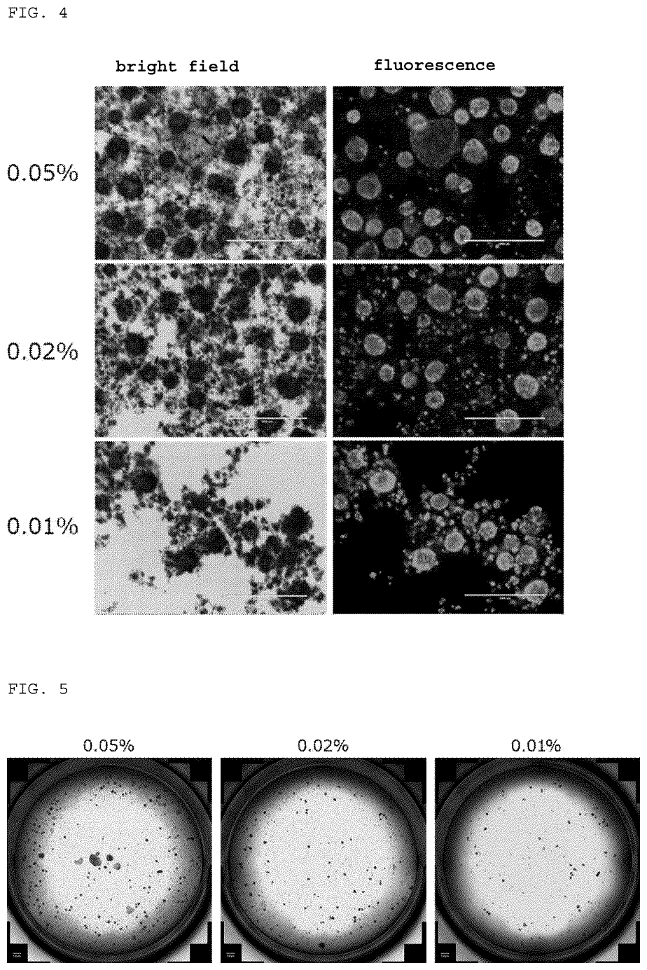

On day 7 of culture, 1 mL of the uniformly suspended culture medium was collected into a 1.5 mL tube, and after centrifugation (600×g, 3 min), the culture supernatant was removed. The cells were suspended in 1 mL of D-PBS(−) (manufactured by FUJIFILM Wako Pure Chemical Corporation, #045-29795), centrifuged (600×g, 3 min), and the culture supernatant was removed. A Calcein-AM (manufactured by DOJINDO LABORATORIES, #C326) solution (10 μL) dissolved in DMSO at a final concentration of 0.5 mg/mL was dissolved in 5 mL of D-PBS(−) (manufactured by FUJIFILM Wako Pure Chemical Corporation, #045-29795) and used as a staining solution. The cells were suspended in 1 mL of the staining solution, transferred to a 12-well plate (manufactured by Corning, #351143), and incubated in a CO₂ incubator (37° C., 5% CO₂) for 30 min. Thereafter, bright field images and viable cell-specific fluorescent staining images were obtained using EVOS (registered trade mark) FL Auto (manufactured by ThermoFisher). The representative image is shown in FIG. 4. The scale bar indicates 1000 μm.

### (Image Acquisition)

On day 7 of culture, uniformly suspended culture medium (0.5 mL) was collected in a 12-well plate, and the entire wells were photographed using Cell3iMagerduos (manufactured by SCREEN Holdings Co., Ltd.). The obtained images are shown in FIG. 5.

### (Image Analysis)

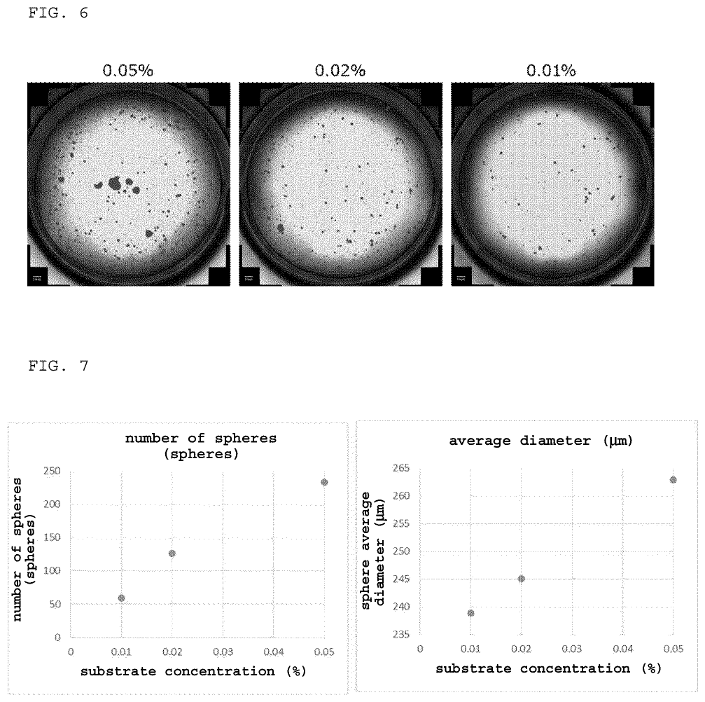

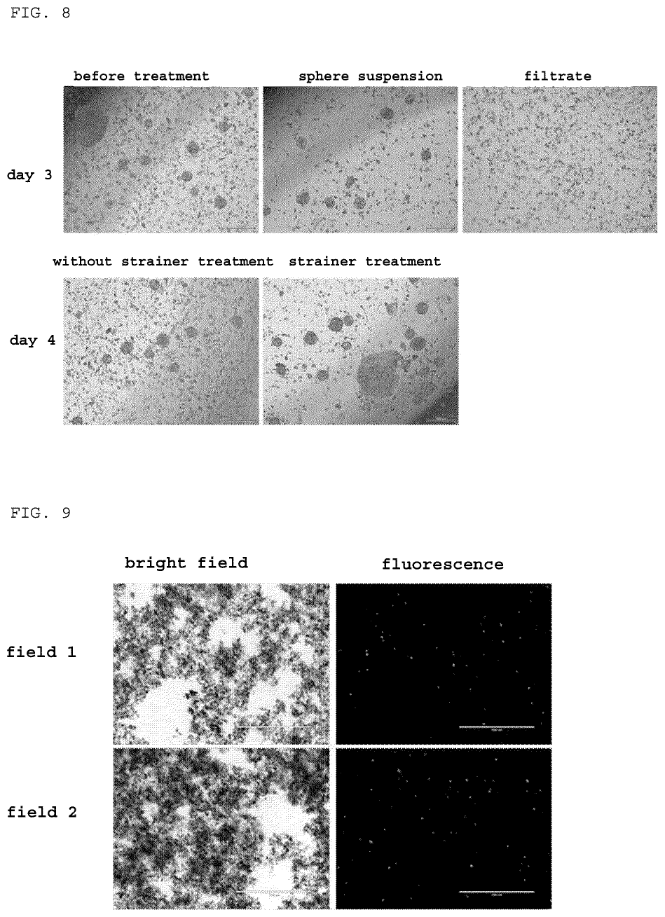

Using the obtained images of the entire wells, image analysis was performed using ImageJ (National Institutes of Health, 64-bit Java 1.8.0_172). After standardizing the scale using the scale bar in the image, the outline of the sphere was extracted using the Polygon selections tool, and the number of spheres and area value (μm²) were obtained. Using the acquired area values, the sphere average diameter was calculated assuming that the sphere was a perfect circle, and the sphere average diameter was obtained. The following formula was used to calculate the average sphere diameter. The sphere extracted image from which the data was finally obtained is shown in FIG. 6, and the number of spheres and sphere average diameter are shown in FIG. 7. Air bubbles were excluded from contour extraction.

$$sphere average diameter = 2 \times \sqrt{\frac{area value}{number π}}$$

TABLE 7

| | day 0 | day 1 | day 2 | day 4 | day 7 |
|---|---|---|---|---|---|
| 0.05% | 1.0 | 1.6 | 2.3 | 5.5 | 15.1 |
| 0.02% | 1.0 | 1.6 | 2.7 | 5.7 | 8.4 |
| 0.01% | 1.0 | 1.7 | 2.4 | 4.4 | 6.4 |

As shown in Table 7 and FIG. 7, it was shown that the present invention can be practiced even when the concentration of substrate 1 is changed.

### [Experimental Example 5] Separation of Spheres

Human umbilical cord-derived mesenchymal stem cells (manufactured by PromoCell, #C-12971) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 30 mL of a medium composition in which the substrate of Preparation Example 1 was added to mesenchymal stem cell growth medium 2 to a final concentration 0.05% (w/v), such that the seeding concentration was 3×10⁴ cells/mL, and cultured for 4 days under stirring conditions of 25 rpm in a CO₂ incubator (37° C., 5% CO₂). A 30 mL single-use reactor (manufactured by ABLE Corporation, #BWV-S03A) as a culture container and an exclusive magnetic stirrer (manufactured by ABLE Corporation, #BWS-S03N0S-6) were used.

### (Separation of Spheres)

On day 3 of culture, the entire amount of the culture medium was passed through a cell strainer with a pore size of 60 μm (manufactured by pluriSelect, #43-50060-03), the mesh was turned upside down and washed with an equal volume of mesenchymal stem cell growth medium 2 to collect the spheres trapped on the mesh to give a sphere suspension. The solution that passed through the mesh was used as a filtrate.

### (Microscopic Observation)

0.5 mL of the uniformly suspended culture medium before passing through the cell strainer, the sphere suspension, and the filtrate were transferred into a 12-well plate (manufactured by Corning, #351143), and observed under an inverted microscope (manufactured by Olympus Corporation, #IX73). In addition, a culture medium obtained by further culturing the sphere suspension for one more day and a suspension obtained by culturing the suspension for four days without a cell strainer treatment were similarly observed. The acquired image is shown in FIG. 8. The scale bar indicates 500 μm.

### (Staining)

1 mL of the obtained filtrate was collected into a 1.5 mL tube, and after centrifugation (600×g, 3 min), the supernatant was removed. The pellets were suspended in 1 mL of D-PBS(−) (manufactured by FUJIFILM Wako Pure Chemical Corporation, #045-29795), centrifuged (600×g, 3 min), and the culture supernatant was removed. A Calcein-AM (manufactured by DOJINDO LABORATORIES, #C326) solution (10 μL) dissolved in DMSO at a final concentration of 0.5 mg/mL was dissolved in 5 mL of D-PBS(−) (manufactured by FUJIFILM Wako Pure Chemical Corporation, #045-29795) and used as a staining solution. The pellets were suspended in 1 mL of the staining solution, transferred to a 12-well plate (manufactured by Corning, #351143), and incubated in a CO₂ incubator (37° C., 5% CO₂) for 30 min. Thereafter, bright field images and viable cell-specific fluorescent staining images were obtained using EVOS (registered trade mark) FL Auto (manufactured by ThermoFisher). The acquired image is shown in FIG. 9. The scale bar indicates 1000 μm.

As shown in FIG. 8 and FIG. 9, it was clarified that by using a cell strainer, the spheres can be separated from the excess substrates, and the culture can be continued. The above results suggest that it is possible to separate only spheres from the substrates, single cells, and dead cells by using a mesh.

### [Experimental Example 6] Scale-Up Culture

A medium containing mesenchymal stem cell growth medium 2 (manufactured by PromoCell, #C-28009) and Penicillin-Streptomycin Solution (×100) (manufactured by FUJIFILM Wako Pure Chemical Corporation, #168-23191) was added to UniVessel (registered trade mark) Glass1 L (manufactured by Sartorius) sterilized by autoclaving (121° C., 20 min), connected to BIOSTAT (registered trade mark) B-DCU (manufactured by Sartorius), and the medium was then conditioned for 30 min under the conditions of compression air 130 ccm and CO₂ 10 ccm, 37° C., 120 rpm. Human umbilical cord-derived mesenchymal stem cells (manufactured by PromoCell, #C-12971) were adhesion cultured for 3 days on a 10 cm dish (manufactured by Corning, #430167) using mesenchymal stem cell growth medium 2. Thereafter, the cells were detached using DetachKit (manufactured by PromoCell, #C-41210), and added at 3×10⁴ cells/mL seeding concentration to a medium that had been conditioned in advance so that the final concentration of the substrate of Preparation Example 1 was 0.05% (w/v). The total amount of the medium was 424 mL. Culture was performed with stirring for 11 days under the conditions of compression air 130 ccm and CO₂ 8 or 10 ccm, 37° C., 45 or 60 rpm. On days 4 and 7 of culture, half of the cell suspension was collected in the reactor, centrifuged (300×g, 3 min, Deccel mode), the supernatant was removed, the cells were suspended in a new medium and returned to the reactor, whereby the medium was replaced.

### (Calculation of Proliferation Rate)

On days 0, 1, 4, 7, and 11 of culture, 0.5 mL of the uniformly suspended culture solution was collected, and 0.5 mL of ATP reagent (CellTiter-Glo (registered trade mark) Luminescent Cell Viability Assay, manufactured by Promega) was added to each. The mixture was stirred with a vortex, allowed to stand at room temperature for 10 min, and dispensed into a white 96-well plate by 150 μL. The luminescence intensity (RLU value) was measured by Enspire (manufactured by Perkin Elmer) and the number of viable cells was measured by subtracting the luminescence value of the medium alone. The relative value when the RLU value (ATP measurement, luminescence intensity) on day 0 of culture was set to 1 was defined as the cell proliferation rate. The results are shown in Table 8

### (Separation of Spheres)

On day 11 of culture, 50 mL of the culture medium was passed through a cell strainer with a pore size of 100 n (manufactured by pluriSelect, #43-50100-03) or 200 m (manufactured by pluriSelect, #43-50200-03), the spheres trapped on the mesh were washed by adding D-PBS(−) (manufactured by FUJIFILM Wako Pure Chemical Corporation, #045-29795), the mesh was turned upside down and washed with an appropriate amount of D-PBS(−) to collect the spheres trapped on the mesh to give a sphere suspension. The solution that passed through the mesh was used as a filtrate.

### (Microscopic Observation)

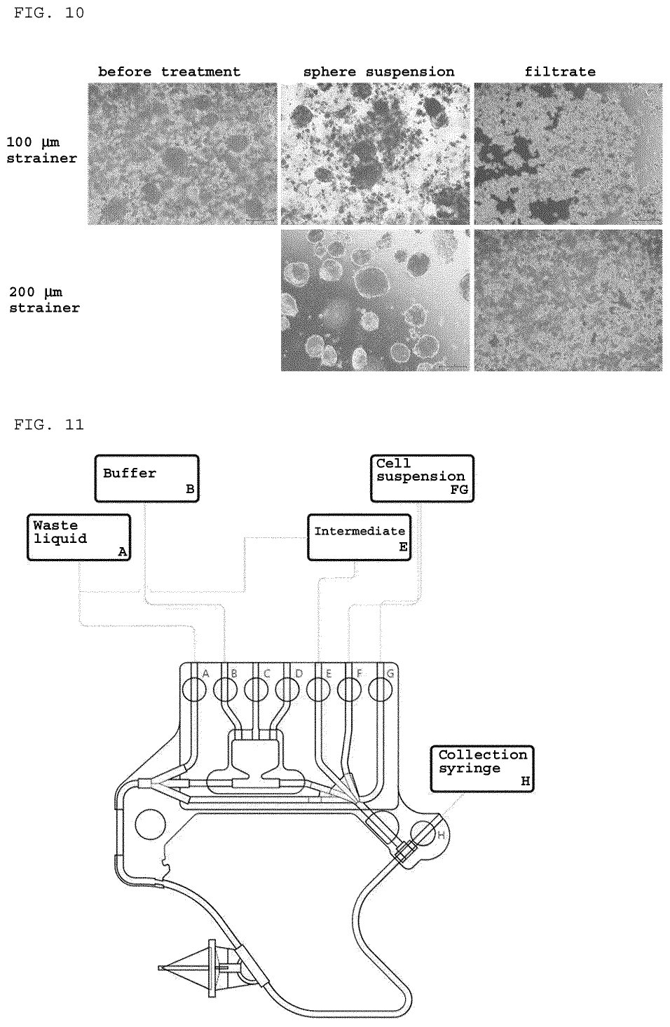

1 mL of the uniformly suspended culture medium before passing through the cell strainer, the sphere suspension, and the filtrate were transferred into a 12-well plate (manufactured by Corning, #351143), and observed under an inverted microscope (manufactured by Olympus Corporation, #IX73). The acquired image is shown in FIG. 10. The scale bar indicates 500 μm.

TABLE 8

| | day 0 | day 1 | day 4 | day 7 | day 11 |
|---|---|---|---|---|---|
| proliferation rate | 1.0 | 1.5 | 2.8 | 6.4 | 12.2 |

As shown in Table 8, it was clarified that cells proliferate over time even under scale-up conditions. The blades of the Sartorius bioreactor used in this Example have a screw-type shape, and the blades manufactured by ABLE Corporation used in the previous Examples have a delta-type shape. This suggests that the scale-up culture may be possible regardless of the blade shape.

As shown in FIG. 10, it was clarified that the spheres and excess substrates can be separated using a cell strainer. When a cell strainer with a pore size of 100 μm was used, substrates smaller than the mesh pore size could not be cleaned due to clogging. Using a cell strainer with a pore size of 200 μm, clogging could be eliminated and contamination of small substrates into the sphere suspension after cleaning was less. From the above results, it was suggested that improvements in cleaning efficiency and increase in the amount of liquid that can be processed can be expected by selecting a cell strainer with an appropriate pore size for collecting and cleaning spheres.

### [Experimental Example 7] Dispersing Spheres into Single Cells

Human umbilical cord-derived mesenchymal stem cells (manufactured by PromoCell, #C-12971) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 100 mL of a medium composition in which the substrate of Preparation Example 1 was added to mesenchymal stem cell growth medium 2 to a final concentration 0.05% (w/v), such that the seeding concentration was 3×10⁴ cells/mL, and cultured for 10 days under various conditions in a CO₂ incubator (37° C., 5% CO₂) A 100 mL single-use reactor (manufactured by ABLE Corporation, #BWV-S10A) was used as a culture container, an exclusive magnetic stirrer (manufactured by ABLE Corporation, #BWS-S03N0S-6) was used, and constant stirring was performed at 25 rpm. On day 3 of culture, the entire amount of the culture medium was passed through a cell strainer with a pore size of 60 μm (manufactured by pluriSelect, #43-50060-03), the mesh was turned upside down and washed with an equal volume of mesenchymal stem cell growth medium 2 to collect the spheres trapped on the mesh to continue culture. On day 7 of culture, the culture container was allowed to stand for 10 min, and half of the culture supernatant was replaced with the medium.

### (Enzyme Treatment)

5 mL of D-MEM medium (manufactured by FUJIFILM Wako Pure Chemical Corporation, #043-30085) was added to each of two Dri Tumor & Tissue Dissociation Reagent (TTDR) (manufactured by BD, #661563) vials, and dissolved at room temperature for 15 min with appropriate mixing. 2 mL of TrypLE (registered trade mark) Select Enzyme (10×), no phenol red (manufactured by Thermo Fisher, #A1217701) was added to a total 10 mL of TTDR solution to give an enzyme solution. 60 mL of the uniformly suspended culture medium was collected into two 50 mL tubes, and after centrifugation (300×g, 3 min), the culture supernatant was removed. The pellet was suspended in 50 mL of D-MEM medium, combined into one tube, centrifuged (300×g, 3 min), and the supernatant was removed. The pellet was suspended in an appropriate amount of D-MEM medium, combined with the enzyme solution to adjust the volume to 20 mL, transferred to a 100 mL single-use reactor, and stirred in a CO₂ incubator (37° C., 5% CO₂) for 30 min at 25 rpm using an exclusive magnetic stirrer. Thereafter, 30 mL of D-MEM medium containing 2% FBS was added to the reactor and the cell suspension was transferred to a process bag.

### (Separation)

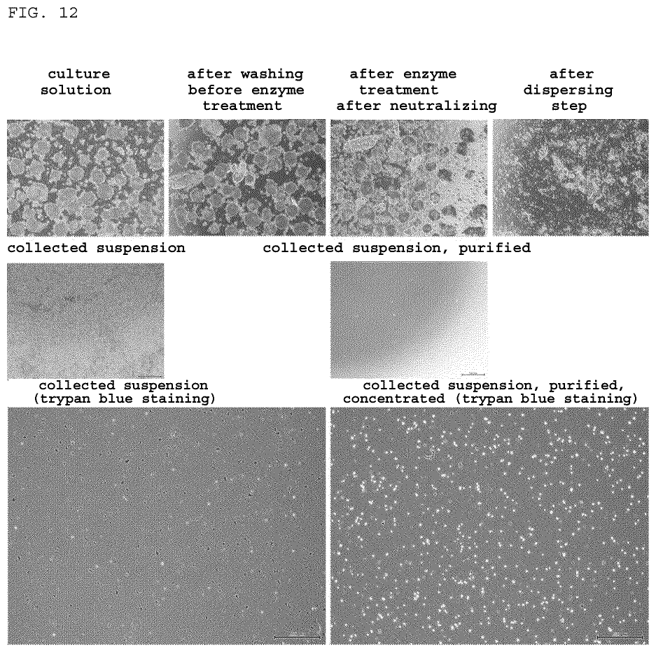

A drainage bag, a buffer bag containing D-MEM medium containing 2% FBS, a temporary storage bag, a bag containing cell suspension, and a syringe for cell collection were connected to Rotea Single-Use Kit (manufactured by Thermo Fisher, #A45130). The flow path was branched between the drainage bag and the kit, and a channel connecting to the storage bag was added. A schematic diagram of the connection mode is shown in FIG. 11. Thereafter, it was installed in Rotea (manufactured by Thermo Fisher) and the spheres were dispersed and the cells were separated. The kit channel was used in each step after air bubbles in the channel were removed by priming. In the sphere dispersion step, spheres were dispersed into single cells by repeating bet formation and disintegration of the formed bet 10 times by changing the centrifugal strength to 100×g or 2000×g at 110 mL/min. The separation step was performed at 110 mL/min and 800×g in the first stage, and at 2500×g at 50 mL/min in the second stage. In each washing step, 50 mL of buffer was used to wash the formed bet. In the cell collection step, a cell fraction, which is a bet formed by sending 20 mL of buffer, was collected into a syringe. The flow of liquid and the channels used in each step are shown in Table 9.

### (Purification)

A density gradient solution was prepared by adding and mixing 4.5 mL of Percoll (manufactured by Cytiva, #17089101) and 0.5 mL of 10×D-PBS(−) (manufactured by FUJIFILM Wako Pure Chemical Corporation, #048-29805) and 4 mL of MEM medium containing 2% FBS to four 15 mL tubes, and a density gradient was formed by centrifugation (400×g, 10 min, Slow Deccel mode). Then, 5 mL of cell fraction was slowly dispensed into each 15 mL tube, and after centrifugation (400×g, 10 min, Slow Deccel mode), the cell fractions found near the density gradient interface were collected into a 15 mL tube, and the cells were concentrated by centrifugation (400×g, 3 min).

### (Microscopic Observation)

0.5 mL or 1 mL of the uniformly suspended culture medium at each stage was transferred into a 12-well plate (manufactured by Corning, #351143), and observed under an inverted microscope (manufactured by Olympus Corporation, #IX73). Immediately after collection and after purification and concentration of the cell fractions, dead cells and the substrates were specifically stained with trypan blue solution (manufactured by FUJIFILM Wako Pure Chemical Corporation, #207-17081), then added to counting slides (manufactured by Bio-Rad, #1450011) and similarly observed. The acquired image is shown in FIG. 12. The scale bar indicates 500 μm.

TABLE 9

| | | container | | channel | |
|---|---|---|---|---|---|
| procedure | treatment | feed source | receiver | start | return |
| 1 | sphere | Cell | Cell | F | G |
| | dispersion | suspension | suspension | | |
| 2 | 1st stage | Cell | Intermidiate | F | A |
| | separation | suspension | | | |
| 3 | wash | Buffer | Drainage | B | A |
| 4 | bet | Buffer | Cell | B | F |
| | recovery | | suspension | | |
| 5 | 2nd stage | Intermidiate | Drainage | E | A |
| | separation | | | | |
| 6 | wash | Buffer | Drainage | B | A |
| 7 | cell | Buffer | Syringe | B | H |
| | recovery | | | | |

As shown in FIG. 12, it was clarified that the spheres were dispersed into single cells after the dispersion step, and that the single cells with high purity can be recovered through purification. The above results suggest that the formed spheres can be dispersed into single cells and can be recovered as single cells.

### [Experimental Example 8] Examination of the Concentration of Substrate 1 and Cell Recovery Efficiency—1

The substrate of Preparation Example 1 was added to mesenchymal stem cell growth medium 2 (manufactured by PromoCell, #C-28009) to a final concentration of 0.05 or 0.01% (w/v) to prepare medium compositions.

Successively, the cultured human adipose tissue-derived mesenchymal stem cells (manufactured by CellSource Co., Ltd., #0111201) were respectively suspended in each of the above-mentioned medium compositions at 1.5×10⁴ cells/mL, and seeded in a 6-well flat-bottomed ultra low attachment surface microplate (manufactured by Corning Incorporated, #3471) at 10 mL/well. The cells were cultured in a CO₂ incubator (37° C., 5% CO₂) in a static state. On day 3, about 5 mL of the medium supernatant in the well was removed, a fresh mesenchymal stem cell proliferation medium (5 mL) was added to each well, suspended by pipetting, thereby exchanging half volume of the medium, and culture was continued until day 7 after seeding. On day 7, half of the culture supernatant was removed, and the remainder was collected in a 50 mL conical tube. It was left standing for 15 min to allow the spheroids to naturally settle, and the remaining culture supernatant was removed. HBSS(−) (manufactured by Thermo Fisher, #14175095) (40 mL) was added thereto, and the mixture was allowed to stand for 15 min again to allow the spheroid composition to settle naturally, and then the supernatant was removed and washed. Then, liberase (manufactured by Merck, #5401119001) (5 mg) dissolved in HBSS (−) (2 mL) (100 μL), TrypLE Select Enzyme (10×), no phenol red (manufactured by Thermo Fisher, #A1217701) (0.75 mL) were added and the mixture was incubated in a CO₂ incubator (37° C., 5% CO₂) for 1 hr to detach the cells from the substrates. The obtained cell/substrate suspension (A) was filtered using a cell strainer with a mesh diameter of 100 μm (manufactured by pluriSelect, #43-50100-51), and the filtrate was washed with HBSS(−) (3 mL) to obtain filtrate (B) containing cells separated from the substrates. In addition, the filtrate was backwashed with HBSS(−) (10 mL) to obtain suspension (C) of the filtrate.

An ATP reagent (500 μL, CellTiter-Glo™ Luminescent Cell Viability Assay, manufactured by Promega) was added to 500 μL of the above-mentioned A, B, and C respectively, and they were suspended and stood for about 10 min at room temperature, and dispensed into 3 wells of a white 96 well plate by 300 μL/well. The luminescence intensity (RLU value) was measured by a plate reader (manufactured by Tecan, infiniteM200PRO), and the luminescence value of the medium alone was subtracted to calculate viable cell amount (mean of the 3 points). Furthermore, the final ATP value was calculated by dividing by the volume of each suspension. The converted RLU value (ATP measurement, luminescence intensity) of each suspension is shown in Table 10.

TABLE 10

| | 0.05% | 0.01% |
|---|---|---|
| A) after detachment | 1648419 | 866728 |
| B) crude recovery | 1303888 | 768530.1 |
| C) residue | 347153.6 | 56077.41 |
| B/A × 100 (%) | 79.1 | 88.7 |
| C/A × 100 (%) | 21.1 | 6.5 |

As is clear from Table 10, it was confirmed that the amount of cells remaining on the substrate side was smaller when the concentration of substrate 1 was 0.01% than when it was 0.05%. Therefrom it was found that the efficiency of recovering cells (single cells) was improved by reducing the amount of substrate 1.

### [Experimental Example 9] Examination of the Concentration of Substrate 1 and Cell Recovery Efficiency—2

The substrate of Preparation Example 1 was added to mesenchymal stem cell growth medium 2 (manufactured by PromoCell, #C-28009) to a final concentration of 0.100% (w/v), 0.050% (w/v), 0.020% (w/v), 0.010% (w/v), 0.005% (w/v), or 0.003% (w/v) to prepare medium compositions.

Successively, the cultured human adipose tissue-derived mesenchymal stem cells (manufactured by CellSource Co., Ltd., #0111201) were respectively suspended in each of the above-mentioned medium compositions at 1.5×10⁴ cells/mL, and seeded in a 6-well flat-bottomed ultra low attachment surface microplate (manufactured by Corning Incorporated, #3471) at 10 mL/well. The cells were cultured in a CO₂ incubator (37° C., 5% CO₂) in a static state. On day 3, about 5 mL of the medium supernatant in the well was removed, a fresh mesenchymal stem cell proliferation medium (5 mL) was added to each well, suspended by pipetting, thereby exchanging half volume of the medium, and culture was continued until day 7 after seeding. On day 7, the entire amount (10 mL) of each well was collected in a 15 mL conical tube. It was left standing for 15 min to allow the spheroids to naturally settle, and the remaining culture supernatant was removed. HBSS(−) (manufactured by Thermo Fisher, #14175095) (8 mL) was added thereto, and the mixture was allowed to stand for 15 min again to allow the spheroid composition to settle naturally, and then the supernatant was removed and washed. Then, liberase (manufactured by Merck, #5401119001) (5 mg) dissolved in HBSS (−) (2 mL) (80 μL), TrypLE Select Enzyme (10×), no phenol red (manufactured by Thermo Fisher, #A1217701) (300 μL) were added and the mixture was incubated in a CO₂ incubator (37° C., 5% CO₂) for 1 hr to detach the cells from the substrates. 5 mL from 5.5 mL of the obtained cell/substrate suspension (A) diluted with HBSS (−) (4.1 mL) was filtered using a cell strainer with a mesh diameter of 70 μm (manufactured by pluriselect, #43-50070-51) to obtain filtrate (B) (5 mL) containing cells separated from the substrates. In addition, the filtrate was backwashed with HBSS(−) (5 mL) to obtain suspension (C) (5 mL) of the filtrate.

An ATP reagent (500 μL, CellTiter-Glo™ Luminescent Cell Viability Assay, manufactured by Promega) was added to 500 μL of the above-mentioned A, B, and C respectively, and they were suspended and stood for about 10 min at room temperature, and dispensed into 3 wells of a white 96 well plate by 300 μL/well. The luminescence intensity (RLU value) was measured by a plate reader (manufactured by Tecan, infiniteM200PRO), and the luminescence value of the medium alone was subtracted to calculate viable cell amount (mean of the 3 points). The converted RLU value (ATP measurement, luminescence intensity) of each suspension and the proportion before and after passing through the cell strainer are shown in Table 11.

TABLE 11

| | 0.100% | 0.050% | 0.020% | 0.010% | 0.005% | 0.003% |
|---|---|---|---|---|---|---|
| A) after detachment | 1885590 | 1905625 | 1213939 | 964686 | 961910.7 | 1182036 |
| B) recovery | 665443 | 933382.3 | 846151 | 802487.3 | 905131 | 1130898 |
| C) residue | 1543612 | 1396844 | 441238 | 95386.67 | 91855.67 | 84227.67 |
| recovery rate | 29.4 | 40.8 | 63.4 | 83.2 | 94.1 | 95.7 |
| B/A × 100(%) | | | | | | |
| residual ratio | 68.2 | 61.1 | 33 | 9.9 | 9.5 | 7.1 |
| C/A × 100(%) | | | | | | |

As is clear from Table 11, it was confirmed that the recovery rate improved as the concentration of the substrate was reduced. In addition, even though 0.010%, 0.005%, and 0.003% have about half the ATP value compared to 0.100% and 0.050%, the ATP value at the time of collection was equal to or more than that of 0.100% and 0.050%. Therefrom it was found that the efficiency of cell (single cell) recovery is improved by reducing the amount of substrate.

### [Experimental Example 10] Culture Under Shaking Conditions (Comparison with Static Culture)

The substrate of Preparation Example 1 was added to mesenchymal stem cell growth medium 2 (manufactured by PromoCell, #C-28009) to a final concentration of 0.100% (w/v) or 0.020% (w/v) to prepare medium compositions.

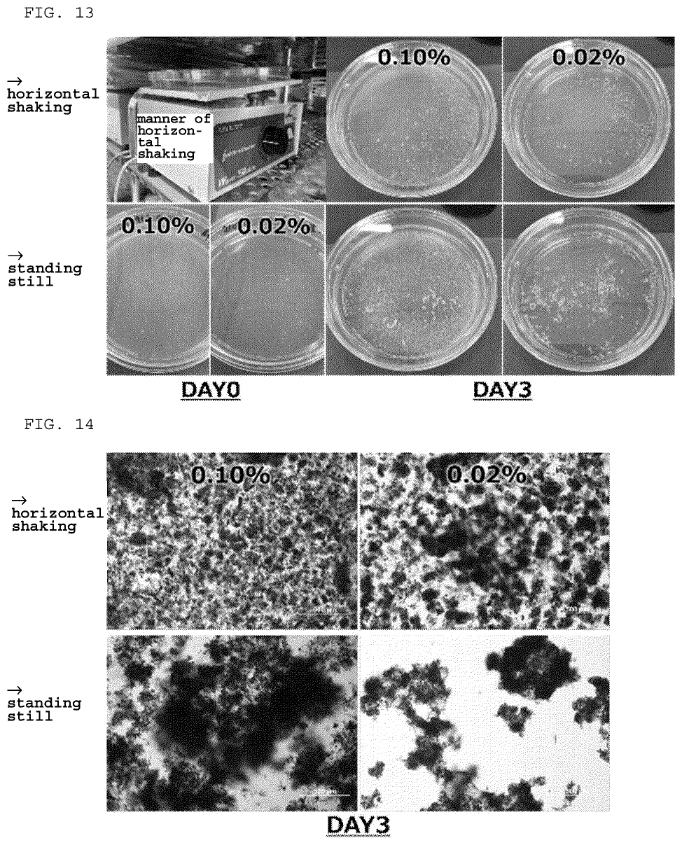

Successively, human adipose tissue-derived mesenchymal stem cells (manufactured by CellSource Co., Ltd., #0111201) were respectively suspended in each of the above-mentioned medium compositions at 3×10\hat{ }4 cells/mL, and seeded in a 100 mm flat bottom ultra-low adhesion surface dish (manufactured by Corning Incorporated, #3262) at 30 mL/dish. The cells were cultured in a CO₂ incubator (37° C., 5% CO₂) in a static state or in a horizontal shaking state by placing on an in vitro shaker (manufactured by TAITEC CORPORATION, wave-SI slim, SPEED: 15 setting). Photographs of the appearance on day 0 and day 3 of culture are shown in FIG. 13, and microscopic images are shown in FIG. 14. From the observation results, it was confirmed that more uniform spheroids were formed in shaking culture than in static culture.

Culture was continued until day 8 after seeding, and on day 8, the entire amount (30 mL) from each dish was collected in a 50 mL conical tube. It was left standing for 15 min to allow the spheroids to naturally settle, and the remaining culture supernatant was removed. HBSS(−) (manufactured by Thermo Fisher, #14175095) (20 mL) was added thereto, and the mixture was allowed to stand for 15 min again to allow the spheroid composition to settle naturally, and then the supernatant excluding 5 mL on the bottom was removed and washed. Then, liberase (manufactured by Merck, #5401119001) (5 mg) dissolved in HBSS (−) (2 mL) (400 μL), TrypLE Select Enzyme (10×), no phenol red (manufactured by Thermo Fisher, #A1217701) (1.4 mL) were added and the mixture was incubated in a CO₂ incubator (37° C., 5% CO₂) for 1 hr to detach the cells from the substrates. 15 mL of the obtained cell/substrate suspension (A) diluted with HBSS(−) (8.2 mL) was filtered using a cell strainer with a mesh diameter of 70 μm (manufactured by pluriSelect, #43-50070-51) to obtain filtrate (B) (15 mL) containing cells separated from the substrates. In addition, the filtrate was backwashed with HBSS(−) (15 mL) to obtain suspension (C) (15 mL) of the filtrate.

The cell concentration of the above-mentioned suspension C was measured using a cell counter (manufactured by BIO-RAD, TC-20), and the number of each recovered cell was calculated. The number of cells recovered at each concentration and the presence or absence of shaking is shown in Table 12. It was confirmed at any concentration that the cell yield was higher under horizontal shaking conditions than under static conditions. Therefrom it is suggested that not only stirring but also shaking enhances the formation of spheroids containing substrate, and that forming spheroids improves the subsequent so recovery of single cells.

TABLE 12

| substrate | 0.100% | | 0.020% | |
|---|---|---|---|---|
| concentration (%) | | | | |
| presence or absence | absence | presence | absence | presence |
| of shaking | | | | |
| number of recovered cells | 2.76 | 3.25 | 3.15 | 5.11 |
| (×10\hat{ }6 cells) | | | | |

### [Experimental Example 11] Comparison of Dispersion of Spheres into Single Cells

Human umbilical cord-derived mesenchymal stem cells (manufactured by PromoCell, #C-12971) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 30 mL of a medium composition in which the substrate of Preparation Example 1 was added to mesenchymal stem cell growth medium 2 to a final concentration 0.05% (w/v), such that the seeding concentration was 3×10⁴ cells/mL, and cultured for 11 days under stirring conditions of 25 rpm in a CO₂ incubator (37° C., 5% CO₂). A 30 mL single-use reactor (manufactured by ABLE Corporation, #BWV-S03A) as a culture container and an exclusive magnetic stirrer (manufactured by ABLE Corporation, #BWS-S03N0S-6) were used. On days 4 and 7 of culture, the culture container was allowed to stand for 10 min, and half of the culture supernatant was replaced with the medium. In Comparative Example, cells were seeded at 4 or 8×10³ cells/well/200 μL on a PrimeSurface (registered trade mark) plate 96U (manufactured by SUMITOMO BAKELITE CO., LTD., #MS-9096U), and static culture was performed for 4 days in a CO₂ incubator (37° C., 5% CO₂). The medium used in Comparative Example was mesenchymal stem cell growth medium 2 to which the substrate of Preparation Example 1 was not added.

### (Pre-Treatment)

The uniformly suspended culture solution was passed through a cell strainer with a pore size of 400 μm (manufactured by pluriSelect, #43-50400-03), the spheres trapped on the mesh were washed by adding D-PBS(−) (manufactured by FUJIFILM Wako Pure Chemical Corporation, #045-29795), the mesh was turned upside down and washed with an appropriate amount of D-PBS(−) to collect the spheres trapped on the mesh, and 0.9 mL of the cell suspension was transferred to a 12-well plate (manufactured by Corning, #351143). This condition was set as Example 3. In Comparative Example, the spheres were collected from the plate into a 15 mL tube and allowed to settle naturally. The supernatant was removed, D-PBS(−) was added, the spheres were allowed to settle naturally again, and the supernatant was removed to wash the spheres. The spheres were suspended in 0.9 mL of D-PBS(−) and transferred to a 12-well plate. Condition using spheres obtained by seeding at 4×10³ cells/well was set as Comparative Example 2, and condition using spheres obtained by seeding at 8×10³ cells/well was set as Comparative Example 3.

### (Enzyme Treatment, Cell Staining)

10 μL of Calcein-AM (manufactured by DOJINDO LABORATORIES, #C326) solution dissolved in DMSO at a final concentration of 0.5 mg/mL was added to 0.5 mL of D-PBS(−) or TrypLE (registered trade mark) Select Enzyme (10×), no phenol red (manufactured by Thermo Fisher, #A1217701), 0.1 mL was added to a 12-well plate, and the plate was incubated in a CO₂ incubator (37° C., 5% CO₂) for 30 min. Thereafter, under conditions in which TrypLE was added, pipetting was performed 20 times using Eppendorf Research (registered trade mark) plus 100-1000 μL (manufactured by Eppendorf, #3120000062) with a discharge volume set to 0.5 mL.

### (Image Acquisition)

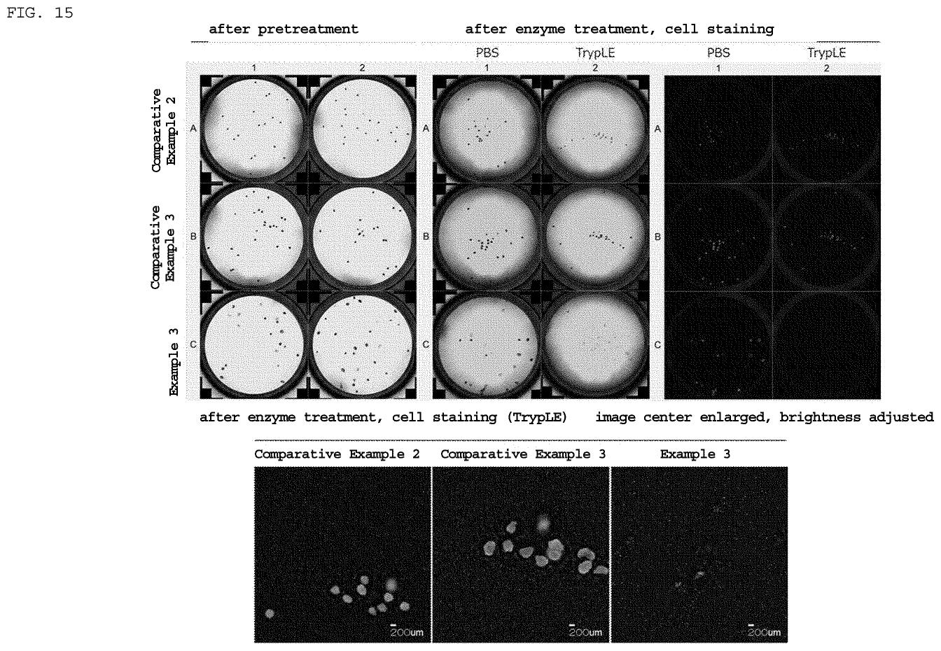

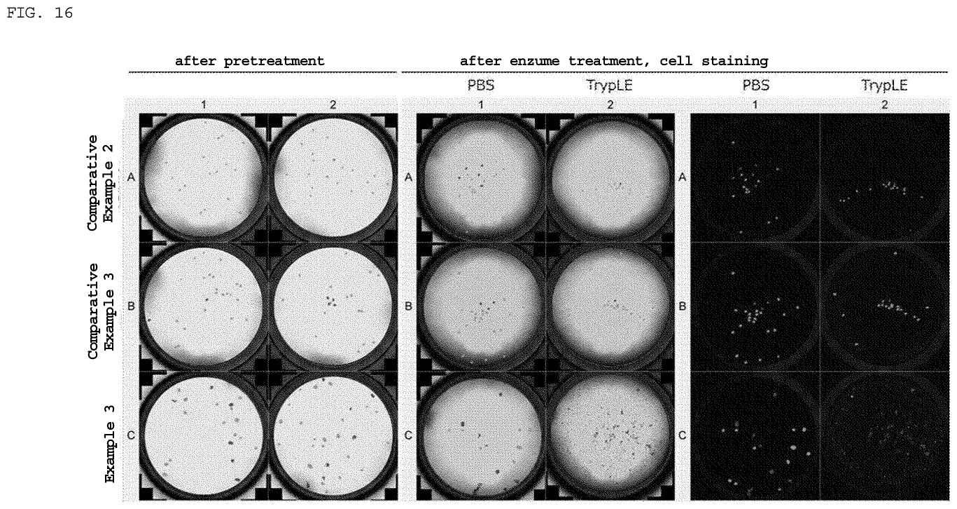

After a pretreatment and an enzyme treatment, the entire well of the 12-well plate was photographed using Cell3iMagerduos (manufactured by SCREEN Holdings Co., Ltd.) to obtain a bright field image and a viable cell-specific fluorescent staining image. The images are shown in FIG. 15. Using the acquired images, the average sphere diameter was calculated using Cell3iMagerduos built-in software. The results are shown in Table 13. Spheres with unclear contours, overlapping spheres, and spheres whose contours could not be accurately recognized were excluded from the analysis target. FIG. 16 shows images in green that show the spheres used in the analysis.

TABLE 13

| | sphere average diameter (μm) | | |
|---|---|---|---|
| | | after enzyme treatment | |
| | after | and cell staining | |
| | pre-treatment | PBS | TrypLE |
| Comparative Example 2 | 406.0 | 419.0 | 334.5 |
| Comparative Example 3 | 463.5 | 476.0 | 450.8 |
| Example 3 | 597.7 | 609.0 | 375.7 |

As shown in Table 13, even though the spheres of Example 3 had a larger average diameter after pre-treatment as compared with Comparative Examples 2 and 3, as shown in FIG. 15, the outline of the spheres of Example 3 disappeared due to the enzyme treatment and pipetting and they were dispersed into single cells. On the other hand, the spheres of Comparative Examples 2 and 3 were not completely dispersed into single cells. In addition, the dispersed single cells in Example 3 were fluorescently stained, which clarifies that they were viable cells. The above results suggest that the spheres formed using substrate 1 may have higher dispersibility into single cells than the spheres formed without using the substrate.

### [Experimental Example 12] Dispersion of Spheres Using Cell Dispersion Tool

Human umbilical cord-derived mesenchymal stem cells (manufactured by PromoCell, #C-12971) were adhesion cultured for 3 days on a 15 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 25 mL of a medium composition in which the substrate of Preparation Example 1 was added to mesenchymal stem cell growth medium 2 to a final concentration 0.05% (w/v), such that the seeding concentration was 3×10⁴ cells/mL, and cultured for 10 days under stirring conditions of 25 rpm in a CO₂ incubator (37° C., 5% CO₂). A 30 mL single-use reactor (manufactured by ABLE Corporation, #BWV-S03A) as a culture container and an exclusive magnetic stirrer (manufactured by ABLE Corporation, #BWS-S03N0S-6) were used. On days 4 and 7 of culture, the culture container was allowed to stand for 10 min, and half of the culture supernatant was replaced with the medium.

### (Separation of Spheres)

On day 10 of culture, the entire amount of the culture medium was passed through a cell strainer with a pore size of 200 μm (manufactured by pluriSelect, #43-50200-03), it was washed with 30 mL of D-PBS(−), the mesh was turned upside down to collect the spheres trapped on the mesh with 10 mL of the D-PBS(−).

### (Enzyme Treatment)

554 μL of Liberase (registered trade mark) TM Research Grade (manufactured by Merck, #5401119001) solution dissolved in D-PBS(−) at a final concentration of 13 U/mL, 2 mL of TrypLE (registered trade mark) Select Enzyme (10×), no phenol red (manufactured by Thermo Fisher, #A1217701), and 17446 μL of D-PBS(−) were mixed to prepare 20 mL of an enzyme solution. The separated spheres were centrifuged (400×g, 3 min, Deccel mode), the supernatant was removed, and the spheres were suspended in the enzyme solution heated to 37° C. and transferred to a cell dispersion tool (manufactured by ABLE Corporation). A cell dispersion tool was set in a high-rotation stirrer with a temperature control function for dispersion tool (manufactured by ABLE Corporation) heated to 37° C., and the cells were dispersed at 1200 rpm.

### (Microscopic Observation)

At the time points of 0, 3, 5, 10, 15, 20, and 30 min of the enzyme treatment, 0.5 mL of the uniformly suspended suspension was transferred into a 12-well plate (manufactured by Corning, #351143), and observed under an inverted microscope (manufactured by Olympus Corporation, #IX73). The scale bar indicates 500 μm.

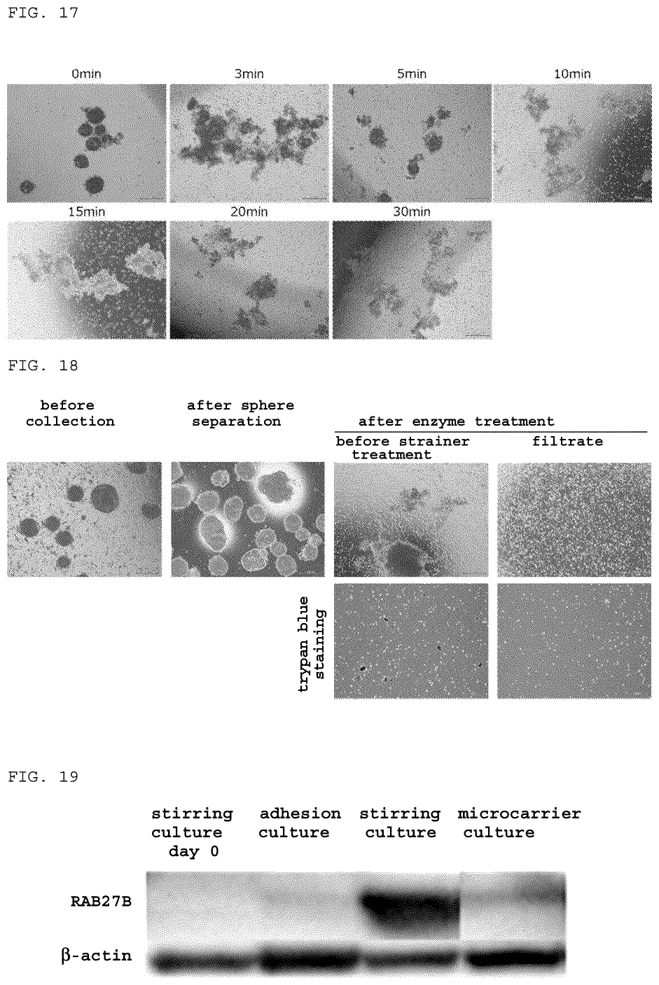

As shown in FIG. 17, the spheres were disintegrated over time and single cells were released. From the above results, it was suggested that spheres formed using the substrate can be isolated as single cells.

### [Experimental Example 13] Dispersion of Spheres in Reactor

A medium containing mesenchymal stem cell growth medium 2 (manufactured by PromoCell, #C-28009) and Penicillin-Streptomycin Solution (×100) (manufactured by FUJIFILM Wako Pure Chemical Corporation, #168-23191) was added to UniVessel (registered trade mark) Glass1 L (manufactured by Sartorius) sterilized by autoclaving (121° C., 20 min), connected to BIOSTAT (registered trade mark) B-DCU (manufactured by Sartorius), and the medium was then conditioned for 30 min under the conditions of compression air 130 ccm and CO₂ 6 ccm, 37° C., 60 rpm. Human umbilical cord-derived mesenchymal stem cells (manufactured by PromoCell, #C-12971) were adhesion cultured for 3 days on a 15 cm dish (manufactured by Corning, #430167) using mesenchymal stem cell growth medium 2. Thereafter, the cells were detached using DetachKit (manufactured by PromoCell, #C-41210), and added at 3×10⁴ cells/mL seeding concentration to a medium that had been conditioned in advance so that the final concentration of the substrate of Preparation Example 1 was 0.05% (w/v). The total amount of the medium was 450 mL. Culture was performed with stirring for 10 days under the conditions of compression air 130 ccm and CO₂ 6 ccm, 37° C., 60 rpm. On days 4 and 7 of culture, half of the cell suspension was collected in the reactor, centrifuged (300×g, 3 min, Deccel mode), the supernatant was removed, the cells were suspended in a new medium and returned to the reactor, whereby the medium was replaced.

### (Separation of Spheres)

On day 10 of culture, 50 mL of the culture medium was passed through a cell strainer with a pore size of 200 μm (manufactured by pluriSelect, #43-50200-03), and washed with 40 mL of HBSS(−) (manufactured by Thermo Fisher, #14175095). The mesh was turned upside down and HBSS(−) was added to collect the spheres trapped on the mesh. This operation was repeated to collect the spheres from 340 mL of the culture medium.

### (Enzyme Treatment)

1385 μL of Liberase (registered trade mark) TM Research Grade (manufactured by Merck, #5401119001) solution dissolved in HBSS(−) at a final concentration of 13 U/mL, 5 mL of TrypLE (registered trade mark) Select Enzyme (10×), no phenol red (manufactured by Thermo Fisher, #A1217701), and 43615 μL of HBSS(−) were mixed to prepare 50 mL of an enzyme solution. The separated spheres were centrifuged (400×g, 3 min, Deccel mode), the supernatant was removed, and the spheres were suspended in the enzyme solution heated to 37° C. and transferred to a 100 mL single-use reactor (manufactured by ABLE Corporation, #BWV-S10A). The suspension was placed on a 6-position program stirrer (manufactured by WakenBtech Co., Ltd, #WKN-1106-P) and treated at 150 rpm for 25 min in a CO₂ incubator (37° C., 5% CO₂) to disperse the cells. The suspension after dispersion was passed through a cell strainer (manufactured by Nissan Chemical Corporation) with a pore size of 65 μm, the substrate was removed, and a filtrate was obtained.

### (Microscopic Observation)

0.5 mL of the uniformly suspended culture medium at each stage was transferred into a 12-well plate (manufactured by Corning, #351143), and observed under an inverted microscope (manufactured by Olympus Corporation, #IX73). Before and after cell strainer treatment, dead cells and the substrates were specifically stained with trypan blue solution (manufactured by FUJIFILM Wako Pure Chemical Corporation, #207-17081), then added to counting slides (manufactured by Bio-Rad, #1450011) and similarly observed. The acquired image is shown in FIG. 18. The scale bar indicates 500 μm.

As shown in FIG. 18, fine substrates were removed by sphere separation, and enzyme treatment in the bioreactor disintegrated the spheres and single cells were released. Furthermore, the substrates were removed by treating using the cell strainer. The above results suggest that spheres formed using a substrate can be dispersed into single cells in a bioreactor, and that the substrate can be removed using a cell strainer.

### [Experimental Example 14] Gene Expression Analysis of Mesenchymal Stem Cells Cultured Under Suspending and Stirring Conditions

Human umbilical cord-derived mesenchymal stem cells (manufactured by PromoCell, #C-12971) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210). The obtained cells were added to 30 mL of a medium composition in which the substrate of Preparation Example 1 was added to mesenchymal stem cell growth medium 2 to a final concentration of 0.05% (w/v), such that the seeding concentration was 3×10⁴ cells/mL, and subjected to stirring culture in a CO₂ incubator (37° C., 5% CO₂). As a culture container, 30 mL single-use reactor (manufactured by ABLE Corporation, #BWV-S03A) was used, and constant stirring was performed at 25 rpm using an exclusive magnetic stirrer (manufactured by ABLE Corporation, #BWS-S03N0S-6). On day 4 of culture, the culture container was left standing for 10 min, and half of the culture supernatant was replaced with the medium. As a comparison target, cells were seeded in a 6-well adhesive culture plate (#3516, manufactured by Corning) at a density of 8×10⁴ cells/well/2 mL and adhesion cultured. On day 4 of culture, the cells were detached using Detach Kit (manufactured by PromoCell, #C-41210), and the cells were seeded at 1×10⁵ cells/well/2 mL, and adhesion cultured for 3 days more.

### (Gene Expression Analysis)

On days 0 and 7 of culture, the cells were recovered, and RLT solution (300 μL, RNeasy mini kit (manufactured by QIAGEN, #74106)) was added to give an RNA extraction solution. To the RNA extraction solution was added 70% ethanol (300 μL), and the mixture was added to RNeasy spin column and centrifuged at 8000×g for 15 sec. Successively, 700 μL of RW1 solution was added to RNeasy spin column, and centrifuged at 8000×g for 15 sec. Successively, 500 μL of RPE solution was added, and centrifuged at 8000×g for 15 sec. Furthermore, 500 μL of RPE solution was added, and centrifuged at 8000×g for 2 min. RNase-free solution was added to RNAs remaining in the RNeasy spin column to elute them. Then, cDNAs were synthesized from the obtained RNAs by using PrimeScript RT reagent Kit (Perfect Real Time) (manufactured by Takara Bio Inc., #RR037A). Using the synthesized cDNAs, Premix EX Taq (Perfect Real Time) (manufactured by Takara Bio Inc., #RR039A), and Taq man Probe (manufactured by Applied Bio Systems), real-time PCR was performed. As the Taq man Probe (manufactured by Applied Bio Systems), Hs00190284_m1 for TSPAN7, Hs00412974_m1 for MFAP4, Hs00892618_m1 for CD55, Hs00173566_m1 for GPX3, Hs01110250_m1 for HMOX1, Hs00188156_m1 for RAB27B, Hs00369211_m1 for IL33, and Hs99999905_m1 for GAPDH were used. As the instrument, real-time PCR7500 was used. In the analysis, relative values obtained by amending the values of each target gene with the values of PPIA were calculated. The results are shown in Table 14.

TABLE 14

| | day7 | |
|---|---|---|
| | adhesion | suspend and stirring |
| CD55 | 1.28 | 2.05 |
| HMOX1 | 1.29 | 2.57 |
| TSPAN7 | 1.09 | 19.97 |
| RAB27B | 1.25 | 3.8 |
| IL33 | 2.27 | 23.49 |
| GPX3 | 0.56 | 2.18 |
| MFAP4 | 1.18 | 17.83 |

As shown in Table 14, it was found that the expression of CD55, HMOX1, TSPAN7, RAB27B, IL33, GPX3, and MFAP4 was promoted in mesenchymal stem cells suspension cultured with stirring as compared with adhesion cultured mesenchymal stem cells.

### [Experimental Example 15] Examination of the Effect of Stirring Culture on the Production Amount of Extracellular Vesicles—1

Human umbilical cord-derived mesenchymal stem cells (manufactured by PromoCell, #C-12971) and human adipose-derived mesenchymal stem cell (manufactured by CellSource Co., Ltd., #0111201) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210). The obtained cells were added to 30 mL of a medium composition in which the substrate of Preparation Example 1 was added to mesenchymal stem cell growth medium 2 to a final concentration of 0.05% (w/v), such that the seeding concentration was 3×10⁴ cells/mL, and subjected to stirring culture in a CO₂ incubator (37° C., 5% CO₂) As a culture container, 30 mL single-use reactor (manufactured by ABLE Corporation, #BWV-S03A) was used, and constant stirring was performed at 25 rpm (human umbilical cord-derived mesenchymal stem cell) and 40 rpm (human adipose-derived mesenchymal stem cell) using an exclusive magnetic stirrer (manufactured by ABLE Corporation, #BWS-S03N0S-6) (stirring culture group). On day 3 of culture, the culture container was left standing for 10 min, half of the culture supernatant was replaced with a medium, and the culture was continued until day 7. On day 7 of culture, the culture medium was transferred to a 50 mL centrifuge tube, and then centrifuged at 300×g for 3 min to remove the medium. Then, 30 mL of D-PBS was added to the cells and the mixture was centrifuged at 300×g for 3 min to remove D-PBS. After performing the same operation again, 10% Fetal Bovine Serum, exosome-depleted (manufactured by Gibco, A2720801)-containing D-MEM (high glucose) (containing L-glutamine, phenol red, and sodium pyruvate) (manufactured by FUJIFILM Wako Pure Chemical Corporation, #043-30085) was added by 20 mL, and the cells were seeded in T75 Nunclon Sphera EasYFlask (manufactured by Thermo Fisher, #174952) and cultured for 2 days in a CO₂ incubator (37° C., 5% CO₂). Two days later, the culture supernatant was collected. At the time of cell seeding, on day 7 and day 9 of culture, 250 μL of the cell suspension was taken, an equal amount of CellTiter-Glo (registered trademark) Luminescent Cell Viability Assay (manufactured by Promega) was added, and luminescence intensity was measured using Enspire (manufactured by PerkinElmer), whereby the number of cells was determined at each time point. As a comparison target, cells were seeded in a 100 mm dish (manufactured by Corning, #430167) at 9×10⁵ cells/10 mL/dish and adhesion cultured (adhesion culture group). On day 2 of culture, the medium was removed and the operation of adding and removing 30 mL of D-PBS was repeated twice. Then, 10 mL of 10% Fetal Bovine Serum, exosome-depleted-containing D-MEM (high glucose) (containing L-glutamine, phenol red, and sodium pyruvate) was added, and the cells were cultured for 2 days in a CO₂ incubator (37° C., 5% CO₂). Two days later, the culture supernatant was collected, and the cells were collected using DetachKit, and the number of cells was counted.

### (Collection of Extracellular Vesicles and Measurement of Particle Number by Ultracentrifugation Method)

The collected culture supernatant was centrifuged at 2000×g for 10 min, and the supernatant was collected and then passed through a 0.22 μm filter (manufactured by Millipore, #SLGSR33SB). The treated culture supernatant was added to a UC tube (manufactured by Beckman Coulter, #344059), set in SW41Ti (manufactured by Beckman Coulter), and centrifuged using Optima L-90K under the conditions of 35000 rpm at 4° C. for 70 min. After centrifugation, the supernatant was removed, 10 mL of D-PBS was added to the UC tube, and the mixture was centrifuged under the conditions of 35000 rpm at 4° C. for 70 min. After centrifugation, the supernatant was removed and suspended in 100 μL of D-PBS. Regarding the collected extracellular vesicles, the number of particles was measured using ZetaView (manufactured by Particle Metrix). Furthermore, the amount of extracellular vesicles produced per unit cell was calculated by dividing the number of particles obtained by the number of cells at the time when the culture supernatant was obtained. The results are shown in Table 15.

TABLE 15

| | | number of | extracellular vesicles |
|---|---|---|---|
| | | extracellular | produced from unit |
| | culture | vesicles | cells (10⁹ particles/ |
| cell type | conditions | (10⁹ particles/ml) | ml/10⁶ cells) |
| umbilical | adhesion | 13 | 0.8 |
| cord-derived | culture | | |
| mesenchymal | stirring | 75 | 10.7 |
| stem cell | culture | | |
| adipose-derived | adhesion | 34 | 1.9 |
| mesenchymal | culture | | |
| stem cell | stirring | 36 | 2.9 |
| | culture | | |

As shown in Table 15, in both umbilical cord-derived and adipose-derived mesenchymal stem cells, the amount of extracellular vesicles and the amount of extracellular vesicles obtained per unit cell were greater in the stirring culture than in the adhesion culture.

### (Measurement of Exosome Marker CD63 by ELISA)

For detection of CD63, PS Capture (trademark) exosome ELISA kit (anti-mouse IgG POD) (manufactured by FUJIFILM Wako Pure Chemical Corporation, #297-79201) was used. The reaction/washing solution (1×) was prepared by diluting the Reaction/Washing Buffer (10×) 10 times with purified water, and adding a 1/100th amount of Exosome Binding Enhancer (100×) to the obtained Reaction/Washing Buffer(1×). Each culture medium was diluted 500 times with reaction/wash solution (1×). After washing the Exosome Capture 96-well plate three times with 300 μL of reaction/washing solution (1×), 500-fold diluted extracellular vesicles were added to each well and reacted at room temperature for 2 hr while shaking with a microplate shaker. After completion of the reaction, the reaction mixture was discarded, and each well was washed 3 times with 300 μL of reaction/washing solution (1×). 100 μL of Control Primary Antibody Anti-CD63(×100) diluted 1000 times with the reaction/washing solution (1×) was added and the mixture was reacted for 1 hr at room temperature while shaking using a microplate shaker. After completion of the reaction, the reaction mixture was discarded, and each well was washed 3 times with 300 μL of reaction/washing solution (1×). 100 μL of Secondary Antibody HRP-conjugated Anti-mouse IgG(100×) diluted 1000 times with the reaction/washing solution (1×) was added and the mixture was reacted for 1 hr at room temperature while shaking using a microplate shaker. After completion of the reaction, the reaction mixture was discarded, and each well was washed 5 times with 300 μL of reaction/washing solution (1×). 100 μL of TMB Solution was added and the mixture was shaken for 1 min using a microplate shaker and reacted at room temperature for 30 min. After completion of the reaction, 100 μL of Stop Solution was added and the mixture was shaken for 5 sec using a microplate shaker. The absorbance at 450 nm was measured using Enspire (manufactured by PerkinElmer). The results are shown in Table 16.

TABLE 16

| cell type | culture conditions | Abs |
|---|---|---|
| umbilical cord-derived | adhesion culture | 1.63 |
| mesenchymal stem cell | stirring culture | 2.15 |
| adipose-derived | adhesion culture | 1.41 |
| mesenchymal stem cell | stirring culture | 1.95 |
| D-PBS | | 0.24 |

As shown in Table 16, even though CD63 expression was observed under all conditions, a stronger signal was obtained in the stirring culture than in the adhesion culture. From the above, it was shown that suspension culture with stirring can enhance the production of exosomes in mesenchymal stem cells.

### [Experimental Example 16] Examination of the Effect of Stirring Culture on the Production Amount of Extracellular Vesicles—2

Human adipose-derived mesenchymal stem cell (manufactured by CellSource Co., Ltd., #0111201) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), suspended in 30 mL of a medium composition in which the substrate of Preparation Example 1 was added to mesenchymal stem cell growth medium 2 to a final concentration 0.05% (w/v), such that the seeding concentration was 3×10⁴ cells/mL, and stirring-cultured in a CO₂ incubator (37° C., 5% CO₂). A 30 mL single-use reactor (manufactured by ABLE Corporation, #BWV-S03A) was used as a culture container, an exclusive magnetic stirrer (manufactured by ABLE Corporation, #BWS-S03N0S-6) was used, and constant stirring was performed at 50 rpm (stirring culture group). As a comparison target of stirring culture, the cells were suspended in 30 mL of mesenchymal stem cell growth medium 2 containing 600 mg of Corning (registered trade mark) Low Concentration Synthemax (registered trade mark) II Microcarriers (manufactured by Corning, #3781), such that the seeding concentration was 3×10⁴ cells/mL, and stirring-cultured in a CO₂ incubator (37° C., 5% CO₂). As a culture container, 30 mL single-use reactor was used. For stirring, using an exclusive magnetic stirrer, the cells were allowed to stand for 59 min, stirred for 1 min at 55 rpm, which was repeated 10 times, and then constantly stirred at 55 rpm (microcarrier culture group). On day 4 of culture, the above-mentioned culture medium was transferred to a 50 mL centrifuge tube, and then centrifuged at 300×g for 3 min to remove the medium. Then, 30 mL of D-PBS was added and the mixture was centrifuged at 300×g for 3 min and D-PBS was removed. After performing the same operation once again, 10% Fetal Bovine Serum, exosome-depleted (manufactured by Gibco, A2720801)-containing D-MEM (high glucose) (containing L-glutamine, phenol red, and sodium pyruvate) (manufactured by FUJIFILM Wako Pure Chemical Corporation, #043-30085) was added by 30 mL, re-seeded in the exclusive magnetic stirrer and constant stirring was performed for 2 days at 50 rpm or 55 rpm. Two days later, the culture supernatant was collected. At the time of cell seeding and on day 6 of culture, 250 μL of the cell suspension was taken, an equal amount of CellTiter-Glo (registered trademark) Luminescent Cell Viability Assay (manufactured by Promega) was added, and luminescence intensity was measured using Enspire (manufactured by PerkinElmer), whereby the number of cells was determined at each time point. For adhesion culture, cells were seeded in a 100 mm dish (manufactured by Corning, #430167) at 9×10⁵ cells/10 mL/dish and adhesion cultured (adhesion culture group). On day 2 of culture, the medium was removed and the operation of adding and removing 30 mL of D-PBS was repeated twice. Then, 10 mL of 10% Fetal Bovine Serum, exosome-depleted-containing D-MEM (high glucose) (L-glutamine, phenol red, containing sodium pyruvate) was added, and the cells were cultured for 2 days in a CO₂ incubator (37° C., 5% CO₂)-Two days later, the culture supernatant was collected, and the cells were collected using DetachKit, and the number of cells was counted. On day 0, day 4 (adhesion culture), and day 6 (stirring culture and microcarrier culture), the cells were collected, RLT solution (300 μL) (RNeasy mini kit (manufactured by QIAGEN, #74106) was added to give an RNA extraction solution. In addition, on day 0, day 4 (adhesion culture), and day 6 (stirring culture and microcarrier culture), using 150 μL of RIPA buffer (manufactured by FUJIFILM Wako Pure Chemical Corporation, #182-02451) containing 1×Halt (trademark) Protease and Phosphatase Inhibitor Single-Use Cocktail (100×), a whole cell lysate was prepared.

### (Collection of Extracellular Vesicles and Measurement of Particle Number by Ultracentrifugation Method)

The collected culture supernatant was centrifuged at 2000×g for 10 min, and the supernatant was collected and then passed through a 0.22 μm filter (manufactured by Millipore, #SLGSR33SB). The treated culture supernatant was added to a UC tube (manufactured by Beckman Coulter, #344059), set in SW41Ti (manufactured by Beckman Coulter), and centrifuged using Optima L-90K under the conditions of 35000 rpm at 4° C. for 70 min. After centrifugation, the supernatant was removed, 10 mL of D-PBS was added to the UC tube, and the mixture was centrifuged under the conditions of 35000 rpm at 4° C. for 70 min. After centrifugation, the supernatant was removed. The adhesion culture group was suspended in 50 μL of D-PBS and the microcarrier culture and stirring culture group was suspended in 100 μL of D-PBS. Regarding the collected extracellular vesicles, the number of particles was measured using ZetaView (manufactured by Particle Metrix). Regarding the collected extracellular vesicles, the number of particles was measured using ZetaView (manufactured by Particle Metrix). Furthermore, the amount of extracellular vesicles produced per unit cell was calculated by dividing the number of particles obtained by the number of cells at the time when the culture supernatant was obtained. The results are shown in Table 17.

TABLE 17

| culture | | 10⁹ particles/ml/ |
|---|---|---|
| conditions | 10⁹ particles/ml | 10⁶ cells |
| adhesion culture | 2.2 | 0.1 |
| microcarrier | 15 | 0.8 |
| culture | | |
| stirring culture | 33 | 5.3 |

As shown in Table 17, the number of extracellular vesicles and the amount of extracellular vesicles per unit cell were greater in the stirring culture than in the adhesion culture and microcarrier culture.

### (Measurement of Extracellular Vesicle Marker by ELISA)

For detection of CD63, PS Capture (trademark) exosome ELISA kit (anti-mouse IgG POD) (manufactured by FUJIFILM Wako Pure Chemical Corporation, #297-79201) was used. The reaction/washing solution (1×) was prepared by diluting the Reaction/Washing Buffer (10×) 10 times with purified water, and adding a 1/100th amount of Exosome Binding Enhancer (100×) to the obtained Reaction/Washing Buffer(1×). Considering the amount of medium used and the amount of solution suspended after ultracentrifugation, 400-fold diluted extracellular vesicle solution was used in the adhesion culture group and 600-fold diluted reaction/washing solution (1×) was used in the microcarrier culture and stirring culture groups. After washing the Exosome Capture 96-well plate three times with 300 μL of reaction/washing solution (1×), 100 μL of the diluted extracellular vesicle solution was added to each well and reacted at room temperature for 2 hr while shaking with a microplate shaker. After completion of the reaction, the reaction mixture was discarded, and each well was washed 3 times with 300 μL of reaction/washing solution (1×). 100 μL of Control Primary Antibody Anti-CD63(×100) diluted 1000 times with the reaction/washing solution (1×) was added and the mixture was reacted for 1 hr at room temperature while shaking using a microplate shaker. After completion of the reaction, the reaction mixture was discarded, and each well was washed 3 times with 300 μL of reaction/washing solution (1×). 100 μL of Secondary Antibody HRP-conjugated Anti-mouse IgG(100×) diluted 1000 times with the reaction/washing solution (1×) was added and the mixture was reacted for 1 hr at room temperature while shaking using a microplate shaker. After completion of the reaction, the reaction mixture was discarded, and each well was washed 5 times with 300 μL of reaction/washing solution (1×). 100 μL of TMB Solution was added and the mixture was shaken for 1 min using a microplate shaker and reacted at room temperature for 30 min. After completion of the reaction, 100 μL of Stop Solution was added and the mixture was shaken for 5 sec using a microplate shaker. The absorbance at 450 nm was measured using Enspire (manufactured by PerkinElmer). The background absorbance was subtracted from each group and the value was calculated as ΔAbs. The results are shown in Table 18.

TABLE 18

| | ΔAbs |
|---|---|
| adhesion culture | 0.314 |
| microcarrier culture | 0.534 |
| stirring culture | 0.619 |

As shown in Table 18, even though CD63 expression was observed under all conditions, the strongest signal was obtained in the stirring culture.

### (Confirmation of RAB27B Expression Level by Gene Expression Analysis)

To the RNA extraction solution was added 70% ethanol (300 μL), and the mixture was added to RNeasy spin column and centrifuged at 8000×g for 15 sec. Successively, 700 μL of RW1 solution was added to RNeasy spin column, and centrifuged at 8000×g for 15 sec. Successively, 500 μL of RPE solution was added, and centrifuged at 8000×g for 15 sec. Furthermore, 500 μL of RPE solution was added, and centrifuged at 8000×g for 2 min. RNase-free solution was added to RNAs remaining in the RNeasy spin column to elute them. Then, cDNAs were synthesized from the obtained RNAs by using PrimeScript RT reagent Kit (Perfect Real Time) (manufactured by Takara Bio Inc., #RR037A). Using the synthesized cDNAs, Premix EX Taq (Perfect Real Time) (manufactured by Takara Bio Inc., #RR039A), and Taq man Probe (manufactured by Applied Bio Systems), real-time PCR was performed. As the Taq man Probe (manufactured by Applied Bio Systems), Hs00188156_m1 for RAB27B, and Hs99999905_m1 for GAPDH were used. As the instrument, QuantStudio4 (manufactured by Thermo Fisher) was used. In the analysis, relative values obtained by amending the values of each target gene with the values of GAPDH were calculated and compared.

TABLE 19

| | Relative |
|---|---|
| day0 | 0.03 |
| adhesion culture | 0.26 |
| microcarrier culture | 0.61 |
| stirring culture | 1.16 |

As shown in Table 19, an increase in the mRNA expression level of RAB27B was observed in stirring culture.

### (Confirmation of Expression Variation of RAB27B Protein)

The electrophoresis tank was filled with Ezrun C+ solution (manufactured by ATTO, #2332320) as a buffer. E-T12.5 L e-PAGEL 12.5% (manufactured by ATTO, #2331820) was set as a gel for electrophoresis, and each sample was loaded at 12 μg/lane. Electrophoresis was performed at 100V for 70 min. After electrophoresis, transfer to the membrane was performed for 7 min under the conditions of 1.3 A and 25 V using Trans-Blot Turbo Mini PVDF Transfer Pack (manufactured by Bio-Rad, #1704156). After transfer, the membrane was immersed in a TBS-T solution prepared using Tris Buffered Saline with Tween (registered trade mark) 20 (TBS-T) Tablets, pH 7.6 (manufactured by Takara Bio Inc., #T9142) and shaken at room temperature for 1 hr. Thereafter, it was immersed in PVDF Blocking Reagent for Can Get Signal (registered trade mark) (manufactured by TOYOBO, #NYPBR01) and shaken at room temperature for 3 hr. The membrane was immersed in TBS-T solution, shaken once for 15 minutes and twice for 5 min, immersed in Anti RAB27B Human (Rabbit) Unlabeled mAb (manufactured by Peprotech, #13412-1-AP) 2000-fold diluted with Can Get Signal Solution 1 (manufactured by TOYOBO, #NKB-201), and β-Actin (D6A8) Rabbit mAb (manufactured by Cell Signaling TECHNOLOGY, #8457) diluted 2000-fold, and shaken at 4° C. overnight. The next day, the membrane was immersed in TBS-T solution, shaken 3 times for 20 minutes, and then immersed in Anti-Rabbit IgG, HRP-Linked Whole Ab Donkey (manufactured by Cytiva, #NA934-1ML) diluted 5000-fold with Can Get Signal Solution 2 (manufactured by TOYOBO, #NKB-301), and shaken at room temperature for 1 hr. The membrane was immersed in TBS-T solution, shaken once for 15 minutes and once for 1 hr, and emitted using ImmunoStar (registered trade mark) Zeta (manufactured by FUJIFILM Wako Pure Chemical Corporation, #297-72403). Detection was performed using ChemiDoc XRS Plus (manufactured by Bio-Rad). The results are shown in FIG. 19.

As shown in Table 19, an increase in the RAB27B protein expression level was observed in stirring culture.

### [Experimental Example 17] Signal Pathway Analysis of the Mesenchymal Stem Cells Cultured Using the Method of the Present Invention

Human adipose-derived mesenchymal stem cells (manufactured by CellSource Co., Ltd., #0111201) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210). The detached cells were suspended in 30 mL of mesenchymal stem cell growth medium 2 containing the substrate of Preparation Example 1 (final concentration 0.05% (w/v)), such that the seeding concentration was 3×10⁴ cells/mL, and stirring-cultured in a CO₂ incubator (37° C., 5% CO₂). A 30 mL single-use reactor (manufactured by ABLE Corporation, #BWV-S03A) was used as a culture container, an exclusive magnetic stirrer (manufactured by ABLE Corporation, #BWS-S03N0S-6) was used for stirring, and constant stirring was performed at 50 rpm (stirring culture group). On day 3 of culture, the culture container was left standing for 10 min, half of the culture supernatant was replaced with a medium, and the culture was continued until day 7. As a control, adhesion culture was performed for 3 days on a 10 cm dish (manufactured by Corning, #430167) (adhesion culture group). Nuclear fractions were obtained from adhesion cultured and stirring cultured cells on days 3 and 7 of culture using Nuclear Extraction Kit (manufactured by Raybio, #NE-50).

(Confirmation of Expression of NFE2 L2 (Also Referred to as “NRF2”), P65, and Phosphorylated P65 (p-P65) Proteins by Western Blotting)

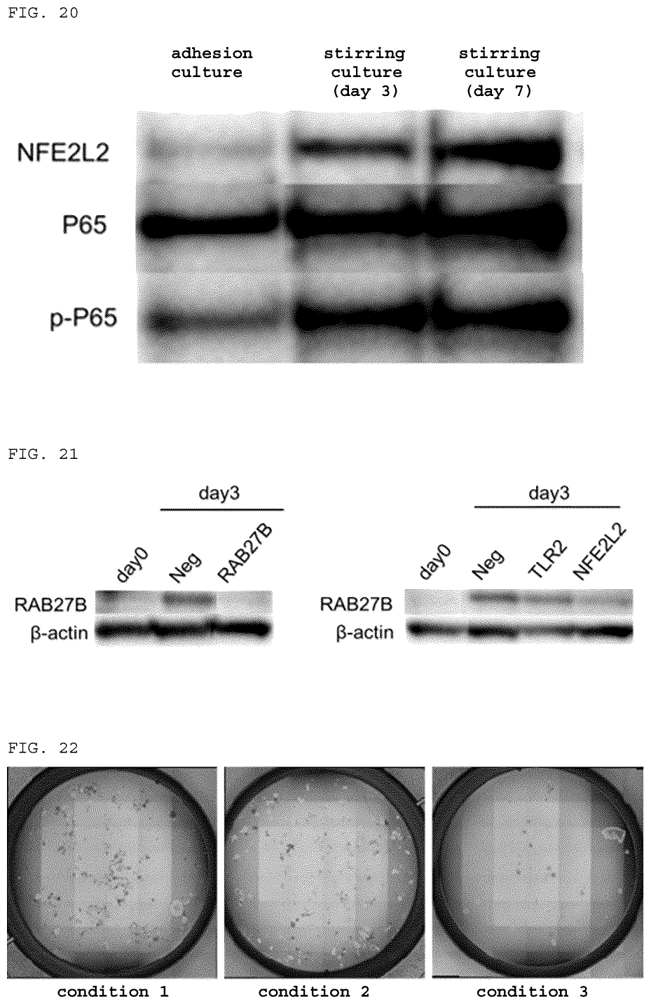

The electrophoresis tank was filled with Ezrun C+ solution (manufactured by ATTO, #2332320) as a buffer. E-T12.5 L e-PAGEL 12.5% (manufactured by ATTO, #2331820) was set as a gel for electrophoresis, and each sample was loaded at 12 g/lane. Electrophoresis was performed at 100V for 70 min. Transfer to the membrane was performed for 7 min under the conditions of 1.3 A and 25 V using Trans-Blot Turbo Mini PVDF Transfer Pack (manufactured by Bio-Rad, #1704156). After transfer, the membrane was immersed in a TBS-T solution prepared using Tris Buffered Saline with Tween (registered trade mark) 20 (TBS-T) Tablets, pH 7.6 (manufactured by Takara Bio Inc., #T9142) and shaken at room temperature for 1 hr. Thereafter, it was immersed in PVDF Blocking Reagent for Can Get Signal (registered trade mark) (manufactured by TOYOBO, #NYPBR01) and shaken at room temperature for 3 hr. The membrane was immersed in TBS-T solution, shaken once for 15 min and twice for 5 min, immersed in NRF2 (D1Z9C) XPR Rabbit mAb (manufactured by Cell Signaling TECHNOLOGY, #12721) 1000-fold diluted with Can Get Signal Solution 1 (manufactured by TOYOBO, #NKB-201), 1000-fold diluted Anti p65; RELA, Human (Rabbit) Unlabeled (manufactured by Peprotech, #10745-1-AP), and 1000-fold diluted Phospho-NF-kB p65(Ser536) (93H1)Rabbit mAb (manufactured by Cell Signaling TECHNOLOGY, #3033), and shaken at 4° C. overnight. The membrane was immersed in TBS-T solution, shaken 3 times for 20 minutes, and then immersed in Anti-Rabbit IgG, HRP-Linked Whole Ab Donkey (manufactured by Cytiva, #NA934-1ML) diluted 5000-fold with Can Get Signal Solution 2 (manufactured by TOYOBO, #NKB-301), and shaken at room temperature for 1 hr. The membrane was immersed in TBS-T solution, shaken once for 15 minutes and once for 1 hr, and emitted using ImmunoStar (registered trade mark) Zeta (manufactured by FUJIFILM Wako Pure Chemical Corporation, #297-72403). Detection was performed using ChemiDoc XRS Plus (manufactured by Bio-Rad). The results are shown in FIG. 20.

As shown in FIG. 20, it was clarified that NFE2 L2 and NF-kB subunits P65 and p-P65 in the nucleus increased in the stirring culture as compared with adhesion culture.

### [Experimental Example 18] Analysis of the Mechanism of Increased Production of Extracellular Vesicles Derived from Mesenchymal Stem Cells Using Stirring Culture

Human adipose-derived mesenchymal stem cells (manufactured by CellSource Co., Ltd., #0111201) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), and seeded in a 6-well cell culture plate (manufactured by Corning, #3516) to a seeding concentration of 5×10⁵ cells/1.75 mL. Simultaneously, Opti-MEM (trademark) I Reduced Serum Medium (manufactured by Thermo Fisher, #31985070) containing 7.5 μL of Lipofectamine RNAiMAX Transfection Reagent (manufactured by Thermo Fisher, #13778075), 25 pmol of Silencer Select Negative Control #1 siRNA (hereinafter sometimes referred to as “Neg”) (manufactured by Thermo Fisher, #4390843), RAB27B (#s11696), NFE2 L2 (#s9493), TLR2 (#s169) (all manufactured by Thermo Fisher) was added to each well at 250 μL/well. After one day, the medium was removed and the cells were detached using DetachKit and suspended at 3×10⁴ cells/mL in 5 mL or 30 mL of mesenchymal stem cell growth medium 2 medium composition containing the substrate of Preparation Example 1 at a final concentration of 0.05% (w/v), and stirring-cultured in a CO₂ incubator (37° C., 5% CO₂). A 5 mL single-use reactor (manufactured by ABLE Corporation, #ABBWVS05A) or 30 mL single-use reactor (manufactured by ABLE Corporation, #BWV-S03A) was used as a culture container, an exclusive magnetic stirrer (manufactured by ABLE Corporation, #ABBWBP05N0S-6 or #BWS-S03N0S-6) was used for stirring, and constant stirring was performed at 50 rpm (stirring culture group). Two days later, the entire medium containing cells and substrates was transferred to a centrifuge tube and centrifuged at 300×g for 3 min. The culture supernatant after centrifugation was collected for ELISA measurement. On days 0 and 3, the cells were recovered, and RLT solution (300 μL, RNeasy mini kit (manufactured by QIAGEN, #74106)) was added to give an RNA extraction solution. In addition, on days 0 and 3, a whole cell lysate was prepared using 150 μL of RIPA buffer (manufactured by FUJIFILM Wako Pure Chemical Corporation, #182-02451) containing 1×Halt (trademark) Protease and Phosphatase Inhibitor Single-Use Cocktail (100×).

### (Confirmation of RAB27B Expression Level by Gene Expression Analysis)

To the RNA extraction solution was added 70% ethanol (300 μL), and the mixture was added to RNeasy spin column and centrifuged at 8000×g for 15 sec. Successively, 700 μL of RW1 solution was added to RNeasy spin column, and centrifuged at 8000×g for 15 sec. Successively, 500 μL of RPE solution was added, and centrifuged at 8000×g for 15 sec. Furthermore, 500 μL of RPE solution was added, and centrifuged at 8000×g for 2 min. RNase-free solution was added to RNAs remaining in the RNeasy spin column to elute them. Then, cDNAs were synthesized from the obtained RNAs by using PrimeScript RT reagent Kit (Perfect Real Time) (manufactured by Takara Bio Inc., #RR037A). Using the synthesized cDNAs, Premix EX Taq (Perfect Real Time) (manufactured by Takara Bio Inc., #RR039A), and Taq man Probe (manufactured by Applied Bio Systems), real-time PCR was performed. As the Taq man Probe (manufactured by Applied Bio Systems), Hs00188156_m1 for RAB27B, and Hs99999905_m1 for GAPDH were used. As the instrument, QuantStudio4 (manufactured by Thermo Fisher) was used. In the analysis, relative values obtained by amending the values of each target gene with the values of GAPDH were calculated and compared. The results are shown in Table 20.

TABLE 20

| | siRNA | Relative | | siRNA | Relative |
|---|---|---|---|---|---|
| day0 | — | 0.14 | day0 | — | 0.18 |
| Day3 | Neg | 4.201 | day3 | Neg | 5.17 |
| | RAB27B | 0.776 | | TLR2 | 2.09 |
| | | | | NFE2L2 | 2.23 |

As shown in Table 20, a decrease in the expression level of RAB27B mRNA due to RAB27B siRNA was confirmed in the stirring culture. Furthermore, a decrease in the expression level of RAB27B mRNA was also observed upon treatment with NFE2 L2 or TLR2 siRNA.

### (Confirmation of Expression Variation of RAB27B Protein)

The electrophoresis tank was filled with Ezrun C+ solution (manufactured by ATTO, #2332320) as a buffer. E-T12.5 L e-PAGEL 12.5% (manufactured by ATTO, #2331820) was set as a gel for electrophoresis, and each sample was loaded at 12 g/lane. Electrophoresis was performed at 100V for 70 min. After electrophoresis, transfer to the membrane was performed for 7 min under the conditions of 1.3 A and 25 V using Trans-Blot Turbo Mini PVDF Transfer Pack (manufactured by Bio-Rad, #1704156). After transfer, the membrane was immersed in a TBS-T solution prepared using Tris Buffered Saline with Tween (registered trade mark) 20 (TBS-T) Tablets, pH 7.6 (manufactured by Takara Bio Inc., #T9142) and shaken at room temperature for 1 hr. Thereafter, it was immersed in PVDF Blocking Reagent for Can Get Signal (registered trade mark) (manufactured by TOYOBO, #NYPBR01) and shaken at room temperature for 3 hr. The membrane was immersed in TBS-T solution, shaken once for 15 min and twice for 5 min, immersed in Anti RAB27B 2000-fold diluted with Can Get Signal Solution 1 (manufactured by TOYOBO, #NKB-201), Human (Rabbit) Unlabeled (manufactured by Peprotech, #13412-1-AP), and R-Actin (D6A8) Rabbit mAb (manufactured by Cell Signaling TECHNOLOGY, #8457) diluted 2000-fold, and shaken at 4° C. overnight. The next day, the membrane was immersed in TBS-T solution, shaken 3 times for 20 minutes, and then immersed in Anti-Rabbit IgG, HRP-Linked Whole Ab Donkey (manufactured by Cytiva, #NA934-1ML) diluted 5000-fold with Can Get Signal Solution 2 (manufactured by TOYOBO, #NKB-301), and shaken at room temperature for 1 hr. The membrane was immersed in TBS-T solution, shaken once for 15 minutes and once for 1 hr, and emitted using ImmunoStar (registered trade mark) Zeta (manufactured by FUJIFILM Wako Pure Chemical Corporation, #297-72403). Detection was performed using ChemiDoc XRS Plus (manufactured by Bio-Rad). The results are shown in FIG. 21.

As shown in FIG. 21, a decrease in the protein expression level of RAB27B due to RAB27B siRNA treatment was confirmed in the stirring culture. Furthermore, a decrease in the protein expression level of RAB27B was also observed upon treatment with NFE2 L2 or TLR2 siRNA.

Since the expression levels of RAB27B mRNA and protein decreased upon treatment with NFE2 L2 or TLR2 siRNA, it was considered that culturing mesenchymal stem cells on this substrate increased the expression level of RAB27B through TLR2 and NFE2 L2 and, as a result, the amount of sEV produced increased.

### (Measurement of Extracellular Vesicle Marker by ELISA)

For detection of CD63, PS Capture (trademark) exosome ELISA kit (anti-mouse IgG POD) (manufactured by FUJIFILM Wako Pure Chemical Corporation, #297-79201) was used. The reaction/washing solution (1×) was prepared by diluting the Reaction/Washing Buffer (10×) 10 times with purified water, and adding a 1/100th amount of Exosome Binding Enhancer (100×) to the obtained Reaction/Washing Buffer(1×). After washing the Exosome Capture 96-well plate three times with 300 μL of reaction/washing solution (1×), the obtained culture supernatant (100 μL) was added to each well and reacted at room temperature for 2 hr while shaking with a microplate shaker. After completion of the reaction, the reaction mixture was discarded, and each well was washed 3 times with 300 μL of reaction/washing solution (1×). 100 μL of Control Primary Antibody Anti-CD63(×100) diluted 1000 times with the reaction/washing solution (1×) was added and the mixture was reacted for 1 hr at room temperature while shaking using a microplate shaker. After completion of the reaction, the reaction mixture was discarded, and each well was washed 3 times with 300 μL of reaction/washing solution (1×). 100 μL of Secondary Antibody HRP-conjugated Anti-mouse IgG(100×) diluted 1000 times with the reaction/washing solution (1×) was added and the mixture was reacted for 1 hr at room temperature while shaking using a microplate shaker. After completion of the reaction, the reaction mixture was discarded, and each well was washed 5 times with 300 μL of reaction/washing solution (1×). 100 μL of TMB Solution was added and the mixture was shaken for 1 min using a microplate shaker and reacted at room temperature for 30 min. After completion of the reaction, 100 μL of Stop Solution was added and the mixture was shaken for 5 sec using a microplate shaker. The absorbance at 450 nm was measured using Enspire (manufactured by PerkinElmer). The background absorbance was subtracted from each group and the value was calculated as ΔAbs. The results are shown in Table 21.

TABLE 21

| siRNA | ΔAbs | siRNA | ΔAbs |
|---|---|---|---|
| Neg | 0.61 | Neg | 0.93 |
| RAB27B | 0.53 | TLR2 | 0.79 |
| | | NFE2L2 | 0.71 |

As shown in Table 21, a decrease in the absorbance of CD63 was observed by siRNA treatment of RAB27B, NFE2 L2, and TLR2.

While not bound by theory, the mesenchymal stem cells prepared by the method of the present invention are characterized in that they have the following molecular mechanisms:
↓ interaction between mesenchymal stem cells and the substrate used in the present invention
↓ activation of TLR2 signal
↓ activation of NF-κB signal and NFE2 L2 (NRF2) signal
↓ increased expression of PGE2 gene and TSG6 gene due to activation of NF-κB signal (higher functioning of MSC)
↓ increased expression of RAB27B gene due to activation of NFE2 L2 signal (increased secretion amount of extracellular vesicles from MSC)

### [Preparation Example 2] Preparation of Aqueous Dispersion Containing Chitin Nanofiber Carrying Vitronectin

A 2% by mass chitin nanofiber aqueous dispersion prepared according to the description in WO 2015/111686 was sterilized in an autoclave at 121° C. for 20 min. Thereafter, this aqueous dispersion was mixed and suspended in sterile distilled water (Otsuka Distilled Water, manufactured by Otsuka Pharmaceutical Factory Co., Ltd.) to a concentration of 1% (w/v) to produce a sterile aqueous dispersion containing chitin nanofibers. To a 1% (w/v) chitin nanofiber aqueous dispersion (5 mL) was added with a vitronectin aqueous solution containing 500 μg/mL (Gibco Vitronectin (VTN-N) Recombinant Human Protein, Truncated, manufactured by Thermo Fisher Scientific) (0.5 mL), mixed by pipetting, and then stored by standing at 4° C. overnight to produce an aqueous dispersion containing vitronectin-carried chitin nanofibers. (In the present specification, the chitin nanofiber carrying vitronectin prepared here is sometimes simply referred to as “substrate of Preparation Example 2”, “Preparation Example 2”, or “Substrate 2”).

### [Experimental Example 19] Examination of Culture and Passage Method of Mesenchymal Stem Cells Using Substrate 2 and Stirring—1

Human umbilical cord-derived mesenchymal stem cell (manufactured by PromoCell, #C-12971) were adhesion cultured for 3 days on a 10 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), and added at a seeding concentration of 1.5×10⁴ cells/mL to 30 mL of a medium composition in which the substrate of Preparation Example 2 was added to mesenchymal stem cell growth medium 2 at a final concentration of 0.01% (w/v). A 30 mL single-use reactor (manufactured by ABLE Corporation, #BWV-S03A) was used, and the cells were cultured by constant stirring at 25 rpm using an exclusive magnetic stirrer (manufactured by ABLE Corporation, #BWS-S03N0S-6) in a CO₂ incubator (37° C., 5% or 10% CO₂) for 3 days. On day 3 of culture, the cells were cultured again under the following condition 1, 2, or 3, and microscopic observation using fluorescent staining and evaluation of proliferation were performed.

### (Condition 1: Passage by the Addition of Substrate 2)

The culture suspension (3 mL) on day 3 of culture was separated and 27 mL of a medium composition in which the substrate of Preparation Example 2 was added to mesenchymal stem cell growth medium 2 to a final concentration of 0.01% (w/v). As a culture container, a 30 mL single-use reactor (manufactured by ABLE Corporation, #BWV-S03A) was used, and the cells were cultured by constant stirring at 25 rpm using an exclusive magnetic stirrer (manufactured by ABLE Corporation, #BWS-S03N0S-6) in a CO₂ incubator (37° C., 5% or 10% CO₂) for 4 days.

### (Condition 2: Passage by Physical Treatment and Addition of Substrate 2)

An autoclaved sterile PP straight coupling (manufactured by Isis Co., Ltd., #VRFC6) was connected to a 30 mL syringe (manufactured by Nipro, #8955) filled with the entire amount of the culture suspension on day 3 of culture, and a new 30 mL syringe (manufactured by Nipro, #8955) was connected to the other end. The spheroids were loosened by extruding three times from the filled syringe to the empty syringe at a rate of about 1 mL/s to apply physical shearing force. 3 mL of the obtained suspension was taken and 27 mL of a medium composition in which the substrate of Preparation Example 2 was added to mesenchymal stem cell growth medium 2 at a final concentration of 0.01% (w/v). As a culture container, a 30 mL single-use reactor (manufactured by ABLE Corporation, #BWV-S03A) was used, and the cells were cultured by constant stirring at 25 rpm using an exclusive magnetic stirrer (manufactured by ABLE Corporation, #BWS-S03N0S-6) in a CO₂ incubator (37° C., 5% or 10% CO₂) for 3 days.

(Condition 3: Culture without Addition of Substrate 2 (Comparative Example))

The culture suspension (3 mL) on day 3 of culture was separated and 27 mL of mesenchymal stem cell growth medium 2 was added. As a culture container, a 30 mL single-use reactor (manufactured by ABLE Corporation, #BWV-S03A) was used, and the cells were cultured by constant stirring at 25 rpm using an exclusive magnetic stirrer (manufactured by ABLE Corporation, #BWS-S03N0S-6) in a CO₂ incubator (37° C., 5% or 10% CO₂) for 4 days.

### (Microscopic Observation by Fluorescent Staining)

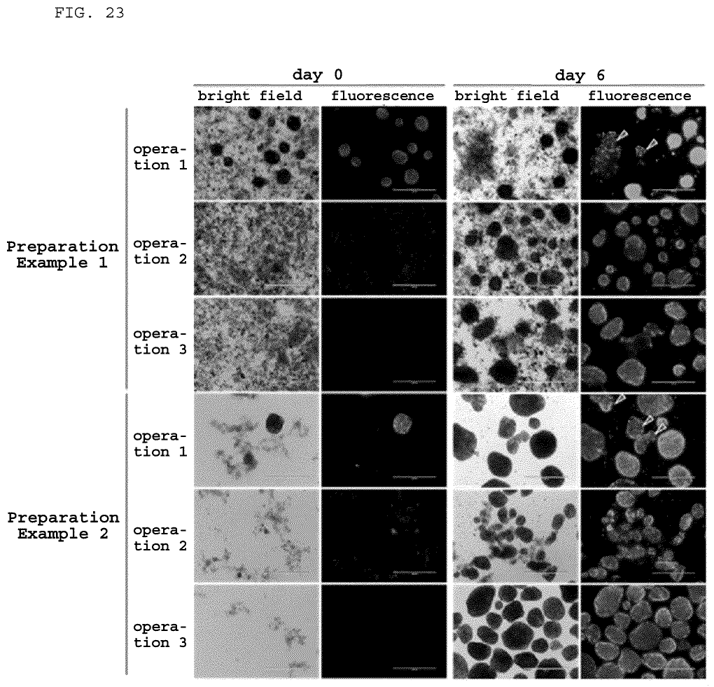

On day 4 of re-culture, 0.5 mL of the uniformly suspended culture medium was collected into a 1.5 mL tube, and after centrifugation (600×g, 3 min), the culture supernatant was removed. The cells were suspended in 1 mL of D-PBS(−) (manufactured by FUJIFILM Wako Pure Chemical Corporation, #045-29795), centrifuged (600×g, 3 min), and the culture supernatant was removed. A Calcein-AM (manufactured by DOJINDO LABORATORIES, #C326) solution (10 μL) dissolved in DMSO at a final concentration of 0.5 mg/mL was dissolved in 5 mL of D-PBS(−) (manufactured by FUJIFILM Wako Pure Chemical Corporation, #045-29795) and used as a staining solution. The cells were suspended in 1 mL of the staining solution, transferred to a 12-well plate (manufactured by Corning, #351143), and incubated in a CO₂ incubator (37° C., 5% CO₂) for 30 min. Thereafter, bright field images and viable cell-specific fluorescent staining images were obtained using a fluorescence microscope (manufactured by KEYENCE CORPORATION, BIOR EVO BZ-9000). The results are shown in FIG. 22.

As shown in FIG. 22, proliferated cells were also observed on the newly added substrates under both conditions 1 and 2. In particular, under condition 2, it was suggested that the number of spheroids increased as the loosened cells proliferated while embracing new substrates.

### (Calculation of Proliferation Rate)

On days 0 and 3 of culture and days 0 and 4 of passage, 0.5 mL of uniformly suspended culture medium was collected, and 0.5 mL of ATP reagent (CellTiter-Glo (registered trade mark) Luminescent Cell Viability Assay, manufactured by Promega) was added to each. The mixture was stirred with a vortex, allowed to stand at room temperature for 10 min, and dispensed into a white 96-well plate by 150 μL. The luminescence intensity (RLU value) was measured by Enspire (manufactured by Perkin Elmer) and the number of viable cells was measured by subtracting the luminescence value of the medium alone. The relative value when the RLU value (ATP measurement, luminescence intensity) on day 0 of culture was set to 1 was defined as the cell proliferation rate. The results are shown in Table 22.

TABLE 22

| proliferation | | | passage | passage |
|---|---|---|---|---|
| rate | day 0 | day 3 | day 0 | day 4 |
| condition 1 | 1 | 5.1 | 1 | 9.4 |
| condition 2 | 1 | 4.5 | 1 | 16.6 |
| condition 3 | 1 | 5.1 | 1 | 4.8 |

As shown in Table 22, it was confirmed that the best proliferation performance was shown under conditions in which passage culture was performed by adding substrate 2 to physically loosened spheroids as compared with condition 3 where the spheroids themselves simply continued to partially proliferate. In addition, a certain level of proliferation was also exhibited under condition 1 where subculture was performed only with the addition of substrate 2.

### [Experimental Example 20] Examination of Passage Method—2

### (Substrate of Preparation Example 1 (Substrate 1))

Human adipose tissue-derived mesenchymal stem cells (manufactured by CellSource Co., Ltd., #0111201) were adhesion cultured for 3 days on a 15 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 100 mL of a medium composition in which the substrate of Preparation Example 1 was added to mesenchymal stem cell growth medium 2 to a final concentration 0.05% (w/v), such that the seeding concentration was 1.5×10⁴ cells/mL, and cultured for 6 days under stirring conditions of 50 rpm in a CO₂ incubator (37° C., 5% CO₂). A 100 mL single-use reactor (manufactured by ABLE Corporation, #BWV-S10A) as a culture container, and an exclusive magnetic stirrer (manufactured by ABLE Corporation, #BWS-S03N0S-6) were used. In addition, after sterilizing a 1 L culture glass tank (manufactured by ABLE Corporation) with triangular blades attached thereto by autoclaving (121° C., 20 min), mesenchymal stem cell growth medium 2 and Penicillin-Streptomycin Solution (×100) (manufactured by FUJIFILM Wako Pure Chemical Corporation, #168-23191) were added, and placed in a BCP type animal cell culture device (manufactured by ABLE Corporation), and the culture medium was conditioned for 30 min under the conditions of 37° C. and 30 rpm under the control of 140 ccm of compressed air and appropriate addition of CO₂ to adjust the pH to 7.5.

### (Substrate of Preparation Example 2 (Substrate 2))

Human adipose tissue-derived mesenchymal stem cells cultured and detached using the same method as described for the substrate of Preparation Example 1 were seeded at a seeding concentration of 1.5×10⁴ cells/mL in a medium in which the substrate of Preparation Example 2 was conditioned in advance to a final concentration of 0.01% (w/v). The total amount of medium was 1000 mL. Culture was performed under control conditions similar to those for conditioning. In all cases, the culture was performed for 6 days, and on day 4 of culture, the culture container was allowed to stand for 10 min, and half of the culture supernatant was replaced with the medium.

### (Passage)

Three operations were performed on the cells on day 6 of culture using Preparation Example 1 or 2, and the culture was continued. Specifically, the cells subjected to the specific operations (operations 1 to 3) were added to 30 mL of a medium composition in which the substrate of Preparation Example 1 was added to mesenchymal stem cell growth medium 2 at a final concentration of 0.05% (w/v) or the substrate of Preparation Example 2 was added thereto at a final concentration of 0.01% (w/v), and cultured for 6 days in a CO₂ incubator (37° C., 5% CO₂) under stirring conditions of 50 rpm. A 30 mL single-use reactor (manufactured by ABLE Corporation, #BWV-S03A) as a culture container, and an exclusive magnetic stirrer (manufactured by ABLE Corporation, #BWS-S03N0S-6) were used. On day 4 of culture, the culture container was allowed to stand for 10 min, and half of the culture supernatant was replaced with the medium.

Cells subjected to operation 1: Cells in which 10 mL of cell suspension was dispensed into a centrifuge tube, left to stand for 10 min, and then the supernatant was removed.

Cells subjected to operation 2: Cells obtained by the following. 10 mL of the cell suspension was dispensed into a centrifuge tube, and after centrifugation (300×g, 3 min, Decel mode), the supernatant was removed. Thereafter, 9773 μL of D-PBS(−) and 277 μL of an enzyme solution in which Liberase MNP-S 35 mg (manufactured by CustomBiotech, #05578566001) was dissolved in 14 mL of D-PBS(−) were added, and the mixture was incubated for 30 min in a 37° C. water bath, and pipetting was performed 20 times every 10 min. Thereafter, 10 mL of mesenchymal stem cell growth medium 2 was added, centrifuged (300×g, 3 min, Decel mode), and the supernatant was removed.

Cells subjected to operation 3: Cells obtained by the following. After passing 100 mL of the cell suspension through a cell strainer with a pore size of 200 μm (manufactured by PluriSelect, #43-50200-03), 50 mL of D-PBS(−) (manufactured by FUJIFILM Wako Pure Chemical Corporation, #045-29795) was added to wash the spheres trapped on the mesh. The mesh was turned upside down and washed with an appropriate amount of D-PBS (−) to collect the spheres trapped on the mesh. After centrifugation (300×g, 3 min, Decel mode), the supernatant was removed. Thereafter, 9723 μL of D-PBS(−) and 277 μL of an enzyme solution in which Liberase MNP-S 35 mg (manufactured by CustomBiotech, #05578566001) was dissolved in 14 mL of D-PBS(−) were added, and the mixture was incubated for 30 min in a 37° C. water bath, and pipetting was performed 20 times every 10 min. Thereafter, 10 mL of mesenchymal stem cell growth medium 2 was added, and the mixture was passed through a cell strainer (manufactured by Nissan Chemical Corporation) with a pore size of 65 μm using a syringe to obtain a filtrate containing single cells from which the substrates had been removed. After centrifugation (300×g, 3 min, Decel mode), the supernatant was removed, 10 mL of mesenchymal stem cell growth medium 2 was added, and the cell concentration was measured using a cell counter (manufactured by BIO-RAD, TC-20) to obtain 4.5×10⁵ cells.

### (Cell Staining)

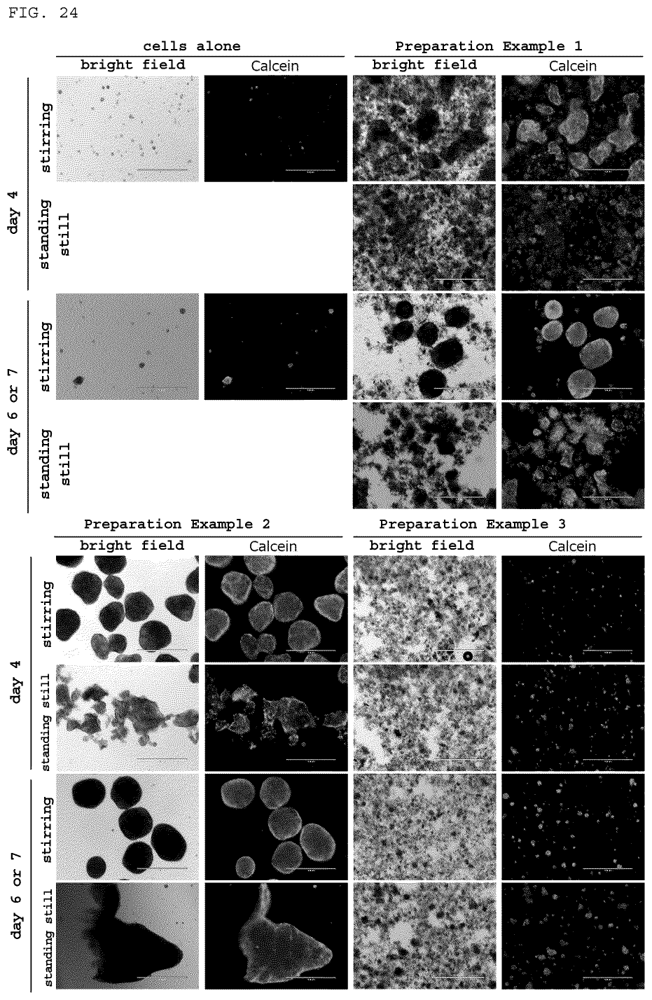

1 mL of culture medium seeded after uniform suspending (day 0 of culture) and culture medium on day 6 of culture were collected into 1.5 mL tubes, and after centrifugation (300×g, 3 min, Decel mode), the culture supernatant was removed. The cells were suspended in 1 mL of D-PBS(−) (manufactured by FUJIFILM Wako Pure Chemical Corporation, #045-29795), centrifuged (300×g, 3 min, Decel mode), and the culture supernatant was removed. A Calcein-AM (manufactured by DOJINDO LABORATORIES, #C326) solution (20 μL) dissolved in DMSO at a final concentration of 0.5 mg/mL was dissolved in 10 mL of D-PBS(−) (manufactured by FUJIFILM Wako Pure Chemical Corporation, #045-29795) and used as a staining solution. The cells were suspended in 1 mL of the staining solution, transferred to a 12-well plate (manufactured by Corning, #351143), and incubated in a CO₂ incubator (37° C., 5% CO₂) for 15 min. Thereafter, bright field images and viable cell-specific fluorescent staining images were obtained using EVOS (registered trade mark) FL Auto (manufactured by ThermoFisher). The results are shown in FIG. 23. The scale bar indicates 1000 μm.

As shown in FIG. 23, no spheres were observed in the cells subjected to operations 2 and 3 on day 0, but spheres were observed on day 6. In cells that underwent operation 1, spheres were observed even on day 0 because the operation only added new substrates to the spheres, but on day 6, sparse spheres with low fluorescence intensity appeared, and cells were observed to migrate from the already formed spheres to the newly added substrates. From the above results, it was confirmed that cells could be passaged efficiently in both substrate 1 and substrate 2, not only by a method of once dispersing spheres into single cells and then seeding the single cells into a fresh medium containing new substrates, but also by a method of adding a fresh medium containing new substrates to the formed spheres.

### Preparation Example 3

A 2% by mass chitosan nanofiber aqueous dispersion prepared according to the description in WO2015/111686 was sterilized in an autoclave at 121° C. for 20 min. Thereafter, this aqueous dispersion was mixed and suspended in sterile distilled water (Otsuka Distilled Water, manufactured by Otsuka Pharmaceutical Factory Co., Ltd.) to a concentration of 1% (w/v) to produce an aqueous dispersion containing sterile chitosan nanofibers. (In the present specification, the chitosan nanofiber prepared here is sometimes simply referred to as “substrate of Preparation Example 3”, “Preparation Example 3”, or “Substrate 3”).

### [Experimental Example 21] Comparison of Combination of Substrate and Stirring Conditions

Human adipose tissue-derived mesenchymal stem cells (manufactured by CellSource Co., Ltd., #0111201) were adhesion cultured for 3 days on a 15 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 30 mL of a medium composition in which the substrate of Preparation Example 1 was added to mesenchymal stem cell growth medium 2 to a final concentration 0.05% (w/v), the substrate of Preparation Example 2 to a final concentration 0.01% (w/v), or the substrate of Preparation Example 3 to a final concentration 0.04% (w/v), or 30 mL of a medium free of substrates, such that the seeding concentration was 1.5×10⁴ cells/mL, and cultured for 6 or 7 days under stirring conditions of 50 rpm in a CO₂ incubator (37° C., 5% CO₂). A 30 mL single-use reactor (manufactured by ABLE Corporation, #BWV-S03A) as a culture container, and an exclusive magnetic stirrer (manufactured by ABLE Corporation, #BWS-S03N0S-6) were used. In addition, the cells were added to 5 mL of a medium composition adjusted similarly. As a culture container, EZ-BindShut (registered trade mark) SP (low adhesive surface) 6-well plate (manufactured by AGC Techno Glass Co., Ltd., #4810-800SP) was used, and the cells were cultured under static conditions in a CO₂ incubator (37° C., 5% CO₂) for 7 days. On day 4 of culture, the culture container was allowed to stand for 10 min, and half of the culture supernatant was replaced with the medium.

### (Cell Staining)

1 mL of a uniformly suspended culture medium was collected into 1.5 mL tubes, and after centrifugation (300×g, 3 min, Decel mode), the culture supernatant was removed. The cells were suspended in 1 mL of D-PBS(−) (manufactured by FUJIFILM Wako Pure Chemical Corporation, #045-29795), centrifuged (300×g, 3 min, Decel mode), and the culture supernatant was removed. A Calcein-AM (manufactured by DOJINDO LABORATORIES, #C326) solution (20 μL) dissolved in DMSO at a final concentration of 0.5 mg/mL was dissolved in 10 mL of D-PBS(−) (manufactured by FUJIFILM Wako Pure Chemical Corporation, #045-29795) and used as a staining solution. The cells were suspended in 1 mL of the staining solution, transferred to a 12-well plate (manufactured by Corning, #351143), and incubated in a CO₂ incubator (37° C., 5% CO₂) for 15 min. Thereafter, bright field images and viable cell-specific fluorescent staining images were obtained using EVOS (registered trade mark) FL Auto (manufactured by ThermoFisher). The results are shown in FIG. 24. The scale bar indicates 1000 μm.

As shown in FIG. 24, when suspension culture was performed under stirring conditions using substrate 1 and substrate 2, spheres with clear outlines were obtained. On the other hand, when substrate 3 was used, smaller spheres were observed than when substrate 1 or substrate 2 was used. Further, when no substrate was used, almost no spheres were formed. Further, when substrate 2 was used under static conditions, large aggregates were formed, and the dispersibility of the spheres was lower than when substrate 1 was used under static conditions. From the above results, it was shown that culturing using the substrate of Preparation Example 1 or Preparation Example 2 under stirring conditions is effective in obtaining spheres having a uniform size.

### (Calculation of Proliferation Rate)

On days 0, 4, 6, or 7 of culture, 0.5 mL of uniformly suspended culture medium was collected, and 0.5 mL of ATP reagent (CellTiter-Glo (registered trade mark) Luminescent Cell Viability Assay, manufactured by Promega) was added to each. The mixture was stirred with a vortex, allowed to stand at room temperature for 10 min, and dispensed into a white 96-well plate by 100 μL. The luminescence intensity (RLU value) was measured by Enspire (manufactured by Perkin Elmer) and the number of viable cells was measured by subtracting the luminescence value of the medium alone. The relative value when the RLU value on day 0 of culture was set to 1 was defined as the cell proliferation rate. The results are shown in Table 23. The symbol “-” in the Table indicates not measured.

TABLE 23

| | stirring | | | | standing still | | |
|---|---|---|---|---|---|---|---|
| | cells | sub- | sub- | sub- | sub- | sub- | sub- |
| | alone | strate 1 | strate 2 | strate 3 | strate 1 | strate 2 | strate 3 |
| day | 4.1 | 14.1 | 23.2 | 6.3 | 16.1 | 10.3 | 7.6 |
| 4 | | | | | | | |
| day | 2.6 | — | — | — | — | — | — |
| 6 | | | | | | | |
| day | — | 27.8 | 31.5 | 12.9 | 22.7 | 18.9 | 15.1 |
| 7 | | | | | | | |

As shown in Table 23, the number of cells increased over time in all substrates, but substrate 1 and substrate 2 showed a higher proliferation rate on day 7 under stirring conditions than under static conditions. Furthermore, under stirring conditions, the growth rate was higher in substrate 2 than in substrate 1. On the other hand, cells did not proliferate when no substrate was used.

### [Experimental Example 22] Examination of Scale-Up

Human adipose tissue-derived mesenchymal stem cells (manufactured by CellSource Co., Ltd., #0111201) were adhesion cultured for 3 days on a 15 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210). After sterilizing a 1 L culture glass tank (manufactured by ABLE Corporation) with triangular blades attached thereto by autoclaving (121° C., 20 min), mesenchymal stem cell growth medium 2 and Penicillin-Streptomycin Solution (×100) (manufactured by FUJIFILM Wako Pure Chemical Corporation, #168-23191) were added, and placed in a BCP type animal cell culture device (manufactured by ABLE Corporation), and the culture medium was conditioned for 30 min under the conditions of 37° C. and 30 rpm under the control of 140 ccm of compressed air and appropriate addition of CO₂ to adjust the pH to 7.5. The detached cells were seeded at a seeding concentration of 1.5×10⁴ cells/mL in a medium in which the substrate of Preparation Example 1 was conditioned in advance to a final concentration of 0.05% (w/v) or the substrate of Preparation Example 2 was conditioned in advance to a final concentration of 0.01% (w/v). The total amount of medium was set to 1000 mL. Culture was performed under control conditions similar to those for conditioning. In addition, the detached cells were added at a seeding concentration of 1.5×10⁴ cells/mL to 100 mL of a medium composition in which the substrate of Preparation Example 1 was added to mesenchymal stem cell growth medium 2 to a final concentration of 0.05% (w/v) or the substrate of Preparation Example 2 was added to mesenchymal stem cell growth medium 2 to a final concentration of 0.01% (w/v), and cultured in a CO₂ incubator (37° C., 5% CO₂) under stirring condition of 50 rpm. A 100 mL single-use reactor (manufactured by ABLE Corporation, #BWV-S10A) as a culture container, and an exclusive magnetic stirrer (manufactured by ABLE Corporation, #BWS-S03N0S-6) were used. When the substrate of Preparation Example 1 was used, the cells were cultured for 7 days, and on day 4 of culture, the culture container was allowed to stand for 10 min, and half of the culture supernatant was replaced with the medium. When the substrate of Preparation Example 2 was used, the cells were cultured for 4 days.

### (Calculation of Proliferation Rate)

On days 0, 4, and 7 of culture when the substrate of Preparation Example 1 was used, and on days 0 and 4 of culture when the substrate of Preparation Example 2 was used, 0.5 mL of the uniformly suspended culture solution was collected, and 0.5 mL of ATP reagent (CellTiter-Glo (registered trade mark) Luminescent Cell Viability Assay, manufactured by Promega) was added to each. The mixture was stirred with a vortex, allowed to stand at room temperature for 10 min, and dispensed into a white 96-well plate by 100 μL. The luminescence intensity (RLU value) was measured by Enspire (manufactured by Perkin Elmer) and the number of viable cells was measured by subtracting the luminescence value of the medium alone. The relative value when the RLU value on day 0 of culture was set to 1 was defined as the cell proliferation rate. The results are shown in Table 24.

TABLE 24

| | culture medium | | proliferation rate | |
|---|---|---|---|---|
| conditions | amount | | day 4 | day 7 |
| Preparation | 100 | mL | 16.1 | 44.7 |
| Example 1 | 1 | L | 19.7 | 55.9 |
| Preparation | 100 | mL | 15.1 | — |
| Example 2 | 1 | L | 18.7 | — |

As shown in Table 24, a proliferation rate equivalent to or higher than that of 100 mL was obtained on a 1 L scale in all substrates. The above results suggest that scale-up is possible while maintaining the efficiency.

### (Cell Staining)

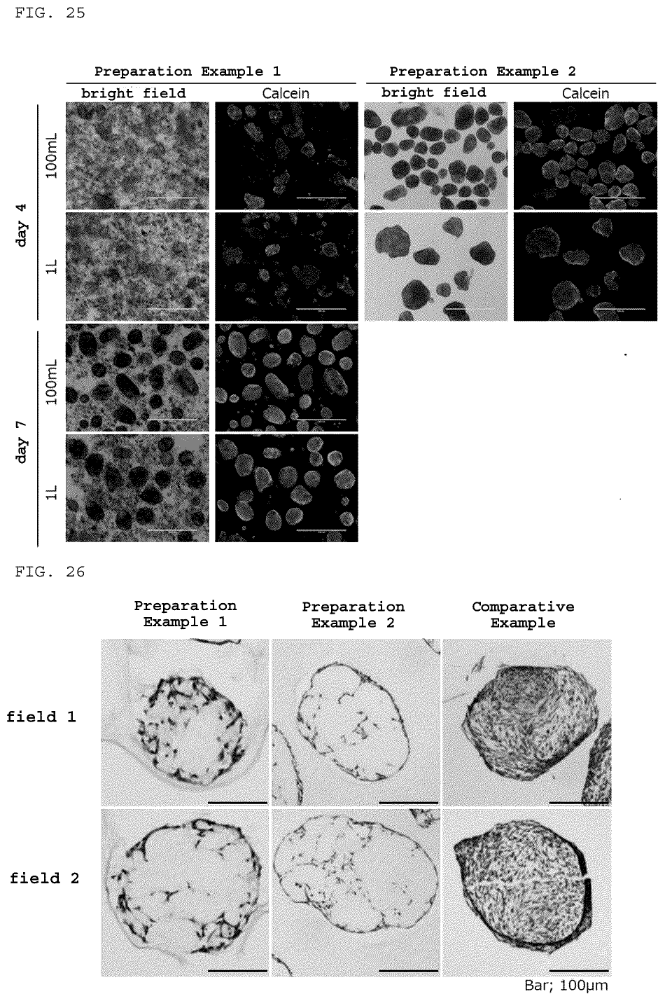

1 mL of a uniformly suspended culture medium was collected into 1.5 mL tubes, and after centrifugation (300×g, 3 min, Decel mode), the culture supernatant was removed. The cells were suspended in 1 mL of D-PBS(−) (manufactured by FUJIFILM Wako Pure Chemical Corporation, #045-29795), centrifuged (300×g, 3 min, Decel mode), and the culture supernatant was removed. A Calcein-AM (manufactured by DOJINDO LABORATORIES, #C326) solution (20 μL) dissolved in DMSO at a final concentration of 0.5 mg/mL was dissolved in 10 mL of D-PBS(−) (manufactured by FUJIFILM Wako Pure Chemical Corporation, #045-29795) and used as a staining solution. The cells were suspended in 1 mL of the staining solution, transferred to a 12-well plate (manufactured by Corning, #351143), and incubated in a CO₂ incubator (37° C., 5% CO₂) for 15 min. Thereafter, bright field images and viable cell-specific fluorescent staining images were obtained using EVOS (registered trade mark) FL Auto (manufactured by ThermoFisher). The results are shown in FIG. 25. The scale bar indicates 1000 μm.

As shown in FIG. 25, spheres equivalent to 100 mL on a 1 L scale were obtained for all substrates.

### [Experimental Example 23] Observation of Sphere Section

Human adipose tissue-derived mesenchymal stem cells (manufactured by CellSource Co., Ltd., #0111201) were adhesion cultured for 3 days on a 15 cm dish (manufactured by Corning, #430167) using mesenchymal stem cell growth medium 2 (manufactured by PromoCell, #C-28009). Thereafter, the cells were detached using DetachKit (manufactured by PromoCell, #C-41210), added to 30 mL or 100 mL of a medium composition in which the substrate of Preparation Example 1 was added to mesenchymal stem cell growth medium 2 to a final concentration 0.05% (w/v) or the substrate of Preparation Example 2 was added to mesenchymal stem cell growth medium 2 to a final concentration 0.01% (w/v), such that the seeding concentration was 1.5×10⁴ cells/mL, and cultured for 4 or 7 days under stirring conditions of 50 rpm in a CO₂ incubator (37° C., 5% CO₂). A 30 mL (manufactured by ABLE Corporation, #BWV-S03A) or 100 mL single-use reactor (manufactured by ABLE Corporation, #BWV-S10A) as a culture container, and an exclusive magnetic stirrer (manufactured by ABLE Corporation, #BWS-S03N0S-6) were used. As Comparative Example, the detached cells were added to mesenchymal stem cell growth medium 2, seeded on PrimeSurface (registered trade mark) plate 96U (manufactured by SUMITOMO BAKELITE CO., LTD., #MS-9096U) at 1.5×10⁴ cells/well/200 μL, and subjected to static culture in a CO₂ incubator (37° C., 5% CO₂) for 4 days. For the spheres obtained using the substrate of Preparation Example 1, on day 7 of culture, the culture medium was passed through a cell strainer with a pore size of 200 μm (manufactured by pluriSelect, #43-50200-03), washed with D-PBS(−), the mesh was turned upside down, and the spheres trapped on the mesh were collected using D-PBS(−) and used. The spheres obtained using the substrate of Preparation Example 2 and the spheres obtained under the conditions of Comparative Example used were those on day 4 of culture.

### (Preparation of Frozen Section)

After centrifuging the spheres (200×g, 3 min), the supernatant was removed, suspended in 50 μL of mesenchymal stem cell growth medium 2, and cooled on ice. The spheres were made into jelly according to the package insert of iPGell (manufactured by GENOSTAFF CO., LTD., #PG20-1). Specifically, L of ice-cold A-solution was added to the sphere suspension and sufficiently pipetted, and then 50 μL of B-solution at room temperature was added and immediately pipetted three times. After allowing the sample tube to stand at room temperature for 1 min, jelly-like solidification thereof was confirmed. Thereafter, 4% paraformaldehyde/phosphate buffer (manufactured by FUJIFILM Wako Pure Chemical Corporation, #163-20145) was added and immobilized by incubation overnight. Thereafter, the sample was washed with D-PBS(−), sucrose (manufactured by FUJIFILM Wako Pure Chemical Corporation, #196-00015) was dissolved in D-PBS(−) at a final concentration of 10 or 20 or 30% (w/v), and the sample was immersed in a 10% sucrose PBS solution for 4 hr, immersed in a 20% sucrose PBS solution overnight, and immersed in a 30% sucrose PBS solution overnight. Thereafter, the jelly-formed spheres were placed in a plastic embedding dish (manufactured by Sakura Finetech Japan Co., Ltd., #4730) containing frozen embedding compound (manufactured by Leica Microsystems, #3801480), and frozen in a 1:1 hexane/isopentane solution cooled to −100° C. using a desktop cooling trap (manufactured by TOKYO RIKAKIKAI CO., LTD., UT-2000). The prepared frozen embedded block was sliced into 10-30 μm slices using a cryostat (manufactured by Leica Microsystems, CM3050s) and attached to a slide glass (manufactured by Matsunami Glass Ind., Ltd., #S7445). The compound on the slide glass was removed with running water, immersed in hematoxylin (manufactured by Sakura Finetek Japan Co., Ltd., #6187-4P) for 5 min at room temperature, and then washed with running water for 5 min. Thereafter, it was dehydrated and cleared according to conventional methods, and encapsulated with a cover glass (manufactured by Matsunami Glass Ind., Ltd., #C024321) and an encapsulating agent (manufactured by FALMA, #308-600-1), and observed using an inverted microscope (manufactured by Olympus Corporation, #IX73). The obtained images are shown in FIG. 26. The scale bar indicates 100 μm.

As shown in FIG. 26, in Comparative Example, the cell nucleus was stained to the inside of the spheres, but in the spheres of substrate 1 and substrate 2, the inside of the sphere was not stained. From the above results, it was suggested that the spheres obtained using the substrate of Preparation Example 1 or 2 may embrace the substrate inside. In a sphere as in Comparative Example where cells are densely packed to the inside, there is a possibility that nutrients, oxygen, and the like from the outside cannot reach the center of the sphere, and the cells in the center of the sphere die. On the other hand, in the spheres prepared using the method of the present invention, the distance from the sphere surface to the center increases because the substrate is embraced inside the sphere, thus decreasing the number of cells to which nutrients and oxygen are difficult to reach. Therefore, according to the present invention, cells can be proliferated more efficiently.

### [Experimental Example 24] Application of Mesenchymal Stem Cell Prepared Using the Method of the Present Invention to Knee Osteoarthritis

### (Preparation of Adhesion Cultured Cells (2D Group))

Human adipose tissue-derived mesenchymal stem cells (manufactured by CellSource Co., Ltd., #0111201) were adhesion cultured for 3 days on a 15 cm dish (manufactured by Corning, #430167) at a seeding density of 5000 cells/cm² and using mesenchymal stem cell growth medium 2 (manufactured by PromoCell, #C-28009). Thereafter, the cells were detached using DetachKit (manufactured by PromoCell, #C-41210), and the cell concentration was counted using a cell counter (manufactured by BIO-RAD, #TC-20). The cells in a necessary number were separated into a new centrifuge tube, centrifuged (200×g, 3 min), and the supernatant was removed. The cells were suspended in STEMCELLBANKER GMP grade (manufactured by NIPPON ZENYAKU KOGYO CO., LTD., #CB045) at 5×10⁶ cells/vial, stored at −80° C. in CoolCell LX (manufactured by Corning, #432002), and stored in liquid nitrogen the next day.

### (Preparation of Stirring Cultured Cells (3D Group))

Human adipose tissue-derived mesenchymal stem cells (manufactured by CellSource Co., Ltd., #0111201) were adhesion cultured for 3 days on a 15 cm dish (manufactured by Corning, #430167) at a seeding density of 5000 cells/cm² and using mesenchymal stem cell growth medium 2 (manufactured by PromoCell, #C-28009). Thereafter, the cells were detached using DetachKit (manufactured by PromoCell, #C-41210), added to 100 mL of a medium composition in which the substrate of Preparation Example 1 was added to mesenchymal stem cell growth medium 2 to a final concentration 0.05% (w/v), such that the seeding concentration was 1.5×10⁴ cells/mL, and cultured for 7 days under stirring condition of 50 rpm in a CO₂ incubator (37° C., 5% CO₂). A 100 mL single-use reactor (manufactured by ABLE Corporation, #BWV-S10A) as a culture container and an exclusive magnetic stirrer (manufactured by ABLE Corporation, #BWS-S03N0S-6) were used. On day 4 of culture, the culture container was allowed to stand for 10 min, and half of the culture supernatant was replaced with the medium.

### (Separation of Spheres)

On day 7 of culture, the entire amount of the culture medium was passed through a cell strainer with a pore size of 200 μm (manufactured by pluriSelect, #43-50200-03), it was washed with 100 mL of D-PBS(−), the mesh was turned upside down to collect the spheres trapped on the mesh with 50 mL of the D-PBS(−). Thereafter, spheres were precipitated by natural sedimentation, and the supernatant was removed to give 10 mL of a cell suspension.

### (Enzyme Treatment)

554 μL of Liberase (registered trade mark) TM Research Grade (manufactured by Merck, #5401119001) solution dissolved in D-PBS(−) at a final concentration of 13 U/mL, 2 mL of TrypLE (registered trade mark) Select Enzyme (10×), no phenol red (manufactured by Thermo Fisher, #A1217701), and 7446 μL of D-PBS(−) were mixed to prepare 10 mL of an enzyme solution. The enzyme solution heated to 37° C. was added to a cell suspension and transferred to a cell dispersion tool (manufactured by ABLE Corporation). A cell dispersion tool was set in a high-rotation stirrer with a temperature control function for dispersion tool (manufactured by ABLE Corporation) heated to 37° C., and the cells were dispersed at 1200 rpm for 20 min.

### (Purification/Cryopreservation)

After pipetting 50 times using Eppendorf Research (registered trademark) plus100-1000 μL (manufactured by Eppendorf, #3120000062) with the discharge volume set to 1 mL, 20 mL of mesenchymal stem cell growth medium 2 was added to neutralize the enzyme. The cell suspension was filled into a 100 mL syringe, passed through a cell strainer (manufactured by Nissan Chemical Corporation) with a pore size of 65 μm, and the substrates were removed to obtain a filtrate containing single cells, and the cell concentration was counted using a cell counter (manufactured by BIO-RAD, #TC-20). The cells in a necessary number were separated into a new centrifuge tube, centrifuged (200×g, 3 min), and the supernatant was removed. The cells were suspended in STEMCELLBANKER GMP grade (manufactured by NIPPON ZENYAKU KOGYO CO., LTD., #CB045) at 5×10⁶ cells/vial, stored at −80° C. in CoolCell LX (manufactured by Corning, #432002), and stored in liquid nitrogen the next day.

### (Quantification of Remaining Amount of Substrates in 3D Group)

3×10⁵, 1×10⁶, or 1×10⁷ cells were separated from the cell suspension after purification, centrifuged (300×g, 3 min, Decel mode), the culture supernatant was removed, and the cells were lysed by adding and suspending in 1 mL of Reagent A100 (manufactured by Chemometec, #910-0003). Then, the remaining substrates contained in the cell suspension were quantified using Chitosan Assay Kit (manufactured by Cell Biolabs, #XAN-5126). After centrifuging 1 mL of the cell lysate (12300×g, 3 min), 0.9 mL of the supernatant was removed, 0.4 mL of 25M sodium hydroxide aqueous solution was added, and the mixture was heated at 121° C. for 3 hr, whereby a deacetylation treatment was performed. Next, 1 mL of ethanol was added and vortex stirred (2500 speed, 1 min) to precipitate the polymer. After repeating centrifugation (12300×g, 3 min) and supernatant removal (1000 μL) twice, sodium hydroxide was removed by further adding 1 mL of water (12,300×g, 3 min) and the supernatant was removed (1300 μL), and the sample was dried under reduced pressure. The dried sample was dissolved by adding 0.2 mL of acetate buffer for measurement, and the sample was subjected to pre-measurement treatment according to the aforementioned kit procedural manual. The sample was dispensed by 250 μL per well into wells of a 96-well plate with a transparent bottom and white side (manufactured by Corning, #3632) and the absorbance at 540 nm was measured using a plate reader (manufactured by Tecan, infinite M200PRO). The concentration of the substrate contained in the sample was calculated using a calibration curve drawn using the standard material included in the aforementioned kit. The number of cells and the amount of the substrate contained in the sample are shown in Table 25. In addition, a calibration curve formula was derived from Table 25, and used to estimate the amount of substrate contained in the 0.975×10⁴ and 7.5×10⁵ cell suspensions (Table 26).

TABLE 25

| number | liquid | measurement | amount of substrate |
|---|---|---|---|
| of cells | amount | value | contained |
| (cells) | (mL) | (ug/mL) | (ug) |
| 3 × 10\hat{ }5 | 0.2 | 0.56 | 2.78 |
| 1 × 10\hat{ }6 | 0.2 | 0.99 | 4.95 |
| 1 × 10\hat{ }7 | 0.2 | 1.63 | 8.14 |

### (Calibration Curve Formula)

y: substrate amount (μg)
x: number of cells (×10⁵ cells)
y=0.0469x+3.5253

TABLE 26

| | assumed amount of |
|---|---|
| number of cells | substrate contained |
| (cells) | (ug) |
| 7.5 × 110\hat{ }5 | 3.88 |

### (Obtaining Sample for Measurement of Amount of PGE2 Produced)

In 2D group, the cells were seeded in 1 mL of mesenchymal stem cell growth medium 2 at a seeding density of 4000 cells/cm² in a 24-well plate (manufactured by Corning, #3526), and after 4 days of culture, the supernatant was removed, washed with D-PBS(−), 1 mL of mesenchymal stem cell growth medium 2 or mesenchymal stem cell growth medium 2 containing TNF-α (manufactured by R&D Systems, #210-TA) at a final concentration of 20 ng/mL was added, and cultured in a CO₂ incubator (37° C., 5% CO₂), for 24 hr. The culture supernatant was used as a PGE2 measurement sample, and the cells were used as an ATP measurement sample. In 3D group, 1.2 mL of the uniformly suspended culture medium on day 7 of culture was collected into a 1.5 mL tube, and after centrifugation (300×g, 3 min, Decel mode), the culture supernatant was removed. Thereafter, the cells were lysed by adding and suspending in 1.2 mL of Reagent A100 (manufactured by Chemometec, #910-0003), and 100 μL was collected into a 1.5 mL tube. 100 μL of Reagent B (manufactured by Chemometec, #910-0002) was added, loaded into Vial-Cassette (registered trademark) (manufactured by Chemometec, #941-0012), and the cell concentration was counted using NucleoCounter (registered trademark) NC-200 (registered trademark) (manufactured by Chemometec). 1×10⁵ cells were collected into a centrifuge tube, centrifuged (300×g, 3 min, Decel mode), the culture supernatant was removed, washed with D-PBS(−), 1 mL of mesenchymal stem cell growth medium 2 or mesenchymal stem cell growth medium 2 containing TNF-α (manufactured by R&D Systems, #210-TA) at a final concentration of 20 ng/mL was added, and cultured in a CO₂ incubator (37° C., 5% CO₂) on a 24-well ultra-low adhesive surface plate (manufactured by Corning, #3473) in a static state for 24 hr. After culturing, the supernatant was obtained by centrifugation (300×g, 3 min, Decel mode) and used as a PGE2 measurement sample, and the cells were used as an ATP measurement sample.

### (Calculation of Number of Cells and Measurement of Amount of PGE2 Produced)

1 mL of ATP reagent (CellTiter-Glo (registered trade mark) Luminescent Cell Viability Assay, manufactured by Promega) was added and the cells were suspended by pipetting. The mixture was allowed to stand at room temperature for 10 min, and dispensed into a white 96-well plate by 150 μL. The luminescence intensity (RLU value) was measured by Enspire (manufactured by Perkin Elmer) and the ATP value was measured by subtracting the luminescence value of the medium alone and used as the number of viable cells. Then, PGE2 contained in the recovered culture supernatant was quantified using PGE2 ELISA kit (manufactured by EnzoLife Science, #ADI-900-001). The standard diluted with Assay Buffer and the culture supernatant (100 μL) were added to each well of the 96-well plate included in the kit. Successively, 50 μL of blue conjugate was added to each well. Furthermore, 50 μL of yellow antibody was added to each well, and the mixture was shaken under room temperature conditions for 2 hr. Successively, the solution was discarded, a wash solution was added at 400 μL/well, and the solution was discarded. The above-mentioned operation was repeated 3 times. 200 μL of pNpp substrate solution was added to each well, and the mixture was shaken under room temperature conditions for 45 min. Finally, 50 μL of stop solution was added to quench the reaction, and the absorbance at 405 nm was measured. The concentration of PGE2 contained in each sample was calculated from 4-parameter logistic regression of the calibration curve. To calculate the amount of secretion per unit number of cells, a relative value was calculated by dividing the calculated amount of PGE2 by the ATP value. The results are shown in Table 27.

TABLE 27

| | TNF- | ATP (RLU) | | | PGE2 (pg/mL) | | | (PGE2/ATP) |
|---|---|---|---|---|---|---|---|---|
| | α | N = 1 | N = 2 | average | N = 1 | N = 2 | average | ×10\hat{ }6 |
| 2D | − | 7919310 | 7674405 | 7796858 | 38.19 | 43.14 | 40.67 | 5.2 |
| | + | 7008105 | 6937905 | 6973005 | 74.01 | 74.72 | 74.37 | 10.7 |
| 3D | − | 1451600 | 1092860 | 1272230 | 71.21 | 61.19 | 66.2 | 52 |
| | + | 1046890 | 825425 | 936158 | 333.4 | 192.5 | 262.95 | 280.9 |

As shown in Table 27, it was confirmed that the secreted amount of PGE2 increased in the 3D group as compared with the 2D group. The above results suggest that mesenchymal stem cells prepared using the method of the present invention may have higher anti-inflammatory action than the mesenchymal stem cells prepared by conventional adhesion culture.

(Analysis of cell surface markers) After washing cells in a single-cell state in 2D and 3D groups with washing buffer (D-PBS(−) containing 2% FBS), as specific staining antibodies, BV421 Mouse Anti-Human CD73 (manufactured by BD, #562430), APC Mouse Anti-Human CD90 (manufactured by BD, #559869), BV650 Mouse Anti-Human CD105 (manufactured by BD, #563466), FITC Anti-CD11b antibody [M1/70](manufactured by Abcam, #ab24874), and PE Mouse Anti-Human CD34 (manufactured by BD, #555822) were respectively added, and the cells were incubated on ice for 30 min in the dark. As a negative control, BV421 Mouse IgG1,k Isotype Control (manufactured by BD, #562438), APC Mouse IgG1,kappa Isotype Control (manufactured by BD, #555751), BV650 Mouse IgG1,k Isotype Control (manufactured by BD, #563231), FITC Rat IgG2b,kappa monoclonal[eB149/10H5]-Isotype control (manufactured by Abcam, #ab136125), and PE Mouse IgG1,kappa Isotype Control (manufactured by BD, #555749) were respectively added as control antibodies. After incubation, the cells were washed twice with washing buffer, treated with a 35 μm cell strainer, and then measured using BD LSRFortessa (registered trademark) X-20 (manufactured by BD), and the positive rate of each cell surface marker was calculated. CD73, CD90, and CD105 are positive markers for mesenchymal stem cells, and CD11b and CD34 are negative markers for mesenchymal stem cells. The results are shown in Table 28.

TABLE 28

| | | 2D group | | 3D group | |
|---|---|---|---|---|---|
| positive rate | | control | staining | control | staining |
| (%) | | antibody | antibody | antibody | antibody |
| positive | CD73 | 0.53 | 99.95 | 0.2 | 99.99 |
| marker | CD90 | 0.37 | 99.95 | 0.1 | 99.99 |
| | CD105 | 0.17 | 99.91 | 1.22 | 83.42 |
| negative | CD11b | 0.15 | 0.11 | 0.18 | 0.19 |
| marker | CD34 | 0.46 | 0.19 | 0.31 | 0.27 |

As shown in Table 28, the cells in the 2D group and 3D group both expressed CD73, CD90, and CD105, but did not express the negative markers CD11b and CD34. In other words, it was clarified that all of these cells maintained the state of mesenchymal stem cells.

(Production of Rat with Knee Osteoarthritis)

7-Week-old male Wistar rats (Japan SLC) were used. The target rats were anesthetized by subcutaneously administering a three-part mixed anesthetic solution (1.8 mL/kg). The final doses of each anesthetic were 2 mg/kg midazolam, 0.4 mg/kg medetomidine hydrochloride, and 5 mg/kg butorphanol tartrate. Simultaneously with anesthesia, an analgesic (carprofen 5 mg/kg, 1 mL/kg) was administered subcutaneously to treat the pain after operation. Then, the right hindpaw of the rat under systemic anesthesia was shaved, and the skin on the medial side of the patella was longitudinally dissected. Thereafter, the muscle tissue was dissected and the medial collateral ligament was exposed. The medial collateral ligament and anterior cruciate ligament were cut with MANI (registered trademark) Ophthalmic Knife (manufactured by MANI, INC., straight 22.5°), the meniscus was separated from the femur and tibia, and the meniscus was removed. After suturing with a suture thread or surgical stapler, iodine tincture was dripped onto the sutured area for disinfection. A mixture of two antagonists (1 mL/kg) was administered subcutaneously to wake up the animals from anesthesia. The final doses of the antagonists are atipamezole hydrochloride 1.2 mg/kg and flumazenil 0.01 mg/kg. After the animals became conscious, the presence of abnormalities in their general condition was checked. For rats in the sham surgery group, the knee skin was incised, then sutured and disinfected.

(Administration of Mesenchymal Stem Cells Prepared Using the Method of the Present Invention to Rats with Knee Osteoarthritis)

The rats were divided into groups as shown in the following Table 29. Three days after the surgery, the rats were anesthetized with 1.5-3.0% isoflurane, and then mesenchymal stem cells or a hyaluronic acid preparation (Svenyl Dispo Joint Injection 25 mg, manufactured by Chugai Pharmaceutical Co., Ltd., positive control) was injected into the right hind knee joint of each rat. Mesenchymal stem cells were administered once 3 days after surgery, and hyaluronic acid preparation was administered 4 times in total by 50 μL each on days 3, 10, 17, and 24 after surgery. The concentration of mesenchymal stem cells was adjusted with physiological saline (OTSUKA NORMAL SALINE, manufactured by Otsuka Pharmaceutical Factory Co., Ltd.), and a 1 mL syringe with a 26G needle was used for administration.

TABLE 29

| | | | concen- | | |
|---|---|---|---|---|---|
| | | | tration | | |
| | | | of | | |
| | | adminis- | adminis- | | |
| | | tration | tered | adminis- | |
| | dose | dose | solution | tration | number |
| group | (cells/knee) | (μL/knee) | (cells/mL) | frequency | of rats |
| sham surgery | 0 | 50 | 0 | 1 | 6 |
| group | | | | | |
| Control group | 0 | 50 | 0 | 1 | 10 |
| 2D group | 7.5 × 10⁵ | 50 | 1.5 × 10⁷ | 1 | 10 |
| 3D group | 7.5 × 10⁵ | 50 | 1.5 × 10⁷ | 1 | 10 |
| | | | concen- | | |
| | | | tration | | |
| | | | of | | |
| | | adminis- | adminis- | | |
| | | tration | tered | adminis- | |
| | dose | dose | solution | tration | number |
| group | (μg/knee) | (μL/knee) | (mg/mL) | frequency | of rats |
| SUVENYL | 0.5 | 50 | 10 | 4 | 10 |
| group | | | | | |

### (Bilateral Plantar Pressure Difference Pain Sensation Evaluation)

On day 27 after the surgery, Bilateral Plantar Pressure Difference Pain Sensation evaluation was performed. In order to obtain more accurate results, the individual numbers of the animals being evaluated were not disclosed to the evaluators, and the evaluation was conducted in a blinded manner. All rats were placed in a holder for Bilateral Plantar Pressure Difference Pain Sensation evaluation, and after being allowed to settle down for several minutes, the weight on the left and right hindlimbs was measured using a pressure difference pain measurement device (Bio Research Center). Measurements were performed until data could be obtained five times per rat. Weighting balance (R/L weighting) was calculated, and the average value of the weighting balance for each rat was determined. In addition, a significant difference test for the Control group was performed using the Tukey test, and the p value was calculated. The results are shown in Table 30.

TABLE 30

| | sham | | | | |
|---|---|---|---|---|---|
| | surgery | | | | |
| | group | Control | 2D group | 3D group | SUVENYL |
| average | 0.99 | 0.69 | 0.85 | 0.91 | 0.92 |
| SD | 0.0279 | 0.1104 | 0.0774 | 0.1074 | 0.088 |
| p value | — | — | 0.0032 | <0.001 | <0.001 |

As shown in Table 30, the weighting balance average values significantly increased in all cell administration groups compared to the Control group. This indicates that cell administration suppressed pain in rats. Furthermore, the average value of weighting balance indicates that the 3D group may have a higher pain suppressing effect than the 2D group.

### INDUSTRIAL APPLICABILITY

According to the present invention, adherent cells with good quality can be efficiently produced in a large amount. Therefore, the present invention is preferably used, for example, in preparing cells for transplantation into a living body. Therefore, the present invention can be extremely useful in the technical field of biological transplantation.

This application is based on a patent application No. 2021-169860 filed in Japan (filing date: Oct. 15, 2021) and a patent application No. 2022-023397 filed in Japan (filing date: Feb. 18, 2022), the contents of which are incorporated in full herein.

1. A method for culturing an adherent cell, comprising a step of suspension culturing the adherent cell in a medium comprising a nanofiber composed of a water-insoluble polysaccharide, wherein the culture is performed along with stirring.

2. The method according to claim 1, wherein the aforementioned stirring is performed under conditions where the nanofiber and the cell are suspended in the medium and the nanofiber and the cell are continuously moved in the system by an external force.

3. The method according to claim 1, wherein the aforementioned stirring is performed by a means accompanying a blade, and a rotation speed thereof is a tip speed of 0.01 to 50.0 m/min.

4. The method according to claim 1, wherein the aforementioned stirring is performed constantly during the cell culture.

5. The method according to claim 1, wherein a content of the nanofiber composed of water-insoluble polysaccharides and added to the medium is 0.0001-0.2% (w/v).

6. The method according to claim 1, wherein the nanofiber composed of water-insoluble polysaccharides carries an extracellular matrix.

7. The method according to claim 1, wherein the water-insoluble polysaccharide is at least one selected from the group consisting of chitin, cellulose, and hemicellulose.

8. The method according to claim 6, wherein the extracellular matrix is at least one selected from the group consisting of collagen, fibronectin, vitronectin, laminin, RGD sequence, and cadherin.

9. The method according to claim 1, wherein the adherent cell is selected from the group consisting of a stem cell, a progenitor cell, a somatic non-stem cell, a primary cultured cell, a cell line, and a cancer cell.

10. The method according to claim 1, wherein the medium further comprises a chitosan nanofiber.

11. A method for producing a sphere of adherent cells with a uniform sphere size, comprising a step of suspension culturing adherent cells in a medium comprising a nanofiber composed of water-insoluble polysaccharides, wherein the culture is performed along with stirring.

12. The method according to claim 11, wherein the aforementioned stirring is performed under conditions where the nanofibers and the cells are suspended in the medium and the nanofibers and the cells are continuously moved in the system by an external force.

13. The method according to claim 11, wherein the aforementioned stirring is performed by a means accompanying a blade, and a rotation speed thereof is a tip speed of 0.01 to 50.0 m/min.

14. The method according to claim 11, wherein the aforementioned stirring is performed constantly during the cell culture.

15. The method according to claim 11, wherein a content of the nanofiber composed of water-insoluble polysaccharides and added to the medium is 0.0001-0.2% (w/v).

16. A method for isolating spheres, comprising a step of subjecting a suspension of spheres produced by the method according to claim 11 to a cell strainer.

17. A method for dispersing adherent cells in the form of spheres into single cells, comprising
a first step of suspension culturing adherent cells in a medium comprising nanofibers composed of water-insoluble polysaccharides, and
a second step of treating the spheres of the adherent cells obtained in the first step with a cell dispersing agent.

18. A mesenchymal stem cell in which expression of at least one gene selected from the group consisting of CD55, HMOX1, TSPAN7, RAB27B, IL33, GPX3, and MFAP4 is promoted as compared with that in a mesenchymal stem cell cultured by adhesion culture.

19. The mesenchymal stem cell according to claim 18, wherein production of an extracellular vesicle is enhanced as compared with that in a mesenchymal stem cell cultured by adhesion culture.

20. The mesenchymal stem cell according to claim 19, wherein the extracellular vesicle is an exosome.

21. A method for enhancing production of an extracellular vesicle of a mesenchymal stem cell, comprising a step of suspension culturing the mesenchymal stem cell in a medium comprising a nanofiber composed of water-insoluble polysaccharides, wherein the culture is performed along with stirring.

22. A method for producing a mesenchymal stem cell in which production of an extracellular vesicle is enhanced, comprising a step of suspension culturing the mesenchymal stem cell in a medium comprising a nanofiber composed of water-insoluble polysaccharides, wherein the culture is performed along with stirring.

23. The method according to claim 21, wherein the extracellular vesicle is an exosome.

24. An agent for treating an inflammatory disease, comprising the mesenchymal stem cell according to claim 18.
